SEQUENCE LISTING (19 sequences)

SEQ ID NO: 1 - RNA, 21 nt, Artificial Sequence
  HBV RNAi Agent antisense strand modified sequence
agaaaauugagagaaguccac

SEQ ID NO: 2 - RNA, 21 nt, Artificial Sequence
  HBV RNAi Agent antisense strand modified sequence
agaaaauugagagaaguccac

SEQ ID NO: 3 - RNA, 23 nt, Artificial Sequence
  HBV RNAi Agent antisense strand modified sequence
agaaaauugagagaaguccacuu

SEQ ID NO: 4 - RNA, 22 nt, Artificial Sequence
  HBV RNAi Agent antisense strand modified sequence
agaaaauugagagaaguccacc

SEQ ID NO: 5 - RNA, 21 nt, Artificial Sequence
  HBV RNAi Agent antisense strand unmodified sequence
agaaaauugagagaaguccac

SEQ ID NO: 6 - RNA, 23 nt, Artificial Sequence
  HBV RNAi Agent antisense strand unmodified sequence
agaaaauugagagaaguccacuu

SEQ ID NO: 7 - RNA, 22 nt, Artificial Sequence
  HBV RNAi Agent antisense strand unmodified sequence
agaaaauugagagaaguccacc

SEQ ID NO: 8 - RNA, 21 nt, Artificial Sequence
  HBV RNAi Agent antisense strand modified sequence
uaccaauuuaugccuacagcg

SEQ ID NO: 9 - RNA, 21 nt, Artificial Sequence
  HBV RNAi Agent antisense strand unmodified sequence
uaccaauuuaugccuacagcg

SEQ ID NO: 10 - RNA, 21 nt, Artificial Sequence
  HBV RNAi Agent sense strand modified sequence
guggacuucucucaauuuucu

SEQ ID NO: 11 - RNA, 21 nt, Artificial Sequence
  HBV RNAi Agent sense strand modified sequence
guggacuucucucaauuuucu

SEQ ID NO: 12 - RNA, 22 nt, Artificial Sequence
  HBV RNAi Agent sense strand modified sequence
gguggacuucucucaauuuucu

SEQ ID NO: 13 - RNA, 21 nt, Artificial Sequence
  HBV RNAi Agent sense strand modified sequence
guggacuucucucaauuuucu

SEQ ID NO: 14 - RNA, 21 nt, Artificial Sequence
  HBV RNAi Agent sense strand unmodified sequence
guggacuucucucaauuuucu

SEQ ID NO: 15 - RNA, 22 nt, Artificial Sequence
  HBV RNAi Agent sense strand unmodified sequence
gguggacuucucucaauuuucu

SEQ ID NO: 16 - RNA, 21 nt, Artificial Sequence
  HBV RNAi Agent sense strand modified sequence
cgcuguaggcauaaauuggua

SEQ ID NO: 17 - RNA, 21 nt, Artificial Sequence
  HBV RNAi Agent sense strand modified sequence
cgcuguaggcauaaauuggua

SEQ ID NO: 18 - RNA, 21 nt, Artificial Sequence
  HBV RNAi Agent sense strand modified sequence
cgcuguaggcauaaauuggua

SEQ ID NO: 19 - RNA, 21 nt, Artificial Sequence
  HBV RNAi Agent sense strand unmodified sequence
cgcuguaggcauaaauuggua

(19) United States
(12) Patent Application Publication
(10) Pub. No.: US 2020/0332297 A1
(43) Pub. Date: Oct. 22, 2020

(54) COMBINATION THERAPY FOR TREATING HEPATITIS B VIRUS INFECTION

(71) Applicant: Janssen Pharmaceuticals, Inc., Titusville, NJ (US)

(72) Inventors: Michael BIERMER, Berlin (DE); Ronald KALMEIJER, Titusville, NJ (US); Oliver LENZ, Sint-Katelijne-Waver (BE); Maria Gloria BEUMONT-MAUVIEL, Bievres (FR); Jan SNOEYS, Westmalle (BE); Kenneth Alan SIMMEN, Oxfordshire (GB)

(73) Assignee: Janssen Pharmaceuticals, Inc., Titusville, NJ (US)

(21) Appl. No.: 16/851,679

(22) Filed: Apr. 17, 2020

Related U.S. Application Data
(60) Provisional application No. 62/932,332, filed on Nov. 7, 2019.
(60) Provisional application No. 62/852,743, filed on May 24, 2019.
(60) Provisional application No. 62/836,069, filed on Apr. 18, 2019.

(30) Foreign Application Priority Data
Aug. 9, 2019 (US) PCT/US2019/046035

(51) Int. Cl. C12N 15/113; A61K 31/40; A61K 31/522; A61K 31/675; A61K 47/54; A61P 31/20

(52) U.S. Cl. CPC C12N 15/1131; A61K 31/40; A61K 31/522; A61K 9/0019; A61K 47/555; A61P 31/20; A61K 31/675

(57) ABSTRACT

Described are RNA interference (RNAi) agents for inhibiting the expression of Hepatitis B Virus (HBV) used in combination with a capsid assembly modulator (CAM) and methods of administering same. The HBV RNAi agents and CAMs are administered in ratios to effectively inhibit HBV gene expression and to treat diseases and conditions associated with HBV infection.

### CROSS REFERENCE TO RELATED APPLICATIONS

This application claims the priority benefit of U.S. Provisional Patent Application Ser. No. 62/836,069, filed Apr. 18, 2019; U.S. Provisional Patent Application Ser. No. 62/852,743, filed May 24, 2019; PCT Patent Application No. PCT/US2019/046035, filed Aug. 9, 2019; and U.S. Provisional Patent Application Ser. No. 62/932,332, filed Nov. 7, 2019; the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165002000600SEQLIST.TXT, date recorded: Apr. 17, 2020, size: 8 KB).

### FIELD OF INVENTION

The present disclosure relates generally to compositions and kits comprising an RNA interference (RNAi) component and a capsid assembly modulator and their uses for treating Hepatitis B Virus infection or inhibiting the expression of at least one Hepatitis B Virus gene.

### BACKGROUND

The Hepatitis B Virus (HBV) is a strict hepatotrophic, double-stranded DNA containing virus. Although DNA is the genetic material, the replication cycle involves a reverse transcription step to copy a pregenomic RNA into DNA. Hepatitis B Virus is classified as one member of the Hepadnaviruses and belongs to the family of Hepadnaviridae. The primary infection of adult humans with Hepatitis B Virus causes an acute hepatitis with symptoms of organ inflammation, fever, jaundice and increased liver transaminases in blood. Those patients that are not able to overcome the virus infection suffer a chronic disease progression over many years with increased risk of developing cirrhotic liver or liver cancer. Perinatal transmission from Hepatitis B Virus-infected mothers to newborns also leads to chronic hepatitis.

Upon uptake by hepatocytes, the nucleocapsid is transferred to the nucleus and DNA is released. There, the DNA strand synthesis is completed and gaps repaired to give the covalently closed circular (ccc) supercoiled DNA of 3.2 kb. The cccDNA serves as a template for transcription of five major viral mRNAs, which are 3.5, 3.5, 2.4, 2.1 and 0.7 kb long. All mRNAs are 5′-capped and polyadenylated at the 3′-end. There is sequence overlap at the 3′-end between all five mRNAs.

One 3.5 kb mRNA serves as template for core protein and polymerase production. In addition, the same transcript serves as a pre-genomic replication intermediate and allows the viral polymerase to initiate the reverse transcription into DNA. Core protein is needed for nucleocapsid formation. The other 3.5 kb mRNA encodes pre-core, the secretable e-antigen (HBeAg). In the absence of replication inhibitors, the abundance of e-antigen in blood correlates with Hepatitis B Virus replication in liver and serves as an important diagnostic marker for monitoring the disease progression.

The 2.4 and 2.1 kb mRNAs carry the open reading frames (“ORF”) pre-S1, pre-S2 and S for expression of viral large, medium and small surface antigen. The s-antigen is associated with infectious, complete particles. In addition, blood of infected patients also contain non-infectious particles derived from s-antigen alone, free of genomic DNA or polymerase. The function of these particles is not fully understood. The complete and lasting depletion of detectable s-antigen in blood is considered as a reliable indicator for Hepatitis B Virus clearance.

The 0.7 kb mRNA encodes the X protein. This gene product is important for efficient transcription of viral genes and also acts as a transactivator on host gene expression. The latter activity seems to be important for hepatocyte transformation during development of liver cancer.

Patients with detectable s-antigen, e-antigen, and/or viral DNA in the blood for more than 6 months are considered chronically infected. Nucleoside analogs as inhibitors of reverse transcriptase activity are typically the first treatment option for many patients. Administration of lamivudine, tenofovir, and/or entecavir has been shown to suppress Hepatitis B Virus replication, sometimes to undetectable levels, with improvement of liver function and reduction of liver inflammation typically seen as the most important benefits. However, only few patients achieve complete and lasting remission after the end of treatment. Furthermore, the Hepatitis B Virus develops drug resistance with increasing duration of treatment. This is especially difficult for patients co-infected with Hepatitis B and Human Immunodeficiency Virus (HIV). Both viruses are susceptible to nucleoside analogue drugs and may co-develop resistance.

A second treatment option is the administration of interferon-alpha. Here, patients receive high doses of interferon-alpha over a period of 6 months. The Asian genotype B gives very poor response rates. Co-infection with Hepatitis D Virus (HDV) or Human Immunodeficiency Virus has been shown to render interferon-alpha therapy completely ineffective. Patients with strong liver damage and heavy fibrotic conditions are not qualified for interferon-alpha therapy.

Certain Hepatitis B Virus-specific RNA interference (RNAi) agents have been previously shown to inhibit expression of HBV gene expression. For example, U.S. Patent Application Publication No. 2013/0005793, to Chin et al., which is incorporated herein by reference in its entirety, discloses certain double-stranded ribonucleic acid (dsRNA) molecules for inhibiting the expression of Hepatitis B Virus gene.

Additionally, HBV inhibitors, such as capsid assembly modulator (CAM), can bind to Hepatitis B core protein and interferes with the viral capsid assembly process, thereby preventing the polymerase-bound pgRNA encapsidation. This results in the formation of HBV capsids, devoid of HBV DNA or RNA (non-functional capsids), and ultimately in the inhibition of HBV replication. See e.g., WO2014184350A1. The reference WO2014184350A1 is incorporated herein in its entirety, particularly the descriptions of the capsid assembly modulator compounds and the method of preparing them.

There is a need for improved HBV therapy that can overcome at least one of the disadvantages of existing treatment options, such as are toxicity, mutagenicity, lack of selectivity, poor efficacy, poor bioavailability, and difficulty of synthesis, while providing additional benefits such as increased potency or an increased safety window.

The disclosures of all publications, patents, patent applications and published patent applications referred to herein are hereby incorporated herein by reference in their entirety.

### BRIEF SUMMARY

Provided herein is A method for inhibiting the expression of a Hepatitis B Virus gene in a subject in need thereof, wherein the method comprises administering to the subject an effective amount of an RNAi component and a compound of Formula (I), wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
   (b) the compound of Formula (I) is

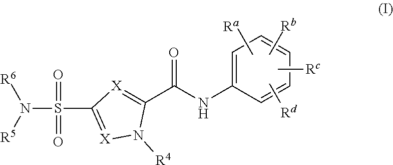

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is a method for treating a disease or disorder associated with an infection caused by Hepatitis B Virus in a subject, wherein the method comprises administering to the subject an effective amount of an RNAi component and a compound of Formula (I), wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

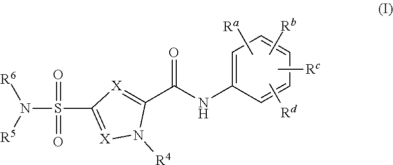

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is a method of treating a disease or disorder associated with an infection caused by Hepatitis B Virus in a subject receiving a capsid assembly modulator therapy, wherein the method comprises administering to the subject an effective amount of an RNAi component, wherein the RNAi component comprises
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
(ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19.

Also provided herein is a method for inhibiting the expression of a Hepatitis B Virus gene in a subject in need thereof, wherein the subject is administered an effective amount of the compound of Formula (I) in combination with an RNAi component, wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

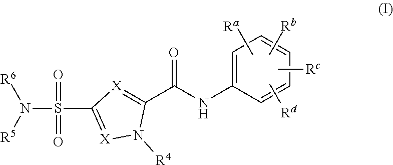

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is a method of treating a Hepatitis B Virus infection comprising contacting a cell infected with the Hepatitis B Virus infection with an effective amount of an RNAi component and a compound of Formula (I), or a therapeutically effective metabolite of the foregoing, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

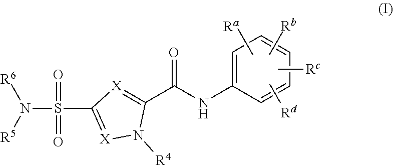

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is a kit comprising an effective amount of an RNAi component and a compound of Formula (I) or a pharmaceutically acceptable salt thereof, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

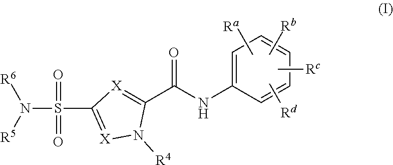

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is a composition comprising an RNAi component and a compound of Formula (I), wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

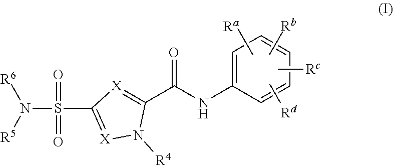

or a pharmaceutically acceptable salt thereof, wherein:
each X independently is CR⁷;
R^{a}, R^{b} and R^{c} are independently selected from the group consisting of hydrogen, fluoro, bromo, chloro, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally being substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, fluoro, chloro, bromo, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

Also provided herein is an effective amount of an RNAi component and a compound of Formula (I) in the manufacture of a medicament for treating a viral infection in a subject caused by Hepatitis B Virus, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

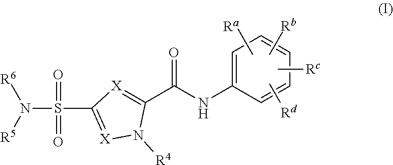

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### BRIEF DESCRIPTION OF THE DRAWINGS

FIG. 1 shows the individual changes in HBsAg from Day 1 to Day 29 for CHB patients (cohort 12 of Example 1) receiving (1) 200 mg RNAi component, (2) 250 mg compound A daily and (3) NA. The patients were given a single subcutaneous dose of the RNAi component on Day 1. Each line represents one individual CHB patient.

FIG. 2 shows the mean log HBsAg change from Day 1 to Day 29 for CHB patients (cohort 12 of Example 1) receiving (1) 200 mg RNAi component, (2) 250 mg compound A daily and (3) NA. The patients were given a single subcutaneous dose of the RNAi component on Day 1.

FIGS. 3A-3C show the individual changes in HBsAg from Day 1 to Day 85 (FIG. 3A), Day 1 to Day 168 (FIG. 3B), or Day 1 to Day 336 (FIG. 3C) for CHB patients (cohort 12 of Example 1) receiving (1) 200 mg RNAi component, (2) 250 mg compound A daily and (3) NA. The patients were given three subcutaneous doses of the RNAi component on Day 1, 29 and 57. Each line represents one individual CHB patient. Dotted lines represent HBeAg positive patients.

FIG. 4 shows the mean log HBsAg change from Day 1 to Day 85 for CHB patients (cohort 12 of Example 1) receiving (1) 200 mg RNAi component, (2) 250 mg compound A daily and (3) NA. The patients were given three subcutaneous doses of the RNAi component on Day 1, 29 and 57. N represents the number of patients whose data was analyzed at the corresponding time.

FIGS. 5A-5C show the individual changes in HBsAg from Day 1 to Day 113 (FIG. 5A), Day 1 to Day 168 (FIG. 5B), or Day 1 to Day 336 (FIG. 5C) for CHB patients (cohort 12 of Example 1) receiving (1) 200 mg RNAi component, (2) 250 mg compound A daily and (3) NA. The patients were given three subcutaneous doses of the RNAi component on Day 1, 29 and 57. Each line represents one individual CHB patient.

FIG. 6 shows the mean HBsAg and log HBsAg changes from Day 1 to Day 113 for CHB patients (cohort 12 of Example 1) receiving (1) 200 mg RNAi component, (2) 250 mg compound A daily and (3) NA. The patients were given three subcutaneous doses of the RNAi component on Day 1, 29 and 57.

FIG. 7 shows individual reductions in HBsAg on Day 113 for CHB patients (cohort 12 of Example 1) receiving (1) 200 mg RNAi component, (2) 250 mg compound A daily and (3) NA. The patients were given three subcutaneous doses of the RNAi component on Day 1, 29 and 57. Each dot represents one individual CHB patient. Dots with an arrow indicate HBeAg positive patients. Dots without an arrow indicate HBeAg negative patients.

FIGS. 8A-8D show the individual changes in HBV DNA, HBV RNA, HBeAg and HBcrAg from Day 1 to Day 113 for CHB patients (cohort 12 of Example 1) receiving (1) 200 mg RNAi component, (2) 250 mg compound A daily and (3) NA. The patients were given three subcutaneous doses of the RNAi component on Day 1, 29 and 57. Each line represents one individual CHB patient.

FIG. 9 shows the mean HBsAg changes from Day 1 to Day 113 for CHB patients in cohorts 1b, 1c, 2b, 3b, 4b and 5b of Example 1, respectively. The patients were given three subcutaneous doses of the RNAi component in the amount of: 25 mg (cohort 1b), 50 mg (cohort 1c), 100 mg (cohort 2b), 200 mg (cohort 3b), 300 mg (cohort 4b) or 400 mg (cohort 5b) on Day 1, 29 and 57. All patients were given NA daily.

FIG. 10 shows the mean and individual HBsAg changes on Day 113 from Day 1 for all patients in cohorts 1b, 1c, 2b, 3b, 4b and 5b of Example 1. Dots with an arrow indicate HBeAg positive patients. Dots without an arrow indicate HBeAg negative patients.

FIGS. 11A-11D show the individual changes in HBV DNA, HBV RNA, HBeAg and HBcrAg from Day 1 to Day 113 for CHB patients in cohorts 1b, 1c, 2b, 3b, 4b and 5b of Example 1, respectively. The patients were given three subcutaneous doses of the RNAi component in the amount of: 25 mg (cohort 1b), 50 mg (cohort 1c), 100 mg (cohort 2b), 200 mg (cohort 3b), 300 mg (cohort 4b) or 400 mg (cohort 5b) on Day 1, 29 and 57. And all patients were given NA daily. Each line represents one individual CHB patient.

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

As used herein, the term “3-7 membered mono or polycyclic saturated ring” means saturated cyclic hydrocarbon with 3, 4, 5, 6 or 7 carbon atoms and is generic to cyclopropyl, cyclobutyl, cyclopentyl, cyclohexyl and cycloheptyl (monocyclic) and fused or spiro ring systems with 2 or more saturated rings with at most 7 carbon atoms (polycyclic). Such saturated ring optionally contains one or more heteroatoms, such that at least one carbon atom is replaced by a heteroatom selected from N, O and S, in particular from N and O. Examples include oxetane, tetrahydro-2H-pyranyl, piperidinyl, tetrahydro¬furanyl, morpholinyl, thiolane 1,1-dioxide and pyrrolidinyl. Preferred are saturated cyclic hydrocarbon with 3 or 4 carbon atoms and 1 oxygen atom. Examples include oxetane, and tetrahydrofuranyl.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl, oxo, and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

The terms “patient” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of the cardiac sarcomere. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyl-tartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

Positions indicated on phenyl (e.g. ortho, meta and/or para) are indicated relative to the bond connecting the phenyl to the main structure. An example with regard to the position of para R², location is indicated relative to the nitrogen (*) connected to the main structure:

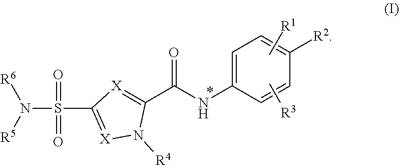

When any variable (e.g. halogen or C₁₋₄alkyl) occurs more than one time in any constituent, each definition is independent.

### Combination

Provided herein is a combination of an effective amount of an RNAi component and a capsid assembly modulator (CAM) or a pharmaceutically acceptable salt thereof.

### CAM Compound

In some embodiments, the CAM is a compound of Formula (I):

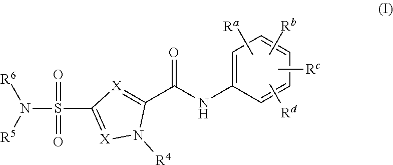

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments of compounds of Formula (I), R^{a}, R^{b} and R^{c} are independently hydrogen, fluoro, bromo, chloro, or CN. In some embodiments of compounds of Formula (I), R⁴ is C₁-C₃ alkyl. In some embodiments of compounds of Formula (I), R⁴ is methyl. In some embodiments of compounds of Formula (I), R⁶ is C₂-C₆ alkyl optionally substituted with one or more of —OH, fluoro, or C₁-C₄ alkyl optionally substituted with R¹⁰. In some embodiments of compounds of Formula (I), R⁶ is C₂-C₆ alkyl substituted with one or more fluoro. In some embodiments of compounds of Formula (I), each R⁷ is independent hydrogen, halogen or methyl. In some embodiments of compounds of Formula (I), at least one R⁷ is hydrogen.

In a further aspect, the CAM compound is a compound of Formula (IA)

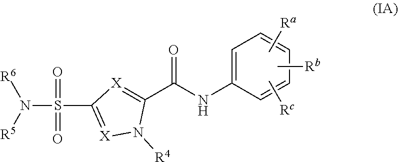

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, C₁-C₃alkyl and C₃-C₄ cycloalkyl;
R⁴ is Hydrogen, C₁-C₃alkyl or C₃-C₄cycloalkyl;
R⁵ is Hydrogen;
R⁶ is selected from the group consisting of C₂-C₆ alkyl, C₁-C₄ alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹ and C₁-C₄alkyl optionally substituted with R¹⁰;
R⁷ is hydrogen, —CN, Fluoro, Chloro, Bromo, —CHF₂, —CF₂-methyl, —CH₂F, —CF₃, C₁-C₃alkyl or C₃-C₄cycloalkyl;
R⁸ represents 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R′)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In one embodiment, the CAM compound is a compound Formula (IC)

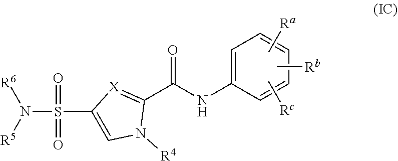

or a stereoisomer or tautomeric form thereof, wherein:
X is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, C₁-C₃alkyl and C₃-C₄cycloalkyl;
R⁴ is Hydrogen, C₁-C₃alkyl or C₃-C₄ cycloalkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄ alkyl optionally substituted with R¹⁰;
R⁷ is hydrogen, —CN, Fluoro, Chloro, Bromo, —CHF₂, —CF₂-methyl, —CH₂F, —CF₃, C₁-C₃alkyl or C₃-C₄cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments of compounds of Formula (IA) or (IC), R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN, and C₁-C₃alkyl. In some embodiments of compounds of Formula (IA) or (IC), R⁴ is Hydrogen, or methyl and R⁵ is Hydrogen. In some embodiments of compounds of Formula (IA) or (IC), R⁶ is selected from the group consisting of C₂-C₆alkyl and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰. In some embodiments of compounds of Formula (IA) or (IC), R⁷ is hydrogen, —CN, Fluoro, Chloro, Bromo, —CHF₂, —CF₂-methyl, —CH₂F, —CF₃ or methyl. In some embodiments of compounds of Formula (IA) or (IC), R⁹ is, C₁-C₄alkyloxy, —SO₂-methyl, —C(═O)—OR¹¹ or —C(═O)—N(R^{D1})₂. In some embodiments of compounds of Formula (IA) or (IC), R¹⁰ is —CN, —OH, Fluoro, —CHF₂, —CH₂F or —CF₃ and R¹¹ is hydrogen or C₁-C₃alkyl.

In some embodiments of compounds of Formula (IA) or (IC), R^{a}, R^{b} and R^{c} are independently hydrogen, fluoro, bromo, chloro, or CN. In some embodiments of compounds of Formula (IA) or (IC), R⁴ is C₁-C₃ alkyl. In some embodiments of compounds of Formula (IA) or (IC), R⁴ is methyl. In some embodiments of compounds of Formula (IA) or (IC), R⁶ is C₂-C₆ alkyl optionally substituted with one or more of —OH, fluoro, or C₁-C₄ alkyl optionally substituted with R¹⁰. In some embodiments of compounds of Formula (IA) or (IC), R⁶ is C₂-C₆ alkyl substituted with one or more fluoro. In some embodiments of compounds of Formula (IA) or (IC), each R⁷ is independent hydrogen, halogen or methyl. In some embodiments of compounds of Formula (IA) or (IC), at least one R⁷ is hydrogen.

Another embodiment of the present invention relates to those compounds of Formula (I), (IA), (IC) or any subgroup thereof as mentioned in any of the other embodiments wherein one or more of the following restrictions apply:
(a) R⁴ is C₁-C₃alkyl, preferably methyl; R⁶ is selected from the group consisting of C₂-C₆alkyl optionally being substituted with one or more Fluoro; and R⁷ is hydrogen Fluoro, Chloro or C₁-C₃alkyl, preferably hydrogen Fluoro, Chloro or methyl.
(b) R^{b} is Hydrogen or Fluoro.
(c) R^{a} and R^{c} are independently selected from the group consisting of Hydrogen, Fluoro, Chloro —CN and methyl.
(d) R^{b} is Hydrogen or Fluoro and R^{a} and R^{c} are independently selected from the group consisting of Hydrogen, Fluoro, Chloro and —CN.
(e) R⁶ contains a 3-7 membered saturated ring optionally containing one oxygen, more specifically R⁶ is a 4 or 5 membered saturated ring containing one oxygen, such 4 or 5 membered saturated ring optionally substituted with C₁-C₄alkyl optionally substituted with R¹⁰.
(f) R⁶ comprises a branched C₃-C₆alkyl optionally substituted with one or more Fluoro, or wherein R⁶ comprises a C₃-C₆cycloalkyl wherein such C₃-C₆cycloalkyl is substituted with one or more Fluoro or substituted with C₁-C₄alkyl substituted with one or more Fluoro, or wherein R⁶ comprises a C₃-C₆cycloalkyl optionally substituted with one or more Fluoro and/or substituted with C₁-C₄alkyl optionally substituted with one or more Fluoro.
(g) R⁶ comprises a branched C₃-C₆alkyl optionally substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. More specifically, R⁶ is a branched C₃-C₆alkyl substituted with one or more Fluoro.
(h) R⁴ is C₁-C₃alkyl, preferably methyl; R⁶ is selected from the group consisting of C₂-C₆alkyl optionally being substituted with one or more Fluoro; and R⁷ is hydrogen, Fluoro, Chloro or C₁-C₃alkyl, preferably hydrogen Fluoro, Chloro or methyl.

In some embodiments, the compound of Formula (IA) is:

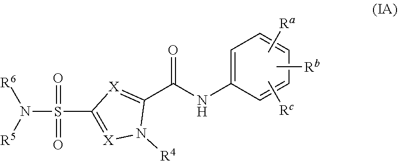

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, CN, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments, the compound of Formula (IA) is:

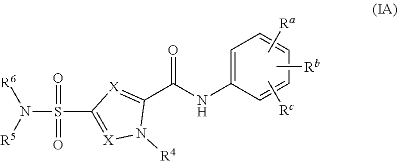

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In a further aspect, the CAM compound is a compound of Formula (I) is:

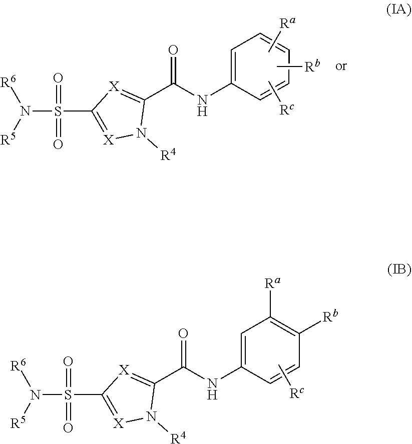

or a stereoisomer or tautomeric form thereof, wherein:
Each X independently is CR⁷;
R^{a}, R^{b} and R^{c} are independently selected from the group consisting of Hydrogen, Fluoro, Bromo, Chloro, —CHF₂, —CF₂-methyl, —CH₂F, —CF₃, —OCF₃, —CN and methyl;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered mono or polycyclic saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹⁰ and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments of compounds of Formula (I), (IA), (IB) or (IC), R⁴ is methyl. In some embodiments of compounds of Formula (I), (IA), (IB) or (IC), R^{b} is Hydrogen or Fluoro. In some embodiments of compounds of Formula (I), (IA), (IB) or (IC), R^{a} and R^{c} are independently selected from the group consisting of Hydrogen, Fluoro, Chloro —CN and methyl. Preferably, R^{b} is Hydrogen or Fluoro and R^{a} and R^{c} are independently selected from the group consisting of Hydrogen, Fluoro, Chloro and —CN.

In some embodiments of compounds of Formula (I), (IA), (IB) or (IC), R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, —OH, Fluoro, oxo, R⁹, R¹ and C₁-C₄alkyl optionally substituted with R¹⁰. In some embodiments of compounds of Formula (I), (IA), (IB) or (IC), R⁶ contains a 3-7 membered saturated ring optionally containing one oxygen, more specifically R⁶ is a 5 membered saturated ring containing one oxygen, such 5 membered saturated ring optionally substituted with C₁-C₄alkyl optionally substituted with R¹⁰.

In some embodiments of compounds of Formula (I), (IA), (IB) or (IC), R⁶ comprises a branched C₃-C₆alkyl optionally substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. In some embodiments of compounds of Formula (I), (IA), (IB) or (IC), R⁶ is a branched C₃-C₆alkyl substituted with one or more Fluoro.

In some embodiments of compounds of Formula (I), (IA), (IB) or (IC), R^{a}, R^{b} and R^{c} are independently hydrogen, fluoro, bromo, chloro, or CN. In some embodiments of compounds of Formula (I), (IA), (IB) or (IC), R⁴ is C₁-C₃ alkyl. In some embodiments of compounds of Formula (I), (IA), (IB) or (IC), R⁴ is methyl. In some embodiments of compounds of Formula (I), (IA), (IB) or (IC), R⁶ is C₂-C₆ alkyl optionally substituted with one or more of —OH, fluoro, or C₁-C₄ alkyl optionally substituted with R¹⁰. In some embodiments of compounds of Formula (I), (IA), (IB) or (IC), R⁶ is C₂-C₆ alkyl substituted with one or more fluoro. In some embodiments of compounds of Formula (I), (IA), (IB) or (IC), each R⁷ is independent hydrogen, halogen or methyl. In some embodiments of compounds of Formula (IA) or (IC), at least one R⁷ is hydrogen.

In a further aspect, the CAM compound is a compound of Formula (ID)

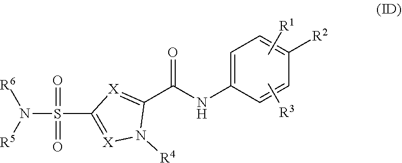

or a stereoisomer or tautomeric form thereof, wherein:
Each X independently is CR⁷;
R² is Hydrogen, CN, Chloro or Fluoro;
R¹ and R³ are independently selected from the group consisting of Hydrogen, Fluoro, Bromo, Chloro, —CHF₂, —CH₂F, —CF₃, —OCF₃, —CN and methyl, wherein at maximum one of R¹ R² and R³ is Hydrogen if one of R¹ and R³ is Chloro or —OCF₃;
R⁴ is Hydrogen or C₁-C₃alkyl;
R⁵ is Hydrogen;
R⁶ is selected from the group consisting of C₂-C₆alkyl, C₁-C₄alkyl-R⁸ optionally substituted with one or more Fluoro, C₁-C₄alkyl-R⁹ optionally substituted with one or more Fluoro, and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is, C₁-C₄alkyloxy, —SO₂-methyl, —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salt or a solvate thereof.

In some embodiments, the compound of Formula (ID) is:

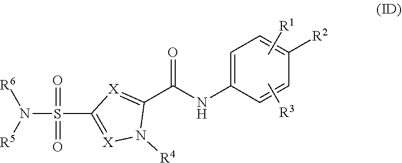

or a stereoisomer or tautomeric form thereof, wherein:
Each X independently is CR⁷;
R² is Hydrogen, CN, or Fluoro;
R¹ and R³ are independently selected from the group consisting of Hydrogen, Fluoro, Bromo, Chloro, —CHF₂, —CH₂F, —CF₃, —CN and methyl, wherein at maximum one of R¹, R² and R³ is Hydrogen if one of R¹ and R³ is Chloro;
R⁴ is Hydrogen or C₁-C₃alkyl;
R⁵ is Hydrogen;
R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is —C(═O)—OR¹¹ or —C(═O)—N(R¹¹)₂;
R¹⁰ is —CN, —OH, Fluoro, —CHF₂, —CH₂F or —CF₃; and
R¹¹ is hydrogen or C₁-C₃alkyl.

In some embodiments, the compound of Formula (ID) is:

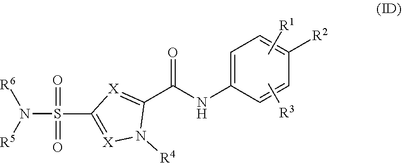

or a stereoisomer or tautomeric form thereof, wherein:
Each X independently is CR⁷;
R² is Hydrogen or Fluoro;
R¹ and R³ are independently selected from the group consisting of Hydrogen, Fluoro, Bromo, Chloro, CHF₂, CH₂F, CF₃ and methyl, wherein at maximum one of R¹, R² and R³ is Hydrogen;
R⁴ is Hydrogen or methyl;
R⁵ is Hydrogen;
R⁶ is selected from the group consisting of C₂-C₆alkyl, C₁-C₃alkyl-R⁸ and a 3-7 membered saturated ring optionally containing one or more heteroatoms each independently selected from the group consisting of O, S and N, such 3-7 membered saturated ring or C₂-C₆alkyl optionally being substituted with one or more substituents each independently selected from the group consisting of Hydrogen, OH, Fluoro, and C₁-C₄alkyl;
R⁷ is Hydrogen, methyl, Fluoro or Chloro;
R⁸ is 3-7 membered saturated ring optionally containing one or more heteroatoms each independently selected from the group consisting of O, S and N.

In some embodiments of compounds of Formula (ID), at least one X is CH. In some embodiments of compounds of Formula (ID), R^{a}, R^{b} and R^{c} are independently hydrogen, fluoro, bromo, chloro, or CN. In some embodiments of compounds of Formula (ID), R⁴ is C₁-C₃ alkyl. In some embodiments of compounds of Formula (ID), R⁴ is methyl. In some embodiments of compounds of Formula (ID), R⁶ is C₂-C₆ alkyl optionally substituted with one or more of —OH, fluoro, or C₁-C₄ alkyl optionally substituted with R¹⁰. In some embodiments of compounds of Formula (ID), R⁶ is C₂-C₆ alkyl substituted with one or more fluoro. In some embodiments of compounds of Formula (ID), each R⁷ is independent hydrogen, halogen or methyl. In some embodiments of compounds of Formula (ID), at least one R⁷ is hydrogen.

In another aspect, the CAM compound is a compound of Formula (Ia):

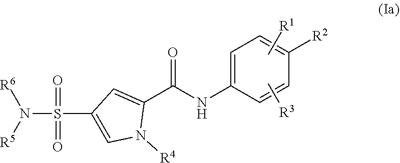

or a stereoisomer or tautomeric form thereof, wherein:
R² is Hydrogen, CN or Fluoro;
R¹ and R³ are independently selected from the group consisting of Hydrogen, Fluoro, Bromo, Chloro, —CHF₂, —CH₂F, —CF₃, —CN and methyl, wherein at maximum one of R¹, R² and R³ is Hydrogen if one of R¹ and R³ is Chloro;
R⁴ is Hydrogen or C₁-C₃alkyl;
R⁵ is Hydrogen;
R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰;
R⁷ is hydrogen, methyl, Fluoro or Chloro;
R⁸ is 3-7 membered saturated ring optionally containing one or more heteroatoms each independently selected from the group consisting of O, S and N, such 3-7 membered saturated ring optionally being substituted with one or more C₁-C₄alkyl optionally substituted with R¹⁰;
R⁹ is —C(═O)—OR¹¹ or —C(═O)—N(R¹¹)₂;
R¹⁰ is —CN, —OH, Fluoro, —CHF₂, —CH₂F or —CF₃;
R¹¹ is hydrogen or C₁-C₃alkyl;
or a pharmaceutically acceptable salts or a solvate thereof.

In some embodiments, compounds of Formula (ID) are:

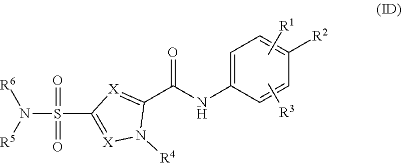

or a stereoisomer or tautomeric form thereof, wherein:
R² is Hydrogen or Fluoro;
R¹ and R³ are independently selected from the group consisting of Hydrogen, Fluoro, CHF₂, CH₂F, CF₃ and methyl, wherein at maximum one of R¹, R² and R³ is Hydrogen;
R⁴ is Hydrogen or methyl;
R⁵ is Hydrogen;
R⁶ is selected from the group consisting of C₂-C₆ alkyl and a 3-7 membered saturated ring optionally containing one or more heteroatoms each independently selected from the group consisting of, O, S and N, such 3-7 membered saturated ring or C₁-C₆alkyl optionally being substituted with one or more substituents each independently selected from the group consisting of Hydrogen, OH and C₁-C₄ alkyl.

In another aspect, the CAM compound is a compound of Formula (II):

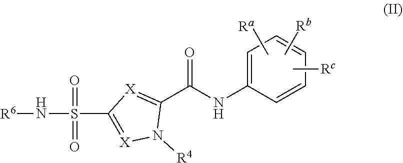

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

In some embodiments of compounds of Formula (II), R^{a}, R^{b} and R^{c} are independently hydrogen, fluoro, bromo, chloro, or CN. In some embodiments of compounds of Formula (II), R⁴ is C₁-C₃ alkyl. In some embodiments of compounds of Formula (II), R⁴ is methyl. In some embodiments of compounds of Formula (II), R⁶ is C₂-C₆ alkyl optionally substituted with one or more of —OH, fluoro, or C₁-C₄ alkyl optionally substituted with R¹⁰. In some embodiments of compounds of Formula (II), R⁶ is C₂-C₆ alkyl substituted with one or more fluoro. In some embodiments of compounds of Formula (II), each R⁷ is independent hydrogen, halogen or methyl. In some embodiments of compounds of Formula (II), at least one R⁷ is hydrogen.

In some embodiments, the compound of Formula (ID) is a compound of Formula (III):

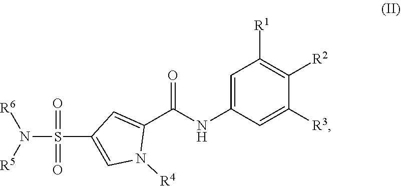

wherein R¹-R⁶ are as defined in Formula (ID).

In some embodiments, the compound of Formula (ID) is a compound of Formula (IV):

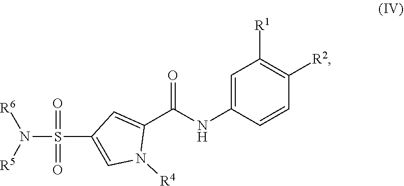

wherein R¹, R², R⁴-R⁶ are as defined in Formula (ID).

In some embodiments of compounds of Formula (III) and (IV), R² is Hydrogen, CN or Fluoro. In some embodiments of compounds of Formula (III) and (IV), R¹ is independently selected from the group consisting of Fluoro, Bromo, Chloro, —CHF₂, —CH₂F, —CF₃, —CN and methyl, wherein if R¹ is Chloro, R² is not Hydrogen. In some embodiments of compounds of Formula (III) and (IV), R⁴ is Hydrogen or C₁-C₃alkyl; R⁵ is Hydrogen; and R⁶ is selected from the group consisting of C₂-C₆alkyl, C₁-C₄alkyl-R⁸, C₁-C₄alkyl-R⁹ and a 3-7 membered saturated ring optionally containing one or more heteroatoms each independently selected from the group consisting of O, S and N, such 3-7 membered saturated ring or —C₂-C₆alkyl optionally being substituted with one or more substituents each independently selected from the group consisting of Hydrogen, —OH, Fluoro, oxo and C₁-C₄alkyl optionally substituted with R¹⁰. In some embodiments of compounds of Formula (III) and (IV), R⁷ is methyl, Fluoro or Chloro. In some embodiments of compounds of Formula (III) and (IV), R is 3-7 membered saturated ring optionally containing one or more heteroatoms each independently selected from the group consisting of O, S and N. In some embodiments of compounds of Formula (III) and (IV), R⁹ is —C(═O)—OR¹¹ or —C(═O)—N(R^{D1})₂. In some embodiments of compounds of Formula (III) and (IV), R¹⁰ is —CN, Fluoro, —CHF₂, —CH₂F or —CF₃. In some embodiments of compounds of Formula (III) and (IV), R¹¹ is hydrogen.

In some embodiments of compounds of Formula (I)-(IV), (Ia), (IB), (IC) or (ID), R¹ is selected from either Bromo, Chloro, Fluoro or methyl, or Fluoro or methyl. In some embodiments of compounds of Formula (I)-(IV), (Ia), (IB), (IC) or (ID), R¹ is selected from either Fluoro or methyl and at least one of R¹ and R³ is Fluoro. In some embodiments of compounds of Formula (I)-(IV), (Ia), (IB), (IC) or (ID), R¹ is selected from either Fluoro or methyl and at least one of R¹ and R³ is Fluoro, and the other R¹ or R³ is selected from methyl, Fluoro, CHF₂, CH₂F, CF₃ and methyl. In some embodiments of compounds of Formula (I)-(IV), (Ia), (IB), (IC) or (ID), at least two of R¹, R² and R³ are halogens, preferably Bromo, Fluoro or Chloro, even more preferably Fluoro or Chloro. In some embodiments of compounds of Formula (I)-(IV), (Ia), (IB), (IC) or (ID), each of R¹, R² and R³ are halogen, preferably Bromo, Fluoro or Chloro, even more preferably Fluoro or Chloro.

In some embodiments of compounds of Formula (I)-(IV), (Ia), (IB), (IC) or (ID), R⁴ is methyl or ethyl, preferably methyl. In some embodiments of compounds of Formula (I)-(IV), (Ia), (IB), (IC) or (ID), R⁶ contains a 3-7 membered saturated ring optionally containing one oxygen, preferably R⁶ is a 5 membered saturated ring containing one oxygen. In some embodiments of compounds of Formula (I)-(IV), (Ia), (IB), (IC) or (ID), R⁶ comprises a C₁-C₄alkyl substituted with one or more Fluoro. In some embodiments of compounds of Formula (I)-(IV), (Ia), (IB), (IC) or (ID), R⁶ comprises a branched C₃-C₆alkyl substituted with one or more Fluoro, or R⁶ comprises a C₃-C₆cycloalkyl wherein such C₃-C₆cycloalkyl is substituted with one or more Fluoro or substituted with C₁-C₄ substituted with one or more Fluoro. In some embodiments of compounds of Formula (I)-(IV), (Ia), (IB), (IC) or (ID), R⁶ comprises a carbon atom without hydrogen substituent. Preferably, carbon without hydrogen substituent is directly attached to the Nitrogen of the —N—SO₂˜ moiety.

In some embodiments of compounds of Formula (I)-(IV), (Ia), (IB), (IC) or (ID), R^{a}, R^{b} and R^{c} are independently hydrogen, fluoro, bromo, chloro, or CN. In some embodiments of compounds of Formula (I)-(IV), (Ia), (IB), (IC) or (ID), R⁴ is C₁-C₃ alkyl. In some embodiments of compounds of Formula (I)-(IV), (Ia), (IB), (IC) or (ID), R⁴ is methyl. In some embodiments of compounds of Formula (I)-(IV), (Ia), (IB), (IC) or (ID), R⁶ is C₂-C₆ alkyl optionally substituted with one or more of —OH, fluoro, or C₁-C₄ alkyl optionally substituted with R¹⁰. In some embodiments of compounds of Formula (I)-(IV), (Ia), (IB), (IC) or (ID), R⁶ is C₂-C₆ alkyl substituted with one or more fluoro. In some embodiments of compounds of Formula (I)-(IV), (Ia), (IB), (IC) or (ID), each R⁷ is independent hydrogen, halogen or methyl. In some embodiments of compounds of Formula (I)-(IV), (Ia), (IB), (IC) or (ID), at least one R⁷ is hydrogen.

Further combinations of any of the embodiments are also envisioned to be in the scope of the present invention.

In another aspect, the CAM compound is selected from the group consisting of:

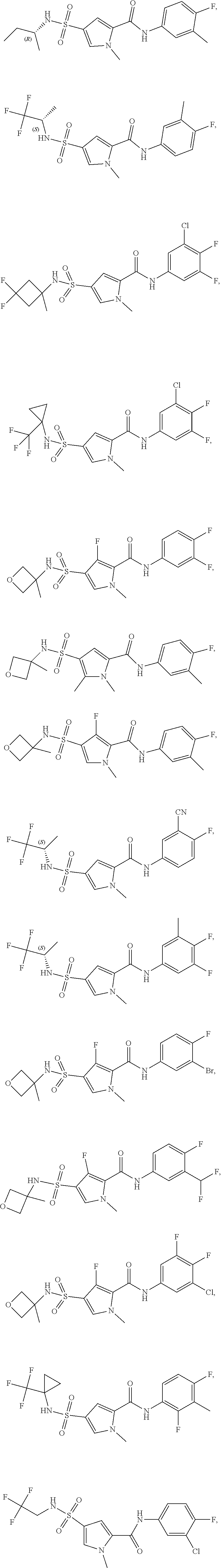

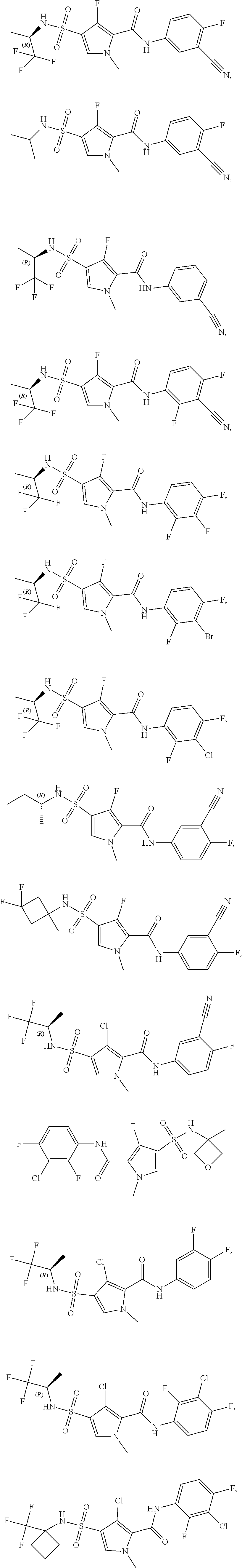

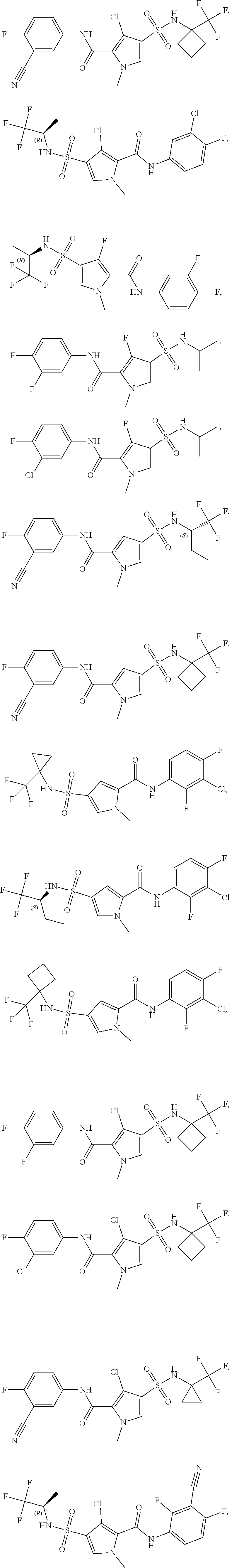

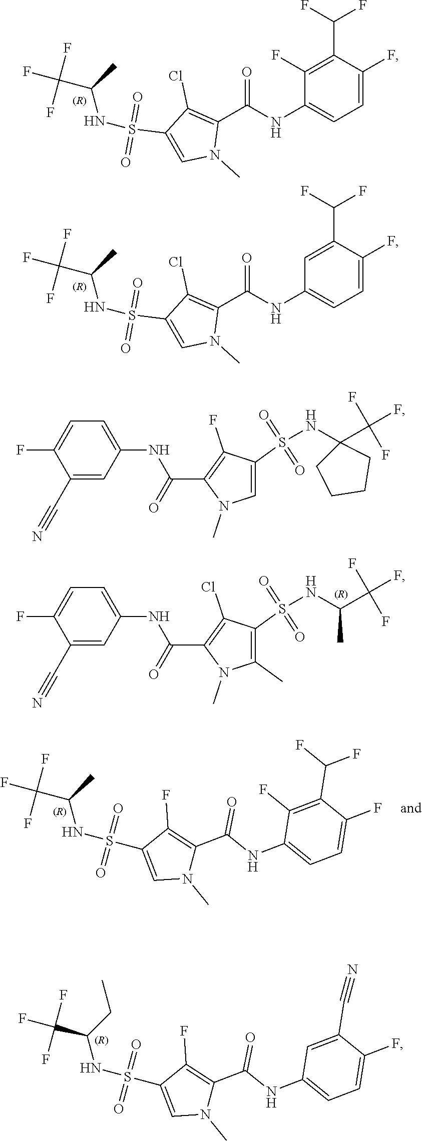

or a pharmaceutically acceptable salt of any of the foregoing.

In another aspect, the CAM compounds of the present disclosure or any subgroup thereof may be in the form of a pharmaceutically acceptable salt or a solvate. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

### RNAi Component

In one aspect, the RNAi component comprises one or more RNAi agents. Each RNAi agent disclosed herein includes at least a sense strand and an antisense strand. The sense strand and the antisense strand can be partially, substantially, or fully complementary to each other. The length of the RNAi agent sense and antisense strands described herein each can be 16 to 30 nucleotides in length. In some embodiments, the sense and antisense strands are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The HBV RNAi agents disclosed herein have been designed to include antisense strand sequences that are at least partially complementary to a sequence in the HBV genome that is conserved across the majority of known serotypes of HBV. The RNAi agents described herein, upon delivery to a cell expressing HBV, inhibit the expression of one or more HBV genes in vivo or in vitro.

An RNAi agent includes a sense strand (also referred to as a passenger strand) that includes a first sequence, and an antisense strand (also referred to as a guide strand) that includes a second sequence. A sense strand of the HBV RNAi agents described herein includes a core stretch having at least about 85% identity to a nucleotide sequence of at least 16 consecutive nucleotides in an HBV mRNA. In some embodiments, the sense strand core nucleotide stretch having at least about 85% identity to a sequence in an HBV mRNA is 16, 17, 18, 19, 20, 21, 22, or 23 nucleotides in length. An antisense strand of an HBV RNAi agent comprises a nucleotide sequence having at least about 85% complementary over a core stretch of at least 16 consecutive nucleotides to a sequence in an HBV mRNA and the corresponding sense strand. In some embodiments, the antisense strand core nucleotide sequence having at least about 85% complementarity to a sequence in an HBV mRNA or the corresponding sense strand is 16, 17, 18, 19, 20, 21, 22, or 23 nucleotides in length.

In some embodiments, the RNAi component comprises a first RNAi agent comprising an antisense strand comprising a nucleotide sequence of any one of the following: SEQ ID NO:1, SEQ ID NO:2, SEQ ID NO:3, SEQ ID NO: 4, SEQ ID NO: 5, SEQ ID NO:6, and SEQ ID NO:7, and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15, or a second RNAi agent comprising an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19. In some embodiments, the RNAi component comprises a first RNAi agent comprising an antisense strand comprising a nucleotide sequence of any one of the following: SEQ ID NO:1, SEQ ID NO:2, SEQ ID NO:3, SEQ ID NO: 4, SEQ ID NO: 5, SEQ ID NO:6, and SEQ ID NO:7, and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15, and a second RNAi agent comprising an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19.

In some embodiments, the first and the second RNAi agents disclosed herein comprise any of the sequences in Table 1.

TABLE 1
Exemplary sequences for first and
second RNAi agents

| Antisense | | Sense | |
|---|---|---|---|
| SEQ | | SEQ | |
| ID | Unmodified sequence | ID | Unmodified sequence |
| NO | (5′ → 3′) | NO | (5′ → 3′) |
| 5 | AGAAAAUUGAGAGAAGUCCAC | 14 | GUGGACUUCUCUCAAUUUUCU |
| 6 | AGAAAAUUGAGAGAAGUCCAC | 14 | GUGGACUUCUCUCAAUUUUCU |
| | UU | | |
| 7 | AGAAAAUUGAGAGAAGUCCAC | 15 | GGUGGACUUCUCUCAAUUUUC |
| | C | | U |
| 9 | UACCAAUUUAUGCCUACAGCG | 19 | CGCUGUAGGCAUAAAUUGGUA |

### Targeting Group

In some embodiments, the RNAi agents are delivered to target cells or tissues using any oligonucleotide delivery technology known in the art. Nucleic acid delivery methods include, but are not limited to, by encapsulation in liposomes, by iontophoresis, or by incorporation into other vehicles, such as hydrogels, cyclodextrins, biodegradable nanocapsules, and bioadhesive microspheres, proteinaceous vectors or Dynamic Polyconjugates (DPCs) (see, for example WO 2000/053722, WO 2008/0022309, WO 2011/104169, and WO 2012/083185, each of which is incorporated herein by reference). In some embodiments, an HBV RNAi agent is delivered to target cells or tissues by covalently linking the RNAi agent to a targeting group. In some embodiments, the targeting group can include a cell receptor ligand, such as an asialoglycoprotein receptor (ASGPr) ligand. In some embodiments, an ASGPr ligand includes or consists of a galactose derivative cluster. In some embodiments, a galactose derivative cluster includes an N-acetyl-galactosamine trimer or an N-acetyl-galactosamine tetramer. In some embodiments, a galactose derivative cluster is an N-acetyl-galactosamine trimer or an N-acetyl-galactosamine tetramer.

A targeting group can be linked to the 3′ or 5′ end of a sense strand or an antisense strand of an HBV RNAi agent. In some embodiments, a targeting group is linked to the 3′ or 5′ end of the sense strand. In some embodiments, a targeting group is linked to the 5′ end of the sense strand. In some embodiments, a targeting group is linked to the RNAi agent via a linker.

In some embodiments, the RNAi component comprises a combination or cocktail of a first and a second RNAi agents having different nucleotide sequences. In some embodiments, the first and the second RNAi agents are each separately and independently linked to targeting groups. In some embodiments, the first and the second RNAi agents are each linked to targeting groups comprised of N-acetyl-galactosamines. In some embodiments, when first and the second RNAi agents are included in a composition, each of the RNAi agents is linked to the same targeting group. In some embodiments, when first and the second RNAi agents are included in a composition, each of the RNAi agents is linked to different targeting groups, such as targeting groups having different chemical structures.

In some embodiments, targeting groups are linked to the first and the second RNAi agents without the use of an additional linker. In some embodiments, the targeting group is designed having a linker readily present to facilitate the linkage to the first or the second RNAi agent. In some embodiments, when the first and the second RNAi agents are included in a composition, the first and the second RNAi agents may be linked to the targeting groups using the same linkers. In some embodiments, when the first and the second RNAi agents are included in a composition, the first and the second RNAi agents are linked to the targeting groups using different linkers.

Examples of targeting groups and linking groups are provided in Table 2. The non-nucleotide group can be covalently linked to the 3′ and/or 5′ end of either the sense strand and/or the antisense strand. In some embodiments, the first or second RNAi agent contains a non-nucleotide group linked to the 3′ and/or 5′ end of the sense strand. In some embodiments, a non-nucleotide group is linked to the 5′ end of the first or second RNAi agent sense strand. A non-nucleotide group may be linked directly or indirectly to the first or second RNAi agent via a linker/linking group. In some embodiments, a non-nucleotide group is linked to the first or second RNAi agent via a labile, cleavable, or reversible bond or linker.

Targeting groups and linking groups include the following, for which their chemical structures are provided below in Table 2: (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s. Each sense strand and/or antisense strand can have any targeting groups or linking groups listed above, as well as other targeting or linking groups, conjugated to the 5′ and/or 3′ end of the sequence.

TABLE 2
Structures Representing Various Modified Nucleotides, Targeting Groups,
And Linking Groups

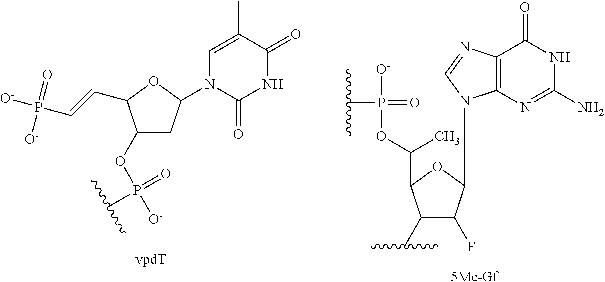

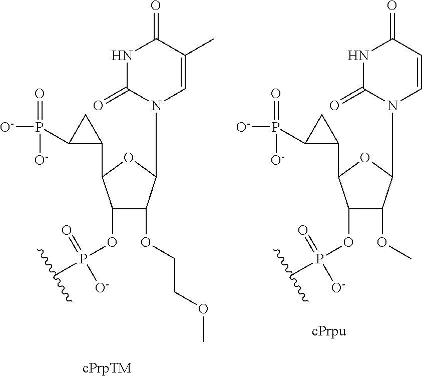 [SMILES: *P(=O)([O-])OC1C(C2CC2P(=O)([O-])[O-])OC(n2cc(C)c(=O)[nH]c2=O)C1OCCOC ; *P(=O)([O-])OC1C(C2CC2P(=O)([O-])[O-])OC(n2ccc(=O)[nH]c2=O)C1OC]

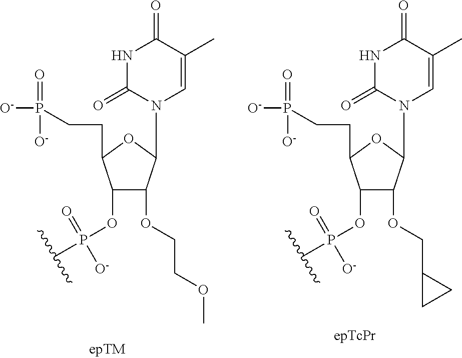 [SMILES: *P(=O)([O-])OC1C(CCP(=O)([O-])[O-])OC(n2cc(C)c(=O)[nH]c2=O)C1OCCOC ; *P(=O)([O-])OC1C(CCP(=O)([O-])[O-])OC(n2cc(C)c(=O)[nH]c2=O)C1OCC1CC1]

When positioned internally on oligonucleotide:

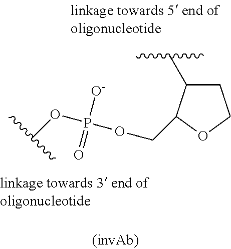 [SMILES: *OP(=O)([O-])OCC1OCCC1*]

When positioned internally on oligonucleotide:

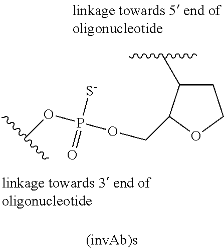 [SMILES: *OP(=O)([S-])OCC1OCCC1*]

When positioned at the 3′ terminal end of oligonucleotide:

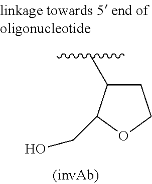 [SMILES: *C1CCOC1CO]

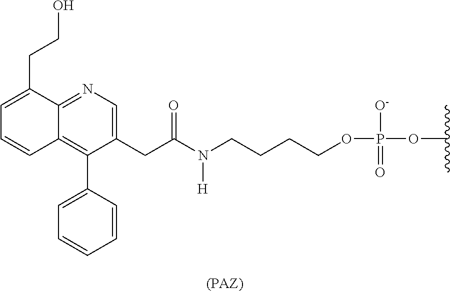 [SMILES: *OP(=O)([O-])OCCCCNC(=O)Cc1cnc2c(CCO)cccc2c1-c1ccccc1 ; "(PAZ)"]

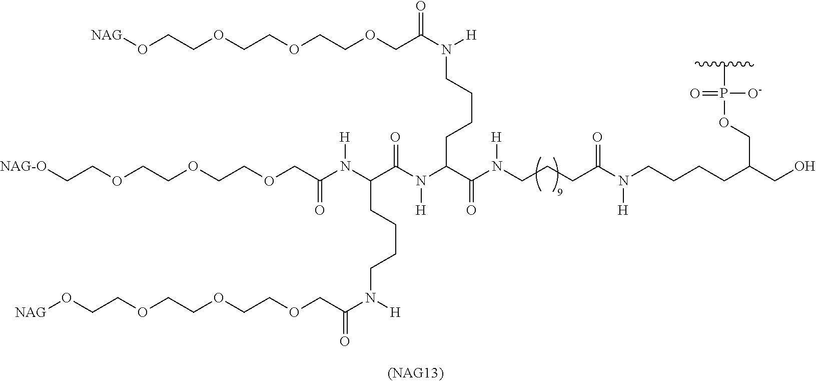

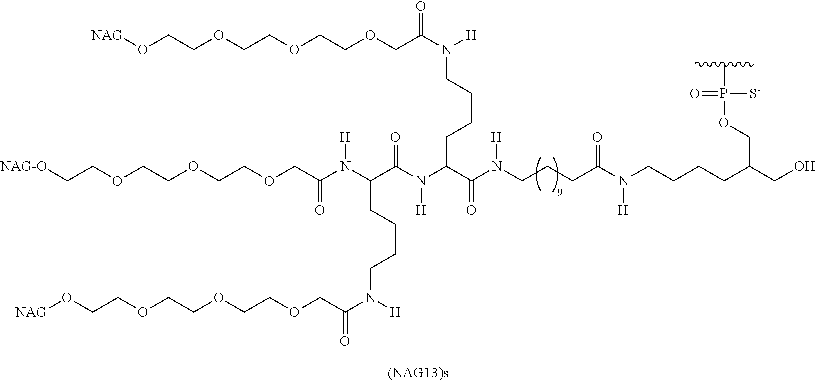

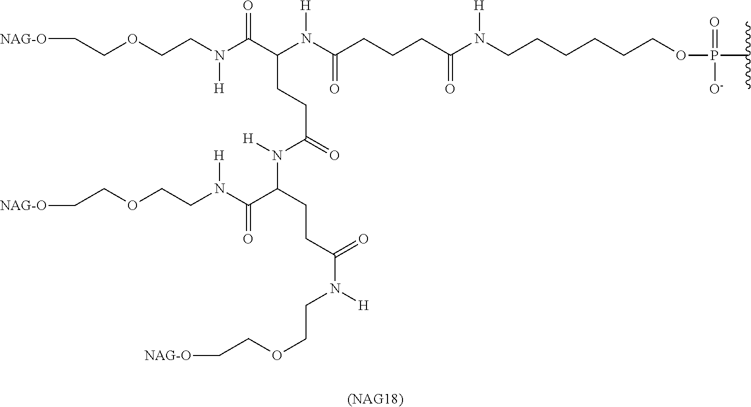

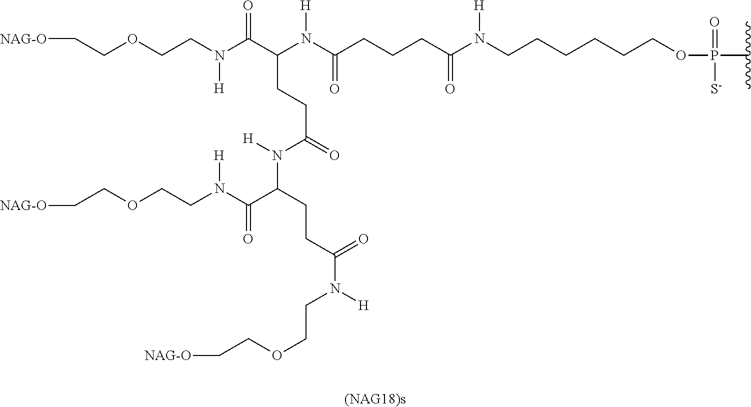

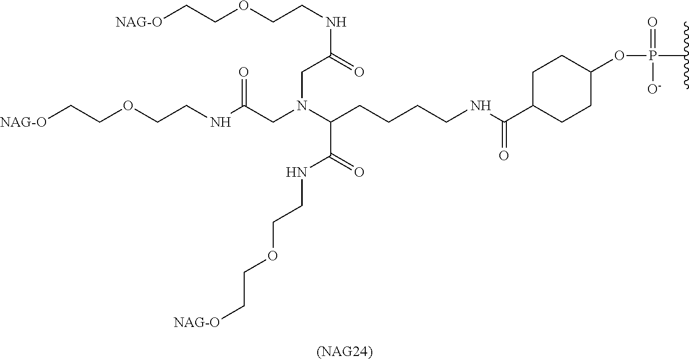

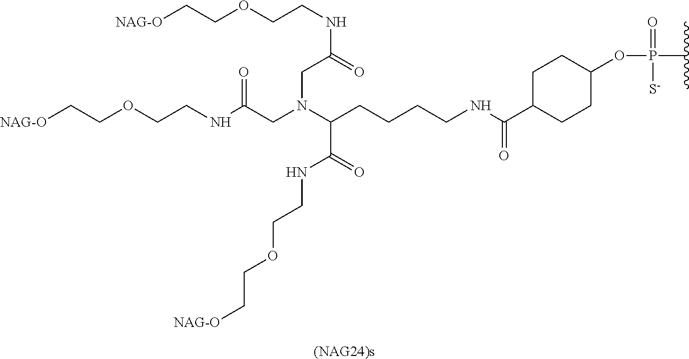

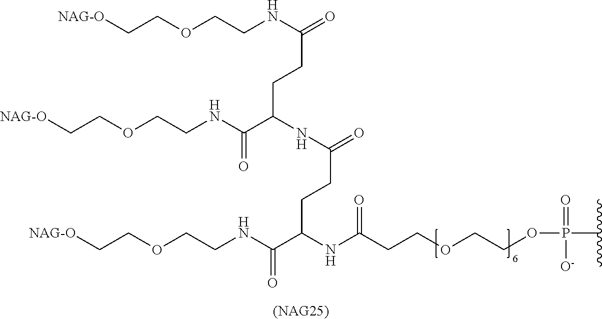

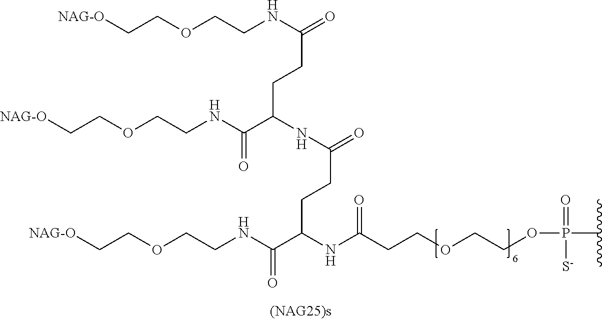

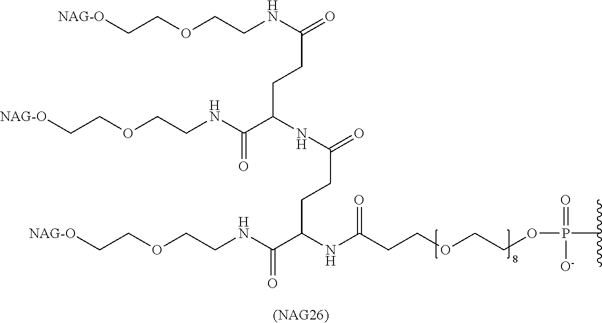

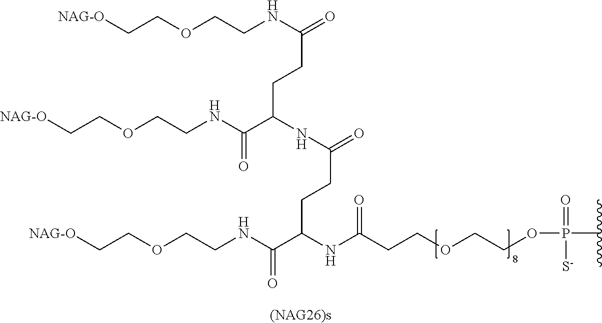

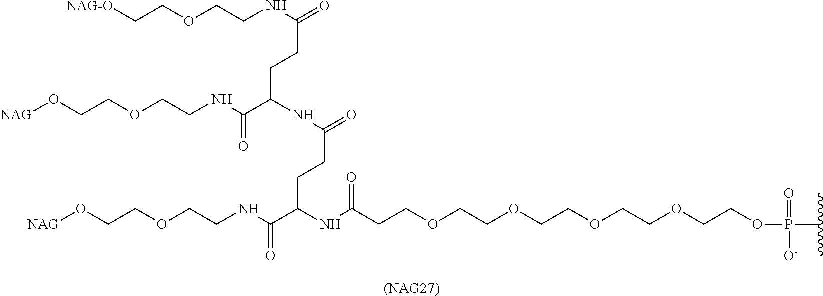

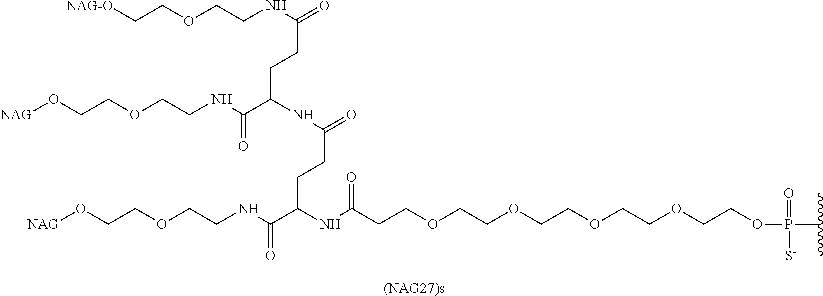

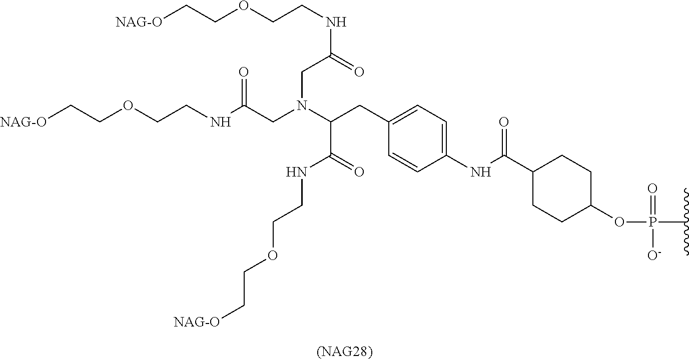

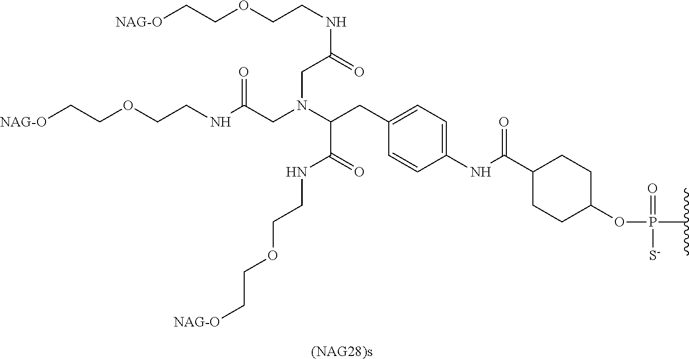

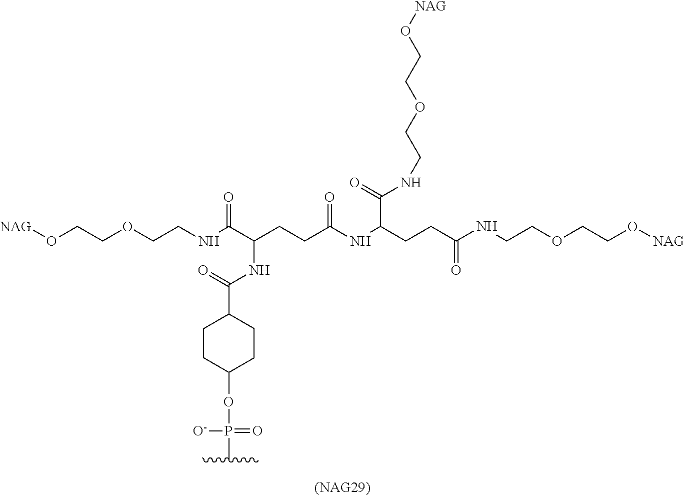

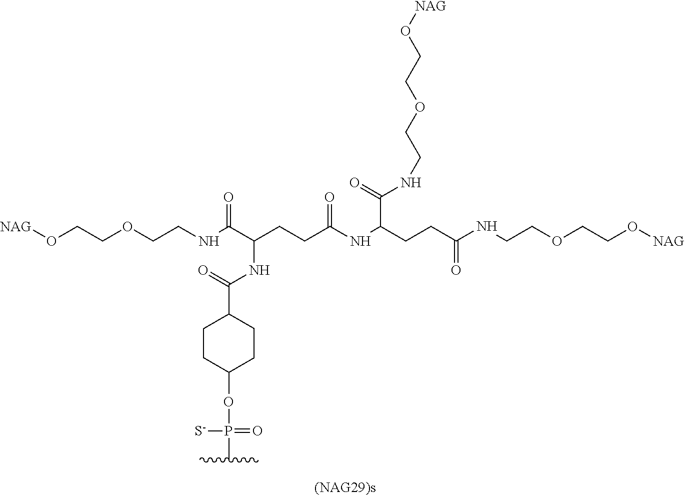

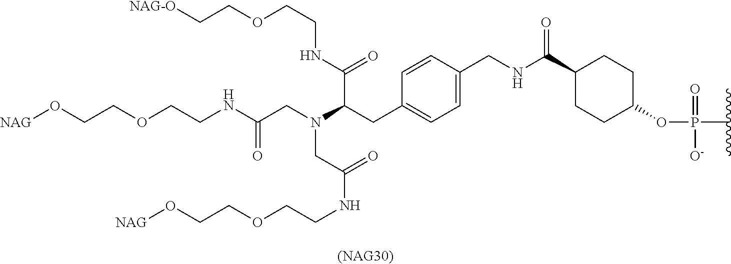

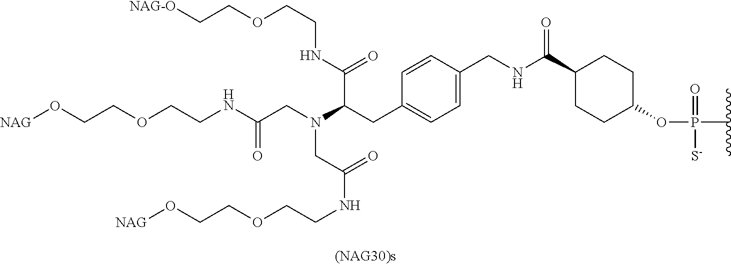

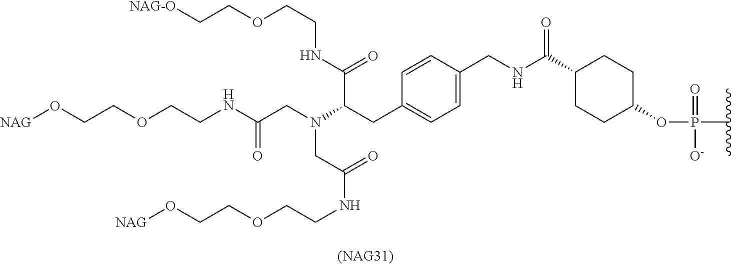

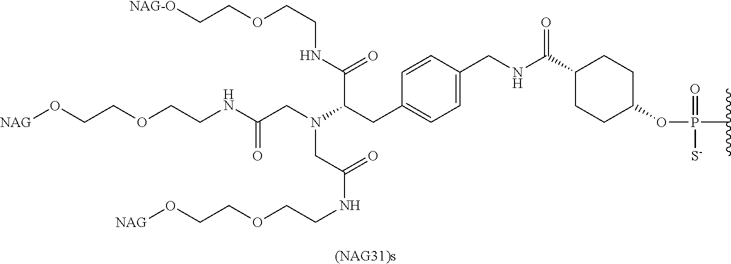

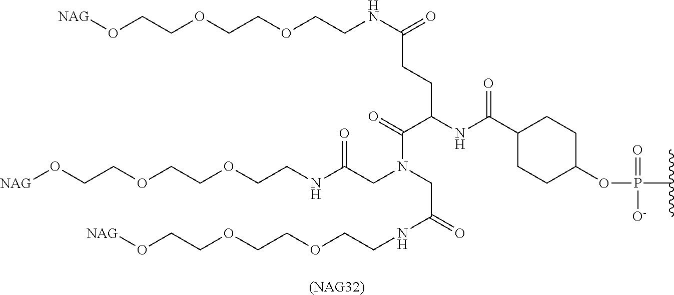

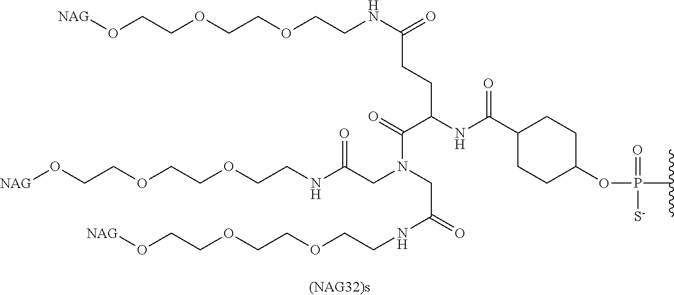

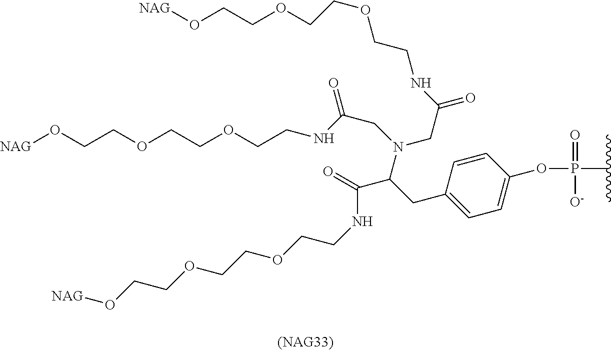

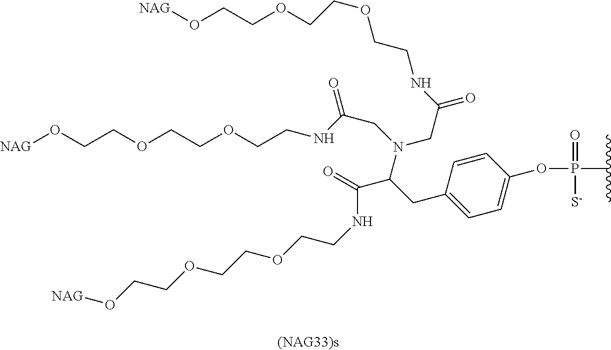

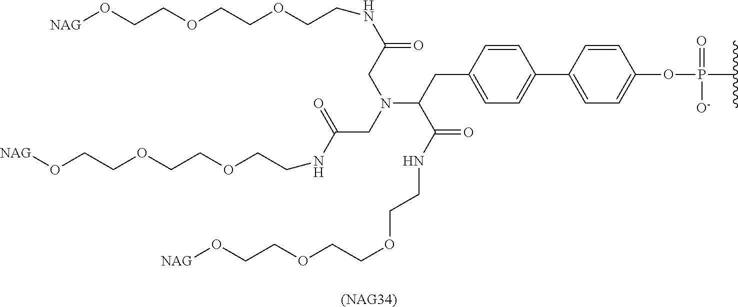

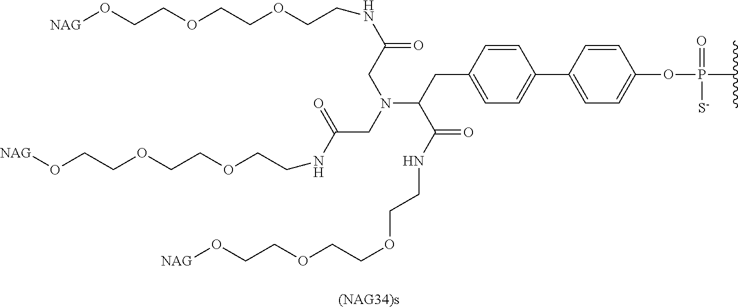

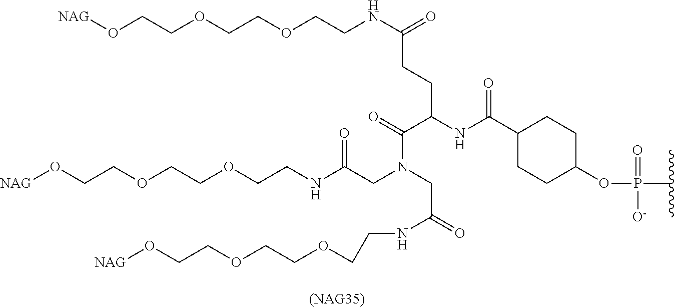

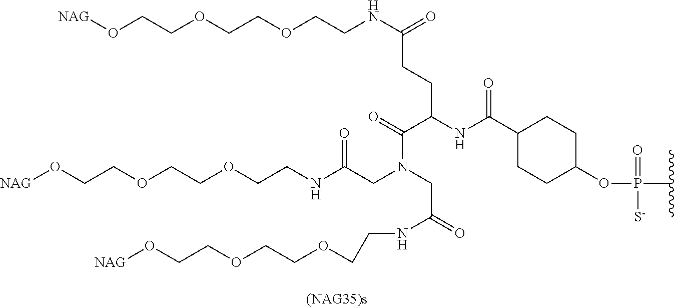

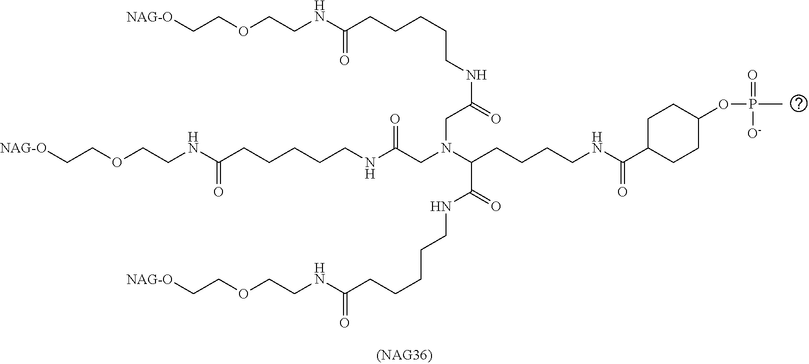

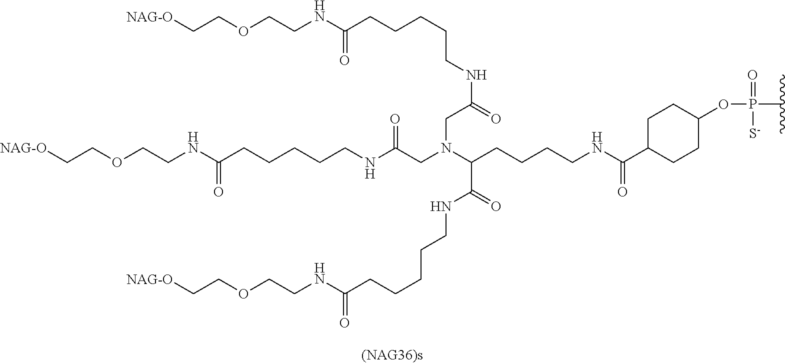

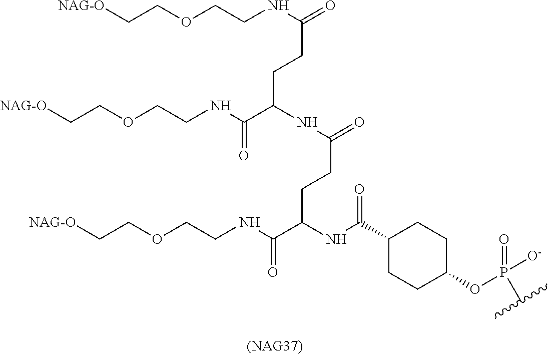

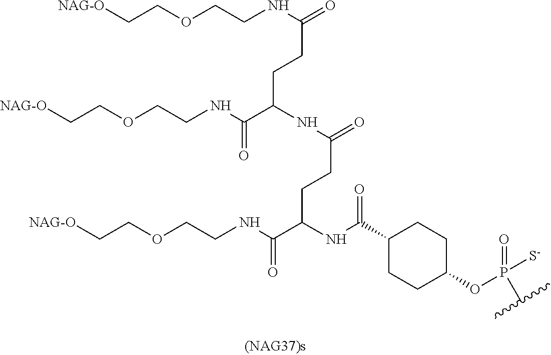

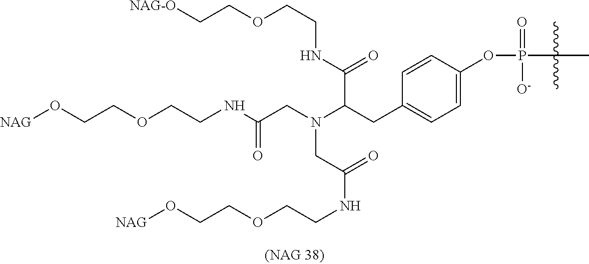

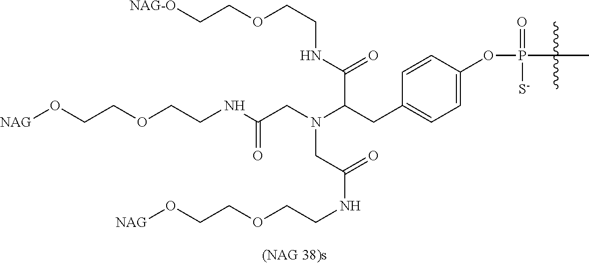

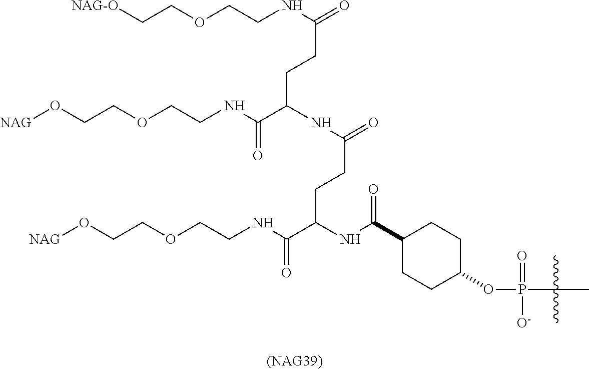

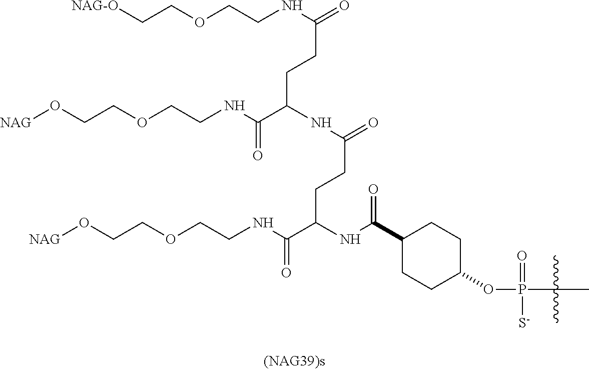

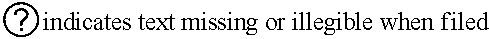

### Modified Nucleotides

In some embodiments, the first or the second RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn, invAb), non-natural base-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ internucleoside linked) nucleotides (represented herein as 3′-OMen), 2′-F-Arabino nucleotides (represented herein as NfANA or NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification may be incorporated in the first or second RNAi agent or even in a single nucleotide thereof. The RNAi agent sense strands and antisense strands may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of the first or the second RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides. As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

### Modified Internucleoside Linkages

In some embodiments, one or more nucleotides of the first or the second RNAi agent are linked by non-standard linkages or backbones (i.e., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of the first or the second RNAi agent can contain 1, 2, 3, 4, 5, or 6 phosphorothioate linkages, an antisense strand of the first or the second RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of the first or the second RNAi agent can contain 1, 2, 3, or 4 phosphorothioate linkages, an antisense strand of the first or the second RNAi agent can contain 1, 2, 3, or 4 phosphorothioate linkages, or both the sense strand and the antisense strand independently can contain 1, 2, 3, or 4 phosphorothioate linkages.

In some embodiments, the first or the second RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, the first or the second RNAi agent antisense strand contains four phosphorothioate internucleoside linkages. In some embodiments, the four phosphorothioate internucleoside linkages are between the nucleotides at positions 1-3 from the 5′ end of the sense strand and between the nucleotides at positions 19-21, 20-22, 21-23, 22-24, 23-25, or 24-26 from the 5′ end. In some embodiments, the first or the second RNAi agent contains at least two phosphorothioate internucleoside linkages in the sense strand and three or four phosphorothioate internucleoside linkages in the antisense strand.

In some embodiments, the first or the second RNAi agent contains one or more modified nucleotides and one or more modified internucleoside linkages. In some embodiments, a 2′-modified nucleoside is combined with modified internucleoside linkage.

In some embodiments, the first and the second RNAi agents disclosed herein comprise any of the modified sequences in Table 3.

TABLE 3
Exemplary modified sequences for first and second RNAi agents

| Antisense | | Sense | |
|---|---|---|---|
| SEQ | | SEQ | |
| ID | | ID | |
| NO | Unmodified sequence (5′ → 3′) | NO | Unmodified sequence (5′ → 3′) |
| 1 | asGfsasAfaAfuugagAfgAfaGfuCfcAfsc | 10 | (NAG25)sgsuggacuuCfUfCfucaauuuucus(invAb) |
| 1 | asGfsasAfaAfuugagAfgAfaGfuCfcAfsc | 11 | (NAG37)s(invAb)sguggacuuCfUfCfucaauuuucus(invAb) |
| 1 | asGfsasAfaAfuugagAfgAfaGfuCfcAfsc | 13 | (NAG25)s(invAb)sguggacuuCfUfCfucaauuuucus(invAb) |
| 2 | asGfsasAfaAfuUfgAfgAfgAfaGfuCfcasc | 10 | (NAG25)sgsuggacuuCfUfCfucaauuuucus(invAb) |
| 2 | asGfsasAfaAfuUfgAfgAfgAfaGfuCfcasc | 11 | (NAG37)s(invAb)sguggacuuCfUfCfucaauuuucus(invAb) |
| 2 | asGfsasAfaAfuUfgAfgAfgAfaGfuCfcasc | 13 | (NAG25)s(invAb)sguggacuuCfUfCfucaauuuucus(invAb) |
| 3 | asGfsasAfaAfuUfgAfgAfgAfaGfuCfcacusu | 10 | (NAG25)sgsuggacuuCfUfCfucaauuuucus(invAb) |
| 3 | asGfsasAfaAfuUfgAfgAfgAfaGfuCfcacusu | 11 | (NAG37)s(invAb)sguggacuuCfUfCfucaauuuucus(invAb) |
| 3 | asGfsasAfaAfuUfgAfgAfgAfaGfuCfcacusu | 13 | (NAG25)s(invAb)sguggacuuCfUfCfucaauuuucus(invAb) |
| 4 | asGfsasAfaAfuUfgAfgAfgAfaGfuCfcacsc | 12 | (NAG37)s(invAb)sgguggacuuCfUfCfucaauuuucus(invAb) |
| 8 | usAfscsCfaAfuUfuAfuGfcCfuAfcAfgcsg | 16 | (NAG37)s(invAb)scgcuguagGfCfAfuaaauugguas(invAb) |
| 8 | usAfscsCfaAfuUfuAfuGfcCfuAfcAfgcsg | 17 | (NAG37)csgcuguagGfCfAfuaaauugguas(invAb) |
| 8 | usAfscsCfaAfuUfuAfuGfcCfuAfcAfgcsg | 18 | (NAG25)s(invAb)scgcuguagGfCfAfuaaauugguas(invAb) |

A = adenosine-3′-phosphate;
C = cytidine-3′-phosphate;
G = guanosine-3′-phosphate;
U = uridine-3′-phosphate
n = any 2′-OMe modified nucleotide
a = 2′-O-methyladenosine-3′-phosphate
as = 2′-O-methyladenosine-3′-phosphorothioate
c = 2′-O-methylcytidine-3′-phosphate
cs = 2′-O-methylcytidine-3′-phosphorothioate
g = 2′-O-methylguanosine-3′-phosphate
gs = 2′-O-methylguanosine-3′-phosphorothioate
t = 2′-O-methyl-5-methyluridine-3′-phosphate
ts = 2′-O-methyl-5-methyluridine-3′-phosphorothioate
u = 2′-O-methyluridine-3′-phosphate
us = 2′-O-methyluridine-3′-phosphorothioate
Nf = any 2′-fluoro modified nucleotide
Af = 2′-fluoroadenosine-3′-phosphate
Afs = 2′-fluoroadenosine-3′-phosporothioate
Cf = 2′-fluorocytidine-3′-phosphate
Cfs = 2′-fluorocytidine-3′-phosphorothioate
Gf = 2′-fluoroguanosine-3′-phosphate
Gfs = 2′-fluoroguanosine-3′-phosphorothioate
Tf = 2′-fluoro-5′-methyluridine-3′-phosphate
Tfs = 2′-fluoro-5′-methyluridine-3′-phosphorothioate
Uf = 2′-fluorouridine-3′-phosphate
Ufs = 2′-fluorouridine-3′-phosphorothioate
dN = any 2′-deoxyribonucleotide
dT = 2′-deoxythymidine-3′-phosphate
N_{UNA} = 2′,3′-seco nucleotide mimics (unlocked nucleobase analogs)
N_{LNA} = locked nucleotide
Nf_{ANA} 2′-F-Arabino nucleotide
NM = 2′-methoxyethyl nucleotide
AM = 2′-methoxyethyladenosine-3′-phosphate
AMs = 2′-methoxyethyladenosine-3′-phosphorothioate
TM = 2′-methoxyethylthymidine-3′-phosphate
TMs = 2′-methoxyethylthymidine-3′-phosphorothioate
R = ribitol
(invdN) = any inverted deoxyribonucleotide (3′-3′ linked nucleotide)
(invAb) = inverted (3′-3′ linked) abasic deoxyribonucleotide
(invAb)s = inverted (3′-3′ linked) abasic deoxyribonucleotide-5′-phosphorothioate
(invn) = any inverted 2′-OMe nucleotide (3′-3′ linked nucleotide)
s = phosphorothioate linkage

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the RNAi component comprises a first and a second RNAi gents in a ratio of about 1:1, 2:1, 3:1, 4:1 or 5:1. In some embodiments, the two HBV RNAi agents are administered in a ratio of about 2:1.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

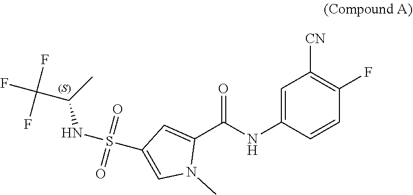

or a pharmaceutically acceptable salt thereof.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

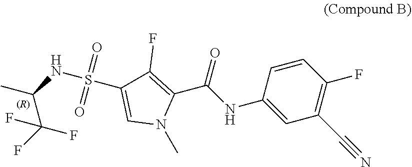

or a pharmaceutically acceptable salt thereof.

The combinations described herein can be used in any methods or kits described below.

In some embodiments, the RNAi component and the compound of Formula (I) or pharmaceutically acceptable salt thereof included in the combinations described herein are provided in separate containers. In some embodiments, the RNAi component and the compound of Formula (I) or pharmaceutically acceptable salt thereof included in the combinations described herein are provided in the same container. In some embodiments, the RNAi component includes a first RNAi agent in a first container and a second RNAi agent in a second container. For example, in an exemplary embodiment, the first RNAi agent is in a first container, the second RNAi agent is in a second container, and the compound of Formula (I) or pharmaceutically acceptable salt thereof is in a third container. In another embodiment, the RNAi component includes the first RNAi agent and the second RNAi agent in the same container. For example, in some embodiments, the first RNAi agent and the second RNAi agent are in a first container, and the compound of Formula (I) or pharmaceutically acceptable salt thereof is in a second container. Exemplary containers include vials, bags, tubes, or other suitable containers. In some embodiments, the contents of the container are sterile.

### Composition

Also provided herein is a composition comprising an RNAi component and a compound of Formula (I), wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

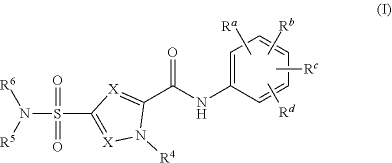

or a pharmaceutically acceptable salt thereof, wherein:
each X independently is CR⁷;
R^{a}, R^{b} and R^{c} are independently selected from the group consisting of hydrogen, fluoro, bromo, chloro, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally being substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, fluoro, chloro, bromo, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### CAM Pharmaceutical Compositions and Formulations

In some embodiments, an effective amount of the compound of Formula (I) is in the range of about 75-600 mg per dose. In some embodiments, an effective amount of the compound of Formula (I) is in the range of about 75-100 mg, about 100-150 mg, about 150-200 mg, about 200-250 mg, about 250-300 mg, about 300-400 mg, about 400-500 mg or about 500-600 mg per dose. In some embodiments, an effective amount of the compound of Formula (I) is about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, an effective amount of the compound of Formula (I) is about 100 mg, about 150 mg or about 250 mg per dose.

The compounds of the present invention, such as the compounds of Formula (I), or a pharmaceutically acceptable salt thereof, or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

### RNAi Agent Pharmaceutical Compositions and Formulations

In another aspect, described herein are methods for therapeutic and/or prophylactic treatment of diseases/disorders which are associated with HBV infection or inhibition of expression of one or more HBV genes comprising administering a pharmaceutical composition comprising one or more HBV RNAi agents that can be administered in a number of ways depending upon whether local or systemic treatment is desired. Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

In another aspect, methods described herein comprise one or more HBV RNAi agents, wherein the one or more HBV agents are prepared as pharmaceutical compositions or formulations. In some embodiments, pharmaceutical compositions include at least one HBV RNAi agent. These pharmaceutical compositions are particularly useful in the inhibition of the expression of the target mRNA in a target cell, a group of cells, a tissue, or an organism. The pharmaceutical compositions can be used to treat a subject having a disease or disorder that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering an HBV RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions including an HBV RNAi agent, thereby forming a pharmaceutical formulation suitable for in vivo delivery to a human.

The pharmaceutical compositions that include an HBV RNAi agent and methods disclosed herein may decrease the level of the target mRNA in a cell, group of cells, group of cells, tissue, or subject, including: administering to the subject a therapeutically effective amount of a herein described HBV RNAi agent, thereby inhibiting the expression of a target mRNA in the subject.

In some embodiments, the described pharmaceutical compositions including an HBV RNAi agent are used for treating or managing clinical presentations associated with HBV infection. In some embodiments, a therapeutically or prophylactically effective amount of one or more of pharmaceutical compositions is administered to a subject in need of such treatment, prevention or management. In some embodiments, administration of any of the disclosed HBV RNAi agents can be used to decrease the number, severity, and/or frequency of symptoms of a disease in a subject.

The described pharmaceutical compositions including an HBV RNAi agent can be used to treat at least one symptom in a subject having a disease or disorder that would benefit from reduction or inhibition in expression of HBV mRNA. In some embodiments, the subject is administered a therapeutically effective amount of one or more pharmaceutical compositions including an HBV RNAi agent thereby treating the symptom. In other embodiments, the subject is administered a prophylactically effective amount of one or more HBV RNAi agents, thereby preventing the at least one symptom.

The route of administration is the path by which an HBV RNAi agent is brought into contact with the body. In general, methods of administering drugs and nucleic acids for treatment of a mammal are well known in the art and can be applied to administration of the compositions described herein. The HBV RNAi agents disclosed herein can be administered via any suitable route in a preparation appropriately tailored to the particular route. Thus, herein described pharmaceutical compositions can be administered by injection, for example, intravenously, intramuscularly, intracutaneously, subcutaneously, intraarticularly, or intraperitoneally. In some embodiments, there herein described pharmaceutical compositions via subcutaneous injection.

The pharmaceutical compositions including an HBV RNAi agent described herein can be delivered to a cell, group of cells, tumor, tissue, or subject using oligonucleotide delivery technologies known in the art. In general, any suitable method recognized in the art for delivering a nucleic acid molecule (in vitro or in vivo) can be adapted for use with a herein described compositions. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the herein described pharmaceutical compositions may comprise one or more pharmaceutically acceptable excipients. In some embodiments, the pharmaceutical compositions described herein can be formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical ingredient (API, therapeutic product, e.g., HBV RNAi agent) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The HBV RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic or preventive result.

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active compound will be in the range of 25-400 mg per 1-18 weeks or 1-6 months. In some embodiments, an effective amount of an active compound will be in the range of 50-125 mg per 4 weeks or per one month. In some embodiments, an effective amount of an active ingredient will be in the range of from about 0.5 to about 3 mg/kg of body weight per dose. In some embodiments, an effective amount of an active ingredient will be in the range of from about 25-400 mg per dose. In some embodiments, an effective amount of an active ingredient will be in the range of from about 50-125 mg per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

In some embodiments, an effective amount of the RNAi component is in the range of about 25-600 mg per dose. In some embodiments, an effective amount of the RNAi component is in the range of about 25-50 mg, about 50-75 mg, about 75-100 mg, about 100-150 mg, about 150-200 mg, about 200-250 mg, about 250-300 mg, about 300-400 mg, about 400-500 mg or about 500-600 mg per dose. In some embodiments, an effective amount of the RNAi component is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, an effective amount of the RNAi component is about 25 mg, about 35 mg, about 40 mg, about 50 mg, about 100 mg or about 200 mg per dose.

The one or more (e.g., at least two) HBV RNAi agents described herein can be formulated into one single composition or separate individual compositions. In some embodiments, the HBV RNAi agents in separate individual compositions can be formulated with the same or different excipients and carriers. In some embodiments, the HBV RNAi agents in separate individual compositions agents can be administered through same or different administration routes. In some embodiments, the HBV RNAi agents are administered subcutaneously.

For treatment of disease or for formation of a medicament or composition for treatment of a disease, the pharmaceutical compositions described herein including an HBV RNAi agent can be combined with an excipient or with a second therapeutic agent or treatment including, but not limited to: a second or other RNAi agent, a small molecule drug, an antibody, an antibody fragment, and/or a vaccine.

The described HBV RNAi agents, when added to pharmaceutically acceptable excipients or adjuvants, can be packaged into kits, containers, packs, or dispensers. The pharmaceutical compositions described herein may be packaged in pre-filled syringes or vials.

In some embodiments, the composition comprises an effective amount of an RNAi component in the range of about 25-600 mg and an effective amount of a compound of Formula (I) in the range of about 75-600 mg per dose. In some embodiments, the composition comprises an effective amount of an RNAi component in the range of about 25-300 mg and an effective amount of a compound of Formula (I) in the range of about 75-300 mg per dose. In some embodiments, the composition comprises an effective amount of an RNAi of about 25 mg, about 35 mg, about 40 mg, about 50 mg, about 100 mg or about 200 mg and an effective amount of a compound of Formula (I) of about 100 mg, about 150 mg or about 250 mg per dose.

### Kit

Provided herein is a kit comprising an effective amount of an RNAi component and a compound of Formula (I) or a pharmaceutically acceptable salt thereof, wherein:

(a) the RNAi component comprises
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
(ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

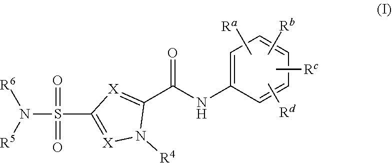

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl. The kit can comprise any combinations or compositions described herein.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In some embodiments, the kit comprises one or more doses of the compound of Formula (I) in the range of about 75-600 mg per dose. In some embodiments, the kit comprises one or more doses of the compound of Formula (I) in the range of about 75-100 mg, about 100-150 mg, about 150-200 mg, about 200-250 mg, about 250-300 mg, about 300-400 mg, about 400-500 mg or about 500-600 mg per dose. In some embodiments, the kit comprises one or more doses of the compound of Formula (I) of about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments the kit comprises one or more doses of the compound of Formula (I) of about 100 mg, about 150 mg or about 250 mg per dose.

In some embodiments, the kit comprises one or more doses of the RNAi component in the range of about 25-600 mg per dose. In some embodiments, the kit comprises one or more doses of the RNAi component in the range of about 25-50 mg, about 50-75 mg, about 75-100 mg, about 100-150 mg, about 150-200 mg, about 200-250 mg, about 250-300 mg, about 300-400 mg, about 400-500 mg or about 500-600 mg per dose. In some embodiments, the kit comprises one or more doses of the RNAi component of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg per dose. In some embodiments, the kit comprises one or more doses of the RNAi component of about 25 mg, about 35 mg, about 40 mg, about 50 mg, about 100 mg or about 200 mg per dose.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more. In some embodiments, one cycle of treatment is about 1-24 months, about 1-3 months, about 3-6 months, about 6-9 months, about 9-12 months, about 12-18 months, about 18-21 months or about 21-24 months. In some embodiments, one cycle of treatment is about 1 month, about 2 months, about 3 months, about 4 months, about 5 months, about 6 months, about 7 months, about 8 months, about 9 months, about 10 months, about 11 months, about 12 months, about 15 months, about 18 months, about 21 months or about 24 months.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### Method

Also provided herein is a method for inhibiting the expression of a Hepatitis B Virus gene in a subject in need thereof, wherein the method comprises administering to the subject an effective amount of an RNAi component and a capsid assembly modulator. Also provided herein is a method for treating a disease or disorder associated with an infection caused by Hepatitis B Virus in a subject, wherein the method comprises administering to the subject an effective amount of an RNAi component and a capsid assembly modulator. Also provided herein is a method for treating a disease or disorder associated with an infection caused by Hepatitis B Virus in a subject receiving a capsid assembly modulator therapy, wherein the method comprises administering to the subject an effective amount of an RNAi component and a capsid assembly modulator. Also provided herein is a method for inhibiting the expression of a Hepatitis B Virus gene in a subject in need thereof, wherein the subject is administered an effective amount of the compound of Formula (I) in combination with an RNAi component. Also provided herein is a method for treating a Hepatitis B Virus infection comprising contacting a cell infected with the Hepatitis B Virus infection with an effective amount of an RNAi component and a capsid assembly modulator.

In some embodiments, the RNAi component comprises: (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7, and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19.

In some embodiments, the first RNAi agent comprises SEQ ID NO: 5 and SEQ ID NO: 14. In some embodiments, the first RNAi agent comprises SEQ ID NO: 6 and SEQ ID NO: 14. In some embodiments, the first RNAi agent comprises SEQ ID NO: 7 and SEQ ID NO: 15. In some embodiments, the first RNAi agent comprises SEQ ID NO: 1 and SEQ ID NO: 10, 11 or 13. In some embodiments, the first RNAi agent comprises SEQ ID NO: 2 and SEQ ID NO: 10, 11 or 13. In some embodiments, the first RNAi agent comprises SEQ ID NO: 3 and SEQ ID NO: 10, 11, or 13. In some embodiments, the first RNAi agent comprises SEQ ID NO: 4 and SEQ ID NO: 12. In some embodiments, the second RNAi agent comprises SEQ ID NO: 9 and SEQ ID NO: 19. In some embodiments, the second RNAi agent comprises SEQ ID NO: 8 and SEQ ID NO: 16, 17 or 18.

In some embodiments, the RNAi component comprises a first RNAi agent comprising SEQ ID NO: 5 and SEQ ID NO: 14 and a second RNAi agent comprising SEQ ID NO: 9 and SEQ ID NO: 19. In some embodiments, the RNAi component comprises a first RNAi agent comprising SEQ ID NO: 6 and SEQ ID NO: 14 and a second RNAi agent comprising SEQ ID NO: 9 and SEQ ID NO: 19. In some embodiments, the RNAi component comprises a first RNAi agent comprising SEQ ID NO: 7 and SEQ ID NO: 15 and a second RNAi agent comprising SEQ ID NO: 9 and SEQ ID NO: 19.

In some embodiments, the RNAi component comprises a first RNAi agent comprising SEQ ID NO: 1 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 2 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 3 and SEQ ID NO: 10, 11 or 13 and a second RNAi agent comprising SEQ ID NO: 8 and SEQ ID NO: 16, 17 or 18. In some embodiments, the RNAi component comprises a first RNAi agent comprising SEQ ID NO: 4 and SEQ ID NO: 12 and a second RNAi agent comprising SEQ ID NO: 8 and SEQ ID NO: 16, 17 or 18.

In some embodiments, the two HBV RNAi agents are administered in a ratio of about 1:1, 2:1, 3:1, 4:1 or 5:1. In some embodiments, the two HBV RNAi agents are administered in a ratio of about 2:1.

In some embodiments, the two HBV RNAi agents are administered in a combined amount of about 25-75 mg per dose administration and in the ratio of about 2:1, about 3:1, about 1:1, about 4:1, about 5:1 or about 1:2. In some embodiments, the two HBV RNAi agents are administered in a combined amount of about 50-125 mg per dose administration and in the ratio of about 2:1, about 3:1, about 1:1, about 4:1, about 5:1 or about 1:2. In some embodiments, the two HBV RNAi agents are administered in a combined amount of about 75-150 mg per dose administration and in the ratio of about 2:1, about 3:1, about 1:1, about 4:1, about 5:1 or about 1:2. In some embodiments, the two HBV RNAi agents are administered in a combined amount of about 100-200 mg per dose administration and in the ratio of about 2:1, about 3:1, about 1:1, about 4:1, about 5:1 or about 1:2. In some embodiments, the two HBV RNAi agents are administered in a combined amount of about 150-250 mg per dose administration and in the ratio of about 2:1, about 3:1, about 1:1, about 4:1, about 5:1 or about 1:2. In some embodiments, the two HBV RNAi agents are administered in a combined amount of about 200-300 mg per dose administration and in the ratio of about 2:1, about 3:1, about 1:1, about 4:1, about 5:1 or about 1:2. In some embodiments, the two HBV RNAi agents are administered in a combined amount of about 300-400 mg per dose administration and in the ratio of about 2:1, about 3:1, about 1:1, about 4:1, about 5:1 or about 1:2. In some embodiments, the two HBV RNAi agents are administered in a combined amount of about 50-100 mg per dose administration and in the ratio of about 2:1, about 3:1, about 1:1, about 4:1, about 5:1 or about 1:2. In some embodiments, the two HBV RNAi agents are administered in a combined amount of about 25-400 mg per dose administration and in the ratio of about 2:1. In some embodiments, the two HBV RNAi agents are administered in a combined amount of about 25-75 mg per dose administration and in the ratio of about 2:1. In some embodiments, the two HBV RNAi agents are administered in a combined amount of about 50-125 mg per dose administration and in the ratio of about 2:1. In some embodiments, the two HBV RNAi agents are administered in a combined amount of about 75-150 mg per dose administration and in the ratio of about 2:1. In some embodiments, the two HBV RNAi agents are administered in a combined amount of about 100-200 mg per dose administration and in the ratio of about 2:1. In some embodiments, the two HBV RNAi agents are administered in a combined amount of about 125-225 mg per dose administration and in the ratio of about 2:1. In some embodiments, the two HBV RNAi agents are administered in a combined amount of about 150-250 mg per dose administration and in the ratio of about 2:1. In some embodiments, the two HBV RNAi agents are administered in a combined amount of about 200-300 mg per dose administration and in the ratio of about 2:1. In some embodiments, the two HBV RNAi agents are administered in a combined amount of about 300-400 mg per dose administration and in the ratio of about 2:1. In some embodiments, the two HBV RNAi agents are administered in a combined amount of about 100 mg per dose administration and in the ratio of about 2:1. In some embodiments, the two HBV RNAi agents are administered in a combined amount of about 25 mg per dose administration and in the ratio of about 2:1. In some embodiments, the two HBV RNAi agents are administered in a combined amount of about 35 mg per dose administration and in the ratio of about 2:1. In some embodiments, the two HBV RNAi agents are administered in a combined amount of about 40 mg per dose administration and in the ratio of about 2:1. In some embodiments, the two HBV RNAi agents are administered in a combined amount of about 50 mg per dose administration and in the ratio of about 2:1. In some embodiments, the two HBV RNAi agents are administered in a combined amount of about 75 mg per dose administration and in the ratio of about 2:1. In some embodiments, the two HBV RNAi agents are administered in a combined amount of about 200 mg per dose administration and in the ratio of about 2:1.

In some embodiments, the first RNAi agent is administered in an amount of about 3-650 mg per dose administration, and the second RNAi agent is administered in an amount of about 2-325 mg per dose administration. In some embodiments, the first RNAi agent is administered in an amount of about 15-150 mg per dose administration, and the second RNAi agent is administered in an amount of about 5-75 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the first RNAi agent is administered in an amount of about 15-75 mg per dose administration. In some embodiments, the second RNAi agent is administered in an amount of about 20-125 mg per dose administration. In some embodiments, the second RNAi agent is administered in an amount of about 25-50 mg per dose administration. In some embodiments, the second RNAi agent is administered in an amount of about 5-40 mg per dose administration. In some embodiments, the first RNAi agent is administered in an amount of about 17 mg per dose administration, and the second RNAi agent is administered in an amount of about 8 mg per dose administration. In some embodiments, the first RNAi agent is administered in an amount of about 23 mg per dose administration, and the second RNAi agent is administered in an amount of about 12 mg per dose administration. In some embodiments, the first RNAi agent is administered in an amount of about 27 mg per dose administration, and the second RNAi agent is administered in an amount of about 13 mg per dose administration. In some embodiments, the first RNAi agent is administered in an amount of about 33 mg per dose administration, and the second RNAi agent is administered in an amount of about 17 mg per dose administration. In some embodiments, the first RNAi agent is administered in an amount of about 67 mg per dose administration, and the second RNAi agent is administered in an amount of about 33 mg per dose administration.

In some embodiments, two RNAi agents are administered at a combined dose of 25-400 mg per dose administration. In an embodiment, two RNAi agents are administered at a combined dose of 25-400 mg, and the first RNAi agent is administered with the second RNAi agent at a ratio of 1:1. In an embodiment, the dose of each of the first and second RNAi agents is in an amount of about 12 mg for a combined dose of about 25 mg. In an embodiment, the dose of each of the first and second RNAi agents is in an amount of about 17 mg for a combined dose of about 35 mg. In an embodiment, the dose of each of the first and second RNAi agents is in an amount of about 20 mg for a combined dose of about 40 mg. In an embodiment, the dose of each of the first and second RNAi agents is in an amount of about 25 mg for a combined dose of about 50 mg. In an embodiment, the dose of each of the first and second RNAi agents is in an amount of about 50 mg for a combined dose of about 100 mg. In an embodiment, the dose of each of the first and second RNAi agents is in an amount of about 100 mg for a combined dose of about 200 mg. In an embodiment, the dose of each of the first and second RNAi agents is in an amount of about 150 mg for a combined dose of about 300 mg. In an embodiment, the dose of each of the first and second RNAi agents is in an amount of about 200 mg for a combined dose of about 400 mg.

In an embodiment, two RNAi agents are administered at a combined dose of 25-400 mg per dose, and the first RNAi agent is administered with the second RNAi agent at a ratio of 2:1. In an embodiment, the dose of the first RNAi agent is in an amount of about 16 mg, and the dose of the second RNAi agent is in an amount of about 8 mg for a combined dose of about 25 mg. In an embodiment, the dose of the first RNAi agent is in an amount of about 24 mg, and the dose of the second RNAi agent is in an amount of about 12 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the first RNAi agent is in an amount of about 65 mg, and the dose of the second RNAi agent is in an amount of about 35 mg for a combined dose of about 100 mg. In an embodiment, the dose of the first RNAi agent is in an amount of about 133 mg, and the dose of the second RNAi agent is in an amount of about 67 mg for a combined dose of about 200 mg. In an embodiment, the dose of the first RNAi agent is in an amount of about 200 mg, and the dose of the second RNAi agent is in an amount of about 100 mg for a combined dose of about 300 mg. In an embodiment, the dose of the first RNAi agent is in an amount of about 270 mg, and the dose of the second RNAi agent is in an amount of about 135 mg for a combined dose of about 400 mg.

In an embodiment, two RNAi agents are administered at a combined dose of 25-400 mg per dose, the first RNAi agent is administered with the second RNAi agent at a ratio of 3:1. In an embodiment, the dose of the first RNAi agent is in an amount of about 18 mg, and the dose of the second RNAi agent is in an amount of about 6 mg for a combined dose of about 25 mg. In an embodiment, the dose of the first RNAi agent is in an amount of about 27 mg, and the dose of the second RNAi agent is in an amount of about 9 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the first RNAi agent is in an amount of about 75 mg, and the dose of the second RNAi agent is in an amount of about 25 mg for a combined dose of about 100 mg. In an embodiment, the dose of the first RNAi agent is in an amount of about 150 mg, and the dose of the second RNAi agent is in an amount of about 50 mg for a combined dose of about 200 mg. In an embodiment, the dose of the first RNAi agent is in an amount of about 225 mg, and the dose of the second RNAi agent is in an amount of about 75 mg for a combined dose of about 300 mg. In an embodiment, the dose of the first RNAi agent is in an amount of about 300 mg, and the dose of the second RNAi agent is in an amount of about 100 mg for a combined dose of about 400 mg.

In some embodiments, the two HBV RNAi agents are administered in a combined amount of about 1-10 mg/kg per dose administration. In some embodiments, the two HBV RNAi agents are administered in a combined amount of about 1-5 mg/kg per dose administration. In some embodiments, the two HBV RNAi agents are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the first RNAi agent is administered in an amount of about 0.6-7 mg/kg per dose administration, and the second RNAi agent is administered in an amount of about 0.3-5 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the two RNAi agents are administered in about 1-18 week intervals. In some embodiments, the two RNAi agents are administered in about 1-week intervals, about 2-week intervals, about 3-week intervals, about 4-week intervals, about 5-week intervals, about 6-week intervals, about 7-week intervals, about 8-week intervals, about 9-week intervals, about 10-week intervals, about 11-week intervals, about 12-week intervals, about 13-week intervals, about 14-week intervals, about 15-week intervals, about 16-week intervals, about 17-week intervals, or about 18-week intervals. In some embodiments, the two RNAi agents are administered in about 1-6 month intervals. In some embodiments, the two RNAi agents are administered in about 1-month intervals, about 2-month intervals, about 3-month intervals, about 4-month intervals, about 5-month intervals, or about 6-month intervals. In some embodiments, the two RNAi agents are administered in about 4-week intervals or 1-month intervals. In some embodiments, the two RNAi agents are administered once per month.

In some embodiments, disclosed herein are methods for inhibiting the expression of a Hepatitis B Virus gene in a subject in need thereof comprising administering to a subject in need thereof an effective amount of a first RNAi agent comprising SEQ ID NO: 2 and SEQ ID NO: 11 and an effective amount of a second RNAi agent comprising SEQ ID NO: 16 and SEQ ID NO: 8. In some embodiments, disclosed herein are methods for treating a disease or disorder associated with an infection caused by Hepatitis B Virus in a subject comprising administering to a subject in need thereof an effective amount of a first RNAi agent comprising SEQ ID NO: 2 and SEQ ID NO: 11 and an effective amount of a second RNAi agent comprising SEQ ID NO: 16 and SEQ ID NO: 8. In some embodiments, the ratio of the first RNAi agent to the second RNAi agent administered to a subject in need thereof is about 2:1. In some embodiments, the ratio of the first RNAi agent to the second RNAi agent administered to a subject in need thereof is about 3:1. In some embodiments, the ratio of the first RNAi agent to the second RNAi agent administered to a subject in need thereof is about 1:1. In some embodiments, the ratio of the first RNAi agent to the second RNAi agent administered to a subject in need thereof is about 4:1. In some embodiments, the ratio of the first RNAi agent to the second RNAi agent administered to a subject in need thereof is about 5:1. In some embodiments, the ratio of the first RNAi agent to the second RNAi agent administered to a subject in need thereof is about 1:2.

In some embodiments, the first RNAi agent and the second RNAi agent are administered in a combined amount of about 25-400 mg per dose administration. In some embodiments, the first RNAi agent and the second RNAi agent are administered in a combined amount of about 25-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, 25-75 mg, 50-100 mg, 100-150 mg, 150-200 mg, 200-250 mg, 250-300 mg, 300-350 mg, 350-400 mg, 25-100 mg, 50-150 mg, 100-200 mg, 150-250 mg, 200-300 mg, 300-400 mg, 25-200 mg, or 200-400 mg per dose administration. In some embodiments, the first RNAi agent to the second RNAi agent are administered in a combined amount of about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg per dose administration. In some embodiments, the first RNAi agent and the second RNAi agent are administered in a combined amount of about 50 mg, about 75 mg, about 100 mg, or about 125 mg per dose administration. In some embodiments, the first RNAi agent and the second RNAi agent are administered in a combined amount of about 25 mg, about 35 mg, about 40 mg, or about 200 mg per dose administration.

In some embodiments, the first RNAi agent and the second RNAi agent are administered in about 1-18 week intervals. In some embodiments, the first RNAi agent and the second RNAi agent are administered in about 1-week intervals, about 2-week intervals, about 3-week intervals, about 4-week intervals, about 5-week intervals, about 6-week intervals, about 7-week intervals, about 8-week intervals, about 9-week intervals, about 10-week intervals, about 11-week intervals, about 12-week intervals, about 13-week intervals, about 14-week intervals, about 15-week intervals, about 16-week intervals, about 17-week intervals, or about 18-week intervals. In some embodiments, the first RNAi agent and the second RNAi agent are administered in about 1-6 month intervals. In some embodiments, the first RNAi agent and the second RNAi agent are administered in about 1-month intervals, about 2-month intervals, about 3-month intervals, about 4-month intervals, about 5-month intervals, or about 6-month intervals. In some embodiments, the first RNAi agent and the second RNAi agent are administered in about 4-week intervals or 1-month intervals.

In some embodiments, the first RNAi agent and the second RNAi agent are administered for a duration of about 1-12 months. In some embodiments, the first RNAi agent and the second RNAi agent are administered for a duration of at least about 1 month, at least about 2 months, at least about 3 months, at least about 4 months, at least about 5 months, at least about 6 months, at least about 7 months, at least about 8 months, at least about 9 months, at least about 10 months, at least about 11 months or at least about 12 months. In some embodiments, the first RNAi agent and the second RNAi agent are administered for a duration of about 1-18 weeks. In some embodiments, the first RNAi agent and the second RNAi agent are administered for a duration of at least about 1 week, at least about 5 weeks, at least about 10 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, or at least about 90 weeks. In some embodiments, capsid assembly modulator or a pharmaceutically acceptable salt is administered for a duration of about 24 weeks or 48 weeks.

In some embodiments, the first RNAi agent and the second RNAi agent are administered in a combined amount of about 25-75 mg per dose administration and in the ratio of about 2:1, about 3:1, about 1:1, about 4:1, about 5:1 or about 1:2. In some embodiments, the first RNAi agent and the second RNAi agent are administered in a combined amount of about 40-100 mg per dose administration and in the ratio of about 2:1, about 3:1, about 1:1, about 4:1, about 5:1 or about 1:2. In some embodiments, the first RNAi agent and the second RNAi agent are administered in a combined amount of about 50-125 mg per dose administration and in the ratio of about 2:1, about 3:1, about 1:1, about 4:1, about 5:1 or about 1:2. In some embodiments, the first RNAi agent and the second RNAi agent are administered in a combined amount of about 75-150 mg per dose administration and in the ratio of about 2:1, about 3:1, about 1:1, about 4:1, about 5:1 or about 1:2. In some embodiments, the first RNAi agent and the second RNAi agent are administered in a combined amount of about 100-200 mg per dose administration and in the ratio of about 2:1, about 3:1, about 1:1, about 4:1, about 5:1 or about 1:2. In some embodiments, the first RNAi agent and the second RNAi agent are administered in a combined amount of about 150-250 mg per dose administration and in the ratio of about 2:1, about 3:1, about 1:1, about 4:1, about 5:1 or about 1:2. In some embodiments, the first RNAi agent and the second RNAi agent are administered in a combined amount of about 200-300 mg per dose administration and in the ratio of about 2:1, about 3:1, about 1:1, about 4:1, about 5:1 or about 1:2. In some embodiments, the first RNAi agent and the second RNAi agent are administered in a combined amount of about 300-400 mg per dose administration and in the ratio of about 2:1, about 3:1, about 1:1, about 4:1, about 5:1 or about 1:2. In some embodiments, the first RNAi agent and the second RNAi agent are administered in a combined amount of about 50-100 mg per dose administration and in the ratio of about 2:1, about 3:1, about 1:1, about 4:1, about 5:1 or about 1:2. In some embodiments, the first RNAi agent and the second RNAi agent are administered in a combined amount of about 35-40 mg per dose administration and in the ratio of about 2:1, about 3:1, about 1:1, about 4:1, about 5:1 or about 1:2. In some embodiments, the first RNAi agent and the second RNAi agent are administered in a combined amount of about 25-400 mg per dose administration and in the ratio of about 2:1. In some embodiments, the first RNAi agent and the second RNAi agent are administered in a combined amount of about 25-75 mg per dose administration and in the ratio of about 2:1. In some embodiments, the first RNAi agent and the second RNAi agent are administered in a combined amount of about 35-40 mg per dose administration and in the ratio of about 2:1. In some embodiments, the first RNAi agent and the second RNAi agent are administered in a combined amount of about 50-125 mg per dose administration and in the ratio of about 2:1. In some embodiments, the first RNAi agent and the second RNAi agent are administered in a combined amount of about 75-150 mg per dose administration and in the ratio of about 2:1. In some embodiments, the first RNAi agent and the second RNAi agent are administered in a combined amount of about 100-200 mg per dose administration and in the ratio of about 2:1. In some embodiments, the first RNAi agent and the second RNAi agent are administered in a combined amount of about 125-225 mg per dose administration and in the ratio of about 2:1. In some embodiments, the first RNAi agent and the second RNAi agent are administered in a combined amount of about 150-250 mg per dose administration and in the ratio of about 2:1. In some embodiments, the first RNAi agent and the second RNAi agent are administered in a combined amount of about 200-300 mg per dose administration and in the ratio of about 2:1. In some embodiments, the first RNAi agent and the second RNAi agent are administered in a combined amount of about 300-400 mg per dose administration and in the ratio of about 2:1. In some embodiments, the first RNAi agent and the second RNAi agent are administered in a combined amount of about 100 mg per dose administration and in the ratio of about 2:1. In some embodiments, the first RNAi agent and the second RNAi agent are administered in a combined amount of about 25 mg per dose administration and in the ratio of about 2:1. In some embodiments, the first RNAi agent and the second RNAi agent are administered in a combined amount of about 35 mg per dose administration and in the ratio of about 2:1. In some embodiments, the first RNAi agent and the second RNAi agent are administered in a combined amount of about 40 mg per dose administration and in the ratio of about 2:1. In some embodiments, the first RNAi agent and the second RNAi agent are administered in a combined amount of about 50 mg per dose administration and in the ratio of about 2:1. In some embodiments, the first RNAi agent and the second RNAi agent are administered in a combined amount of about 200 mg per dose administration and in the ratio of about 2:1.

In some embodiments, the first RNAi agent is administered in an amount of about 3-650 mg, and the second RNAi agent is administered in an amount of about 2-325 mg per dose administration. In some embodiments, the first RNAi agent is administered in an amount of about 35-265 mg per dose administration. In some embodiments, the first RNAi agent is administered in an amount of about 50-75 mg per dose administration. In some embodiments, the second RNAi agent is administered in an amount of about 20-125 mg per dose administration. In some embodiments, the second RNAi agent is administered in an amount of about 25-50 mg per dose administration.

In some embodiments, the first RNAi agent and the second RNAi agent are administered in a combined amount of about 1-10 mg/kg per dose administration. In some embodiments, the first RNAi agent and the second RNAi agent are administered in a combined amount of about 1-5 mg/kg per dose administration. In some embodiments, the first RNAi agent and the second RNAi agent are administered in a combined amount of about 1-1.5 mg/kg, about 1.5-2.0 mg/kg, about 2.0-2.5 mg/kg, about 2.5-3.0 mg/kg, about 3.0-3.5 mg/kg, about 3.5-4.0 mg/kg, about 4.0-4.5 mg/kg, about 4.5-5.0 mg/kg, about 5.0-5.5 mg/kg, about 5.5-6.0 mg/kg, about 6.0-6.5 mg/kg, about 6.5-7.0 mg/kg, about 7.0-7.5 mg/kg, about 7.5-8.0 mg/kg, about 8.0-8.5 mg/kg, about 8.5-9.0 mg/kg, about 9.0-9.5 mg/kg, about 9.5-10 mg/kg, about 1-2.5 mg/kg, about 2.5-5.0 mg/kg, about 5.0-7.5 mg/kg, about 7.5-10 mg/kg, about 1-5.0 mg/kg, or about 5.0-10 mg/kg per dose administration.

In some embodiments, the second RNAi agent is administered in an amount of about 0.3-5 mg/kg per dose administration, and the first RNAi agent is administered in an amount of about 0.6-7 mg/kg per dose administration. In some embodiments, the second RNAi agent is administered in an amount of about 0.5-2.5 mg/kg per dose administration. In some embodiments, the second RNAi agent is administered in an amount of about 0.3-1.5 mg/kg per dose administration. In some embodiments, the first RNAi agent is administered in an amount of about 0.6-5 mg/kg per dose administration. In some embodiments, the first RNAi agent is administered in an amount of about 1-2.5 mg/kg per dose administration.

In some embodiments, the first RNAi agent and the second RNAi agent are administered at a combined dose of 25-400 mg per dose administration. In an embodiment, the first RNAi agent and the second RNAi agent are administered at a combined dose of 25-400 mg, and the first RNAi agent is administered with the second RNAi agent at a ratio of 1:1. In an embodiment, the dose of the first RNAi agent is administered with the second RNAi agent is in an amount of about 12 mg for a combined dose of about 25 mg. In an embodiment, the dose of each of the first RNAi agent and the second RNAi agent is in an amount of about 17 mg for a combined dose of about 35 mg. In an embodiment, the dose of each of the first RNAi agent and the second RNAi agent is in an amount of about 20 mg for a combined dose of about 40 mg. In an embodiment, the dose of each of the first RNAi agent and the second RNAi agent is in an amount of about 25 mg for a combined dose of about 50 mg. In an embodiment, the dose of each of the first RNAi agent and the second RNAi agent is in an amount of about 50 mg for a combined dose of about 100 mg. In an embodiment, the dose of each of the first RNAi agent and the second RNAi agent is in an amount of about 100 mg for a combined dose of about 200 mg. In an embodiment, the dose of each of the first RNAi agent and the second RNAi agent is in an amount of about 150 mg for a combined dose of about 300 mg. In an embodiment, the dose of each of the first RNAi agent and the second RNAi agent is in an amount of about 200 mg for a combined dose of about 400 mg.

In an embodiment, the first RNAi agent and the second RNAi agent are administered at a combined dose of 25-400 mg per dose, and the second RNAi agent is administered with the first RNAi agent at a ratio of 1:2. In an embodiment, the dose of the first RNAi agent is in an amount of about 16 mg, and the dose of the second RNAi agent is in an amount of about 8 mg for a combined dose of about 25 mg. In an embodiment, the dose of the second RNAi agent is in an amount of about 12 mg, and the dose of the first RNAi agent is in an amount of about 24 mg for a combined dose of about 35 mg. In an embodiment, the dose of the first RNAi agent is in an amount of about 27 mg, and the dose of the second RNAi agent is in an amount of about 13 mg for a combined dose of about 40 mg. In an embodiment, the dose of the first RNAi agent is in an amount of about 33 mg, and the dose of the second RNAi agent is in an amount of about 17 mg for a combined dose of about 50 mg. In an embodiment, the dose of the second RNAi agent is in an amount of about 35 mg, and the dose of the first RNAi agent is in an amount of about 65 mg for a combined dose of about 100 mg. In an embodiment, the dose of v is in an amount of about 67 mg, and the dose of the first RNAi agent is in an amount of about 133 mg for a combined dose of about 200 mg. In an embodiment, the dose of the second RNAi agent is in an amount of about 100 mg, and the dose of the first RNAi agent is in an amount of about 200 mg for a combined dose of about 300 mg. In an embodiment, the dose of the second RNAi agent is in an amount of about 135 mg, and the dose of the first RNAi agent is in an amount of about 270 mg for a combined dose of about 400 mg.

In an embodiment, the first RNAi agent and the second RNAi agent are administered at a combined dose of 25-400 mg per dose, the second RNAi agent is administered with the first RNAi agent at a ratio of 1:3. In an embodiment, the dose of the first RNAi agent is in an amount of about 18 mg, and the dose of the second RNAi agent is in an amount of about 6 mg for a combined dose of about 25 mg. In an embodiment, the dose of the second RNAi agent is in an amount of about 9 mg, and the dose of the first RNAi agent is in an amount of about 27 mg for a combined dose of about 35 mg. In an embodiment, the dose of the first RNAi agent is in an amount of about 30 mg, and the dose of the second RNAi agent is in an amount of about 10 mg for a combined dose of about 40 mg. In an embodiment, the dose of the first RNAi agent is in an amount of about 36 mg, and the dose of the second RNAi agent is in an amount of about 12 mg for a combined dose of about 50 mg. In an embodiment, the dose of the second RNAi agent is in an amount of about 25 mg, and the dose of the first RNAi agent is in an amount of about 75 mg for a combined dose of about 100 mg. In an embodiment, the dose of the second RNAi agent is in an amount of about 50 mg, and the dose of the first RNAi agent is in an amount of about 150 mg for a combined dose of about 200 mg. In an embodiment, the dose of the second RNAi agent is in an amount of about 75 mg, and the dose of the first RNAi agent is in an amount of about 225 mg for a combined dose of about 300 mg. In an embodiment, the dose of the second RNAi agent is in an amount of about 100 mg, and the dose of the first RNAi agent is in an amount of about 300 mg for a combined dose of about 400 mg.

In some embodiments, about 1 mg/kg (mpk) of the first RNAi agent and about 1 mg/kg of the second RNAi agent are administered to a subject in need thereof. In some embodiments, about 1.5 mg/kg of the first RNAi agent and about 1.5 mg/kg of the second RNAi agent are administered to a subject in need thereof. In some embodiments, about 2.0 mg/kg of the first RNAi agent and about 1.0 mg/kg of the second RNAi agent are administered to a subject in need thereof. In some embodiments, about 3.0 mg/kg of the first RNAi agent and about 1.0 mg/kg of the second RNAi agent are administered to a subject in need thereof. In some embodiments, about 3.2 mg/kg of the first RNAi agent and about 0.8 mg/kg of the second RNAi agent are administered to a subject in need thereof. In some embodiments, about 2.7 mg/kg of the first RNAi agent and about 1.3 mg/kg of the second RNAi agent are administered to a subject in need thereof. In some embodiments, about 4.0 mg/kg of the first RNAi agent and about 1.0 mg/kg of the second RNAi agent are administered to a subject in need thereof. In some embodiments, about 3.3 mg/kg of the first RNAi agent and about 1.7 mg/kg of the second RNAi agent are administered to a subject in need thereof. In some embodiments, between about 0.05 and about 5 mg/kg of the first RNAi agent and between about 0.05 and about 5 mg/kg of the second RNAi agent are administered to a subject in need thereof. In some embodiments, about the first RNAi agent and about the second RNAi agent are administered separately (e.g., in separate injections). In some embodiments, the respective dose of the first RNAi agent and the respective dose of the second RNAi agent are administered together (e.g., in the same injection). In some embodiments, the respective dose of the first RNAi agent and the respective dose of the second RNAi agent are prepared in a single pharmaceutical composition.

In another aspect, the capsid assembly modulator is a compound of Formula (I):

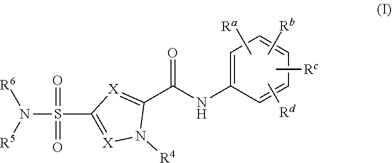

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

In some embodiments, the compound of Formula (I) is a compound of Formula (II):

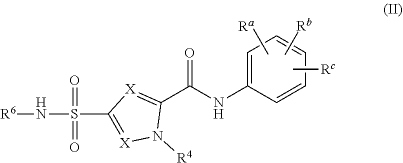

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

In some embodiments of compounds of Formula (I) or (II), R^{a}, R^{b} and R^{c} are independently hydrogen, fluoro, bromo, chloro, or CN. In some embodiments of compounds of Formula (I) or (II), R⁴ is C₁-C₃ alkyl. In some embodiments of compounds of Formula (I) or (II), R⁴ is methyl. In some embodiments of compounds of Formula (I) or (II), R⁶ is C₂-C₆ alkyl optionally substituted with one or more of —OH, fluoro, or C₁-C₄ alkyl optionally substituted with R¹⁰. In some embodiments of compounds of Formula (I) or (II), R⁶ is C₂-C₆ alkyl substituted with one or more fluoro. In some embodiments of compounds of Formula (I) or (II), each R⁷ is independent hydrogen, halogen or methyl. In some embodiments of compounds of Formula (I) or (II), at least one R⁷ is hydrogen.

In another aspect, the CAM compound is selected from the group consisting of:

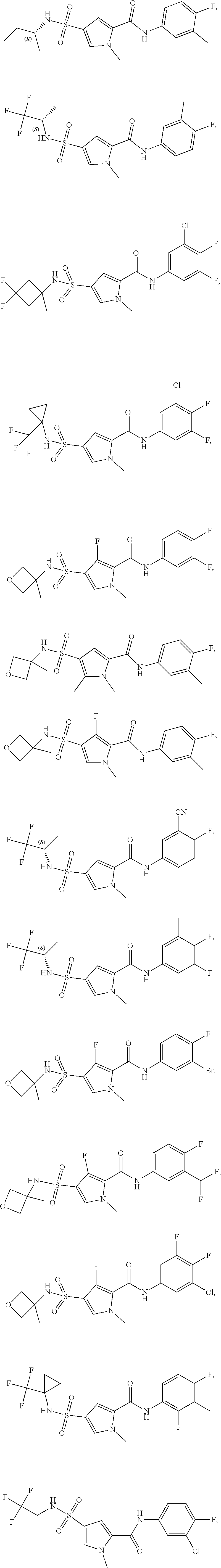

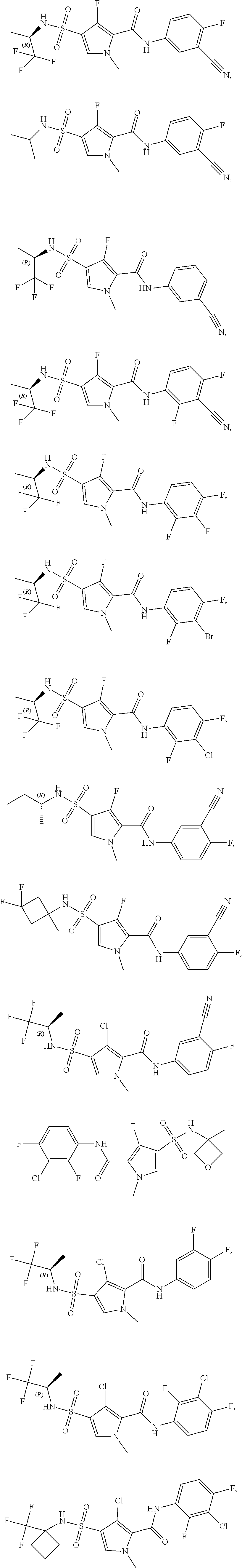

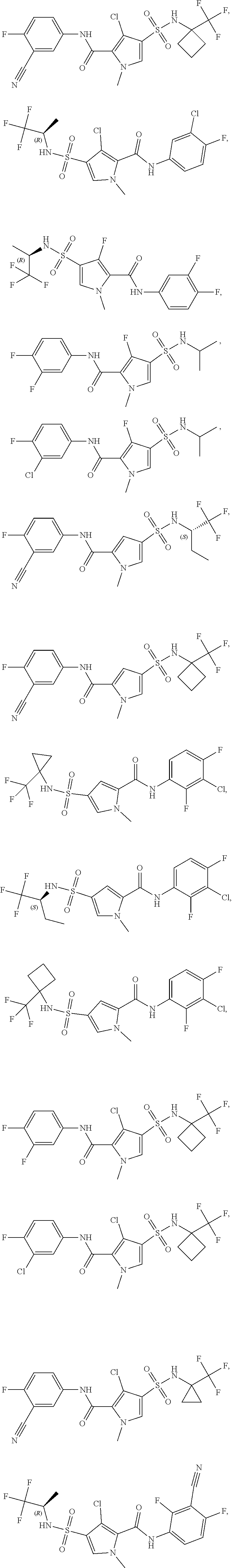

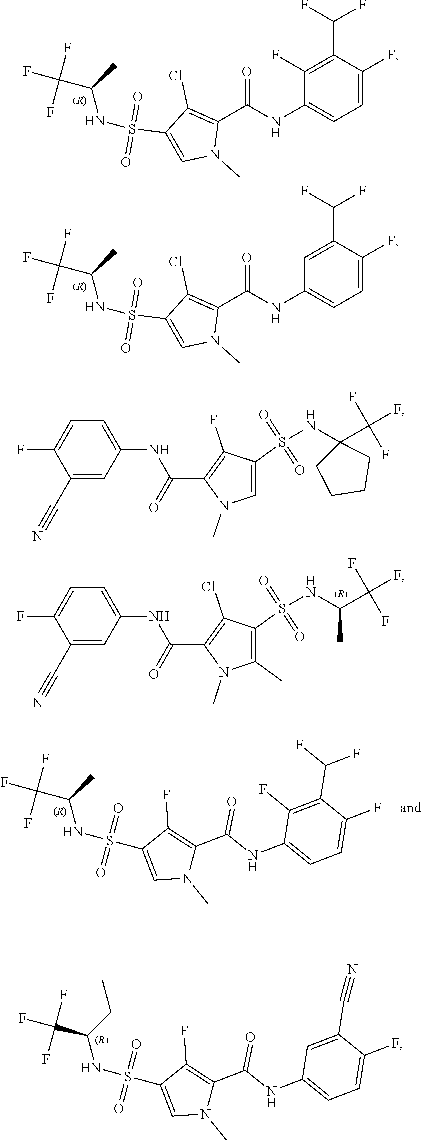

or a pharmaceutically acceptable salt of any of the foregoing.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

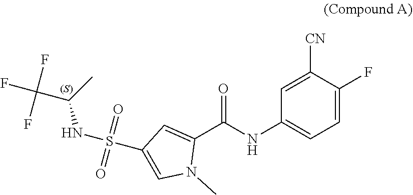

or a pharmaceutically acceptable salt thereof.

In some embodiments, the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

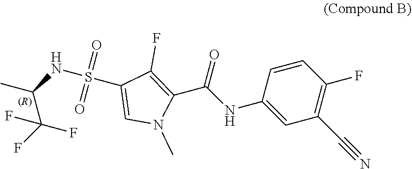

or a pharmaceutically acceptable salt thereof.

In some embodiments, the capsid assembly modulator or a pharmaceutically acceptable salt is administered in the amount of about 50-1000 mg, about 50-75 mg, about 75-100 mg, about 100-150 mg, about 150-200 mg, about 200-250 mg, about 250-300 mg, about 300-350 mg, about 350-400 mg, about 400-450 mg, about 450-500 mg, about 50-750 mg, or about 750-100 mg. In some embodiments, the capsid assembly modulator or a pharmaceutically acceptable salt is administered in the amount of about 100 mg, about 150 mg, about 200 mg, about 250 mg, or about 500 mg. In some embodiments, the capsid assembly modulator or a pharmaceutically acceptable salt is administered in the amount of about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg or about 600 mg. In some embodiments, the capsid assembly modulator or a pharmaceutically acceptable salt is administered in the amount of about 100 mg, about 150 mg or about 250 mg. In some embodiments, capsid assembly modulator or a pharmaceutically acceptable salt is administered for a duration of at least about 1 week, at least about 5 weeks, at least about 10 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 65 weeks, at least about 70 weeks, at least about 75 weeks, at least about 80 weeks, or at least about 90 weeks. In some embodiments, capsid assembly modulator or a pharmaceutically acceptable salt is administered for a duration of about 24 weeks or 48 weeks. In some embodiments, the capsid assembly modulator or a pharmaceutically acceptable salt is administered daily, every other day, every week, every 2 weeks, every 3 weeks or every month.

In some embodiments, the capsid assembly modulator or a pharmaceutically acceptable salt is formulated in a solid form, such as a tablet or capsule. In some embodiments, the capsid assembly modulator or a pharmaceutically acceptable salt is formulated in in a liquid form, such as suspensions, solutions, emulsions, or syrups, or may be lyophilized. In some embodiments, the RNAi component is formulated in a solid form, such as a tablet or capsule. In some embodiments, the RNAi component is formulated for subcutaneous injection. In some embodiments, the RNAi component is formulated in in a liquid form, such as suspensions, solutions, emulsions, or syrups, or may be lyophilized.

In some embodiments, the RNAi component and the capsid assembly modulator are administered simultaneously or intermittently. In some embodiments, the RNAi component and the capsid assembly modulator are administered are formulated separately and administered with different dosing frequencies. In some embodiments, the RNAi component and the capsid assembly modulator are formulated as one or separate compositions. In some embodiments, the RNAi component and is formulated as a solution and administered once per month via subcutaneous injection. In some embodiments, the capsid assembly modulator is formulated as an oral tablet and administered daily.

In some embodiments, the RNAi component is administered in the amount of about 50-250 mg once a month via subcutaneous injection while the compound of Formula (I) is administered in the amount of about 100-500 mg daily in the form of a tablet. In some embodiments, the RNAi component is administered in the amount of about 50 mg, about 75 mg, about 100 mg, or about 125 mg. In some embodiments, the compound of Formula (I) is administered in the amount of 150 mg, 200 mg, 250 mg or 300 mg. In some embodiments, the RNAi component is administered in the amount of about 100 mg once a month via subcutaneous injection while the compound of Formula (I) is administered in the amount of about 250 mg daily in the form of a tablet. In some embodiments, the RNAi component comprises a first RNAi agent comprising SEQ ID NO: 2 and SEQ ID NO: 11 and a second RNAi agent comprising SEQ ID NO: 16 and SEQ ID NO: 8 in a ratio of 2:1. In some embodiments, the compound of Formula (I) is compound A:

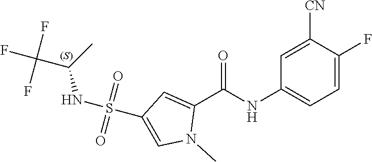

or a pharmaceutically acceptable salt thereof. In some embodiments, the compound of Formula (I) is compound B:

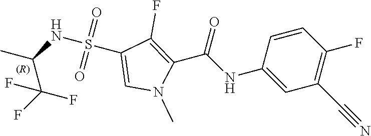

or a pharmaceutically acceptable salt thereof.

In some embodiments, the RNAi component is administered in the amount of about 25-200 mg once a month via subcutaneous injection while the compound of Formula (I) is administered in the amount of about 75-300 mg daily in the form of a tablet. In some embodiments, the RNAi component is administered in the amount of about 35 mg, about 40 mg, about 50 mg, about 100 mg, or about 200 mg. In some embodiments, the compound of Formula (I) is administered in the amount of 75 mg, 150 mg, 250 mg or 300 mg. In some embodiments, the RNAi component is administered in the amount of about 40 mg once a month via subcutaneous injection while the compound of Formula (I) is administered in the amount of about 250 mg daily in the form of a tablet. In some embodiments, the RNAi component is administered in the amount of about 100 mg once a month via subcutaneous injection while the compound of Formula (I) is administered in the amount of about 250 mg daily in the form of a tablet. In some embodiments, the RNAi component is administered in the amount of about 200 mg once a month via subcutaneous injection while the compound of Formula (I) is administered in the amount of about 250 mg daily in the form of a tablet. In some embodiments, the RNAi component comprises a first RNAi agent comprising SEQ ID NO: 2 and SEQ ID NO: 11 and a second RNAi agent comprising SEQ ID NO: 16 and SEQ ID NO: 8 in a ratio of 2:1. In some embodiments, the compound of Formula (I) is compound A:

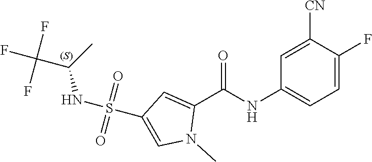

or a pharmaceutically acceptable salt thereof. In some embodiments, the compound of Formula (I) is compound B:

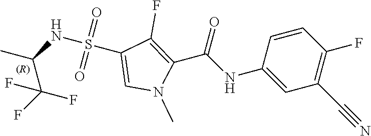

or a pharmaceutically acceptable salt thereof.

In some embodiments, the method further comprises administering a nucleoside analog. In some embodiments, the nucleoside analog is entecavir, tenofovir disoproxil fumarate or tenofovir alafenamide. In some embodiments, the nucleoside analog is lamivudine (LAM), telbivudine, or adefovir. In some embodiments, the nucleoside analog is entecavir and it is administered in the amount of about 0.01-5 mg, about 0.01-0.05 mg, about 0.05-0.1 mg, about 0.1-0.5 mg, about 0.5-1 mg, about 1-2 mg, about 2-3 mg, about 3-4 mg or about 4-5 mg. In some embodiments, the nucleoside analog is entecavir and it is administered in the amount of about 0.5 mg. In some embodiments, the nucleoside analog is tenofovir disoproxil fumarate and it is administered in the amount of about 100-500 mg, about 100-150 mg, about 150-200 mg, about 200-250 mg, about 250-300 mg, 300-400 mg, about 400-500 mg. In some embodiments, the nucleoside analog is tenofovir disoproxil fumarate and it is administered in the amount of about 300 mg In some embodiments, the nucleoside analog is tenofovir alafenamide and it is administered in the amount of about 5-100 mg, about 5-25 mg, about 25-50 mg, about 50-75 or about 75-100 mg. In some embodiments, the nucleoside analog is tenofovir alafenamide and it is administered in the amount of about 25 mg. In some embodiments, the patients have been exposed to the nucleoside analog prior to the combination therapy. In some embodiments, the patients have been administered the nucleoside analog for at least 1 month, at least 3 months, at least 6 months, or at least 1 year prior to receiving the combination therapy.

In some embodiments, the patients are screened for HBeAg status prior to administration of the combination therapy. In some embodiments, the patients are HBeAg positive. In some embodiments, the patients are HBeAg negative.

In some embodiments, the HBsAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBeAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBcrAg level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1. In some embodiments, the HBV DNA level in the patient is reduced by at least about log₁₀ 0.5, about log₁₀ 1, about log₁₀ 1.5, about log₁₀ 2, about log₁₀ 3, about log₁₀ 4, about log₁₀ 5 or about log₁₀ 7.5 from base line on Day 1. In some embodiments, the HBV RNA level in the patient is reduced by at least about log₁₀ 0.5, about log₁₀ 0.75, about log₁₀ 1, about log₁₀ 1.25, about log₁₀ 1.5, about log₁₀ 1.75, about log₁₀ 2 or about log₁₀ 2.5 from base line on Day 1.

In some embodiments, the patients have been receiving a capsid assembly modulator for at least about 1 month, about 3 month, about 6 months or about 1 year before the administration of the RNAi component.

### Exemplary Embodiments

The following embodiments are exemplary and should not be considered as limiting the invention as described herein.

### Embodiment 1

A method for inhibiting the expression of a Hepatitis B Virus gene in a subject in need thereof, wherein the method comprises administering to the subject an effective amount of an RNAi component and a compound of Formula (I), wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

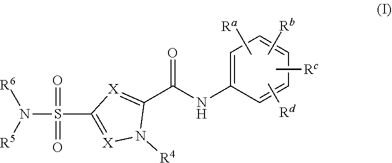

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 2

A method for treating a disease or disorder associated with an infection caused by Hepatitis B Virus in a subject, wherein the method comprises administering to the subject an effective amount of an RNAi component and a compound of Formula (I), wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

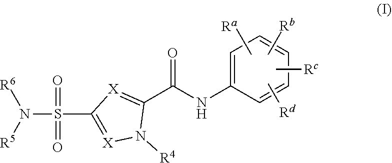

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R′)₂;
R¹⁰ is —CN, —OH, fluoro, —CHF₂, —CH₂F or —CF₃; and
R¹¹ is hydrogen or C₁-C₃ alkyl.

### Embodiment 3

A method of treating a disease or disorder associated with an infection caused by Hepatitis B Virus in a subject receiving a capsid assembly modulator therapy, wherein the method comprises administering to the subject an effective amount of an RNAi component, wherein the RNAi component comprises
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
(ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19.

### Embodiment 4

The method of Embodiment 3, wherein the capsid assembly modulator is a compound of Formula (I):

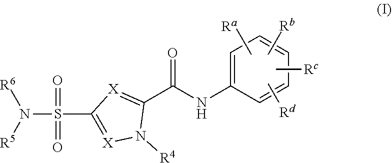

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 5

A method of treating a Hepatitis B Virus infection comprising contacting a cell infected with the Hepatitis B Virus infection with an effective amount of an RNAi component and a compound of Formula (I), or a therapeutically effective metabolite of the foregoing, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

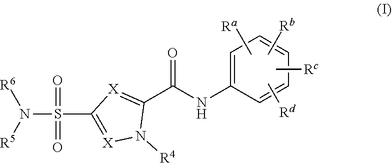

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 6

The method of any one of Embodiments 1-5, wherein the first or the second RNAi agent comprises at least one modified nucleotide or at least one modified internucleoside linkage.

### Embodiment 7

The method of any one of Embodiments 1-6, wherein substantially all of the nucleotides in the first and the second RNAi agents are modified nucleotides.

### Embodiments 8

The method of any one of Embodiments 1-7, wherein the first or the second RNAi agent further comprises a targeting ligand that is conjugated to the first or the second RNAi agent.

### Embodiment 9

The method of Embodiment 8, wherein the targeting ligand comprises N-acetyl-galactosamine.

### Embodiment 10

The method of Embodiment 9, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

### Embodiment 11

The method of Embodiment 10, wherein the targeting ligand is (NAG25), (NAG25)s, (NAG31), (NAG31)s, (NAG37), or (NAG37)s.

### Embodiment 12

The method of any one of Embodiments 8-11, wherein the targeting ligand is conjugated to the sense strand of the first or the second RNAi agent.

### Embodiment 13

The method of Embodiment 12, wherein the targeting ligand is conjugated to the 5′ terminus of the sense stand of the first or the second RNAi agent.

### Embodiment 14

The method of any one of Embodiments 1-13, wherein the first and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO:16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO:18.

### Embodiment 15

The method of any one of Embodiments 1-14, wherein the first and the second RNAi agents are each independently conjugated to a targeting ligand comprising N-acetyl-galactosamine, and the first and the second RNAi agents independently comprise a duplex selected from the group consisting of
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

### Embodiment 16

The method of any one of Embodiments 1-15, wherein the ratio of the first RNAi agent to the second RNAi agent by weight is in the range of about 1:2 to about 5:1.

### Embodiment 17

The method of Embodiment 16, wherein the ratio of the first RNAi agent to the second RNAi agent by weight is about 2:1.

### Embodiment 18

The method of any one of Embodiments 1, 2 and 4-17, wherein the compound of Formula (I) is a compound of Formula (II):

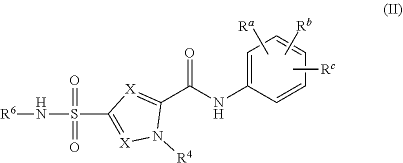

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹ is —CN, —OH, fluoro, —CHF₂, —CH₂F or —CF₃.

### Embodiment 19

The method of Embodiment 18, wherein R^{a}, R^{b} and R^{c} are independently hydrogen, fluoro, bromo, chloro, or CN.

### Embodiment 20

The method of Embodiment 18 or 19, wherein R is C₁-C₃ alkyl.

### Embodiment 21

The method of Embodiment 20, wherein R⁴ is methyl.

### Embodiment 22

The method of any one of Embodiments 18-21, wherein R⁶ is C₂-C₆ alkyl optionally substituted with one or more of —OH, fluoro, or C₁-C₄ alkyl optionally substituted with R¹⁰.

### Embodiment 23

The method of Embodiment 22, wherein R⁶ is C₂-C₆ alkyl substituted with one or more fluoro.

### Embodiment 24

The method of any one of Embodiments 18-23, wherein each R⁷ is independent hydrogen, halogen or methyl.

### Embodiment 25

The method of Embodiment 24, wherein at least one R⁷ is hydrogen.

### Embodiment 26

The method of any one of Embodiments 1-17, wherein the compound of Formula (I) is selected from the group consisting of:

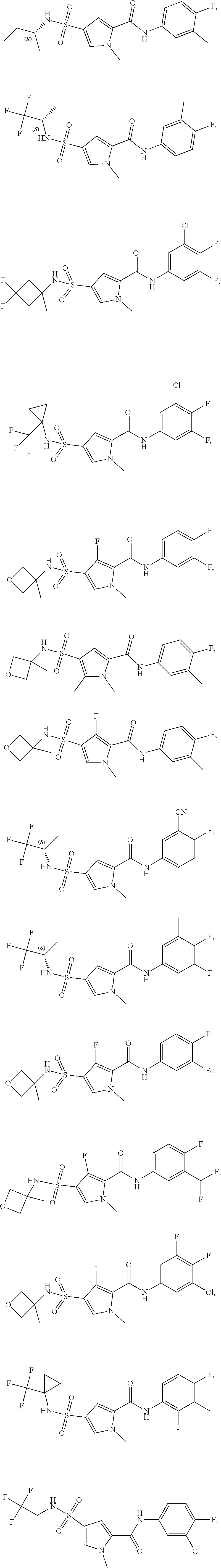

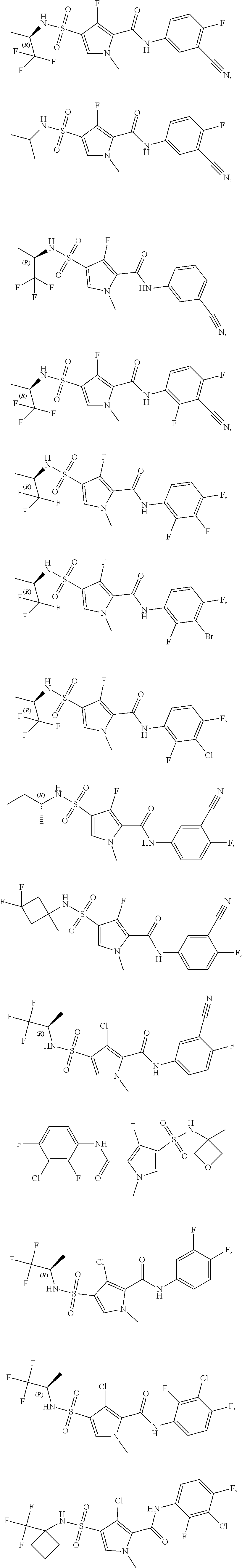

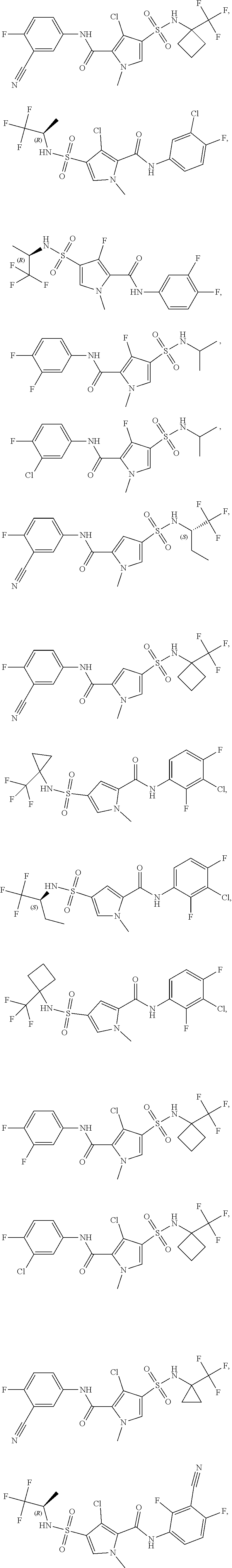

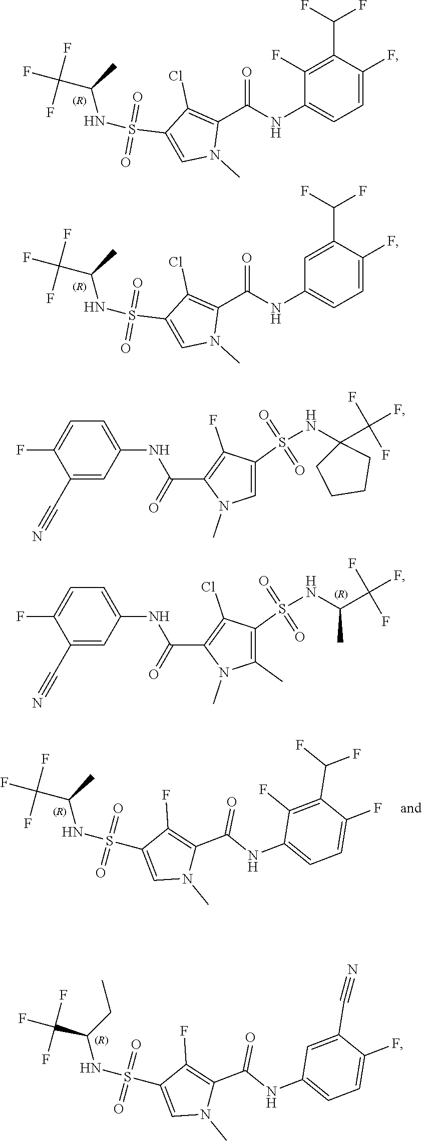

or a pharmaceutically acceptable salt of any of the foregoing.

### Embodiment 27

The method of any one of Embodiments 1-26, wherein the compound of Formula (I) is

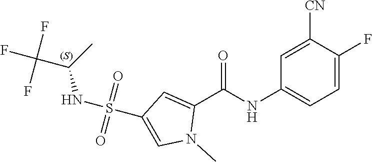

or a pharmaceutically acceptable salt thereof.

### Embodiment 28

The method of any one of Embodiments 1-26, wherein the compound of Formula (I) is

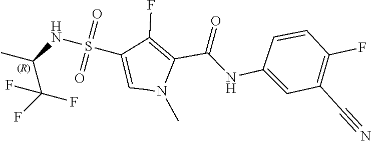

or a pharmaceutically acceptable salt thereof.

### Embodiment 29

The method of any one of Embodiments 1-5, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

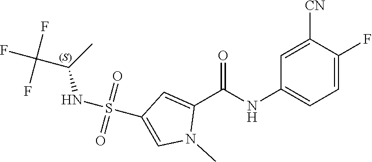

or a pharmaceutically acceptable salt thereof.

### Embodiment 30

The method of any one of Embodiments 1-5, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

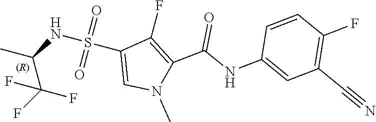

or a pharmaceutically acceptable salt thereof.

### Embodiment 31

The method of any one of Embodiments 1-30, wherein the RNAi component is administered to the subject once monthly in a dose of about 50-200 mg.

### Embodiment 32

The method of any one of Embodiments 1, 2 and 4-31, wherein the compound of Formula (I) or a pharmaceutically acceptable salt thereof is administered to the subject in a daily dose of about 100-500 mg.

### Embodiment 33

The method of any one of Embodiments 1-32, wherein the RNAi component is administered to the subject via intravenous or subcutaneous injection.

### Embodiment 34

The method of any one of Embodiments 1, 2 and 4-33, wherein the compound of Formula (I) or a pharmaceutically acceptable salt thereof is administered to the subject orally.

### Embodiment 35

The method of Embodiment 3, wherein the RNAi component is administered simultaneously or sequentially with the capsid assembly modulator.

### Embodiment 36

The method of Embodiment 3, wherein the RNAi component is administered separately from the capsid assembly modulator.

### Embodiment 37

The method of Embodiment 3, wherein the subject has been receiving the capsid assembly modulator therapy for at least about 1 month.

### Embodiment 38

The method of any one of Embodiments 1-37 further comprising administering to the subject a nucleoside analog.

### Embodiment 39

The method of Embodiment 38, wherein the nucleoside analog is entecavir, tenofovir disoproxil fumarate or tenofovir alafenamide.

### Embodiment 40

The method of Embodiment 39, wherein entecavir is administered to the subject in a daily dose of about 0.1-5 mg.

### Embodiment 41

The method of Embodiment 39, wherein tenofovir is administered to the subject in a daily dose of about 5-50 mg of tenofovir alafenamide or about 200-500 mg of tenofovir disoproxil fumarate.

### Embodiment 42

A kit comprising an effective amount of an RNAi component and a compound of Formula (I) or a pharmaceutically acceptable salt thereof, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

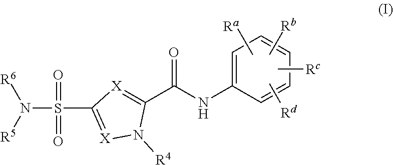

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 43

The kit of Embodiment 42 further comprising instructions for carrying out any one of the methods of Embodiments 1-41.

### Embodiment 44

The kit of Embodiment 42 or 43, wherein the compound of Formula (I) is a compound of Formula (II):

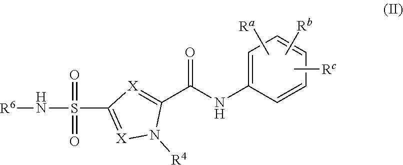

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

### Embodiment 45

The kit of any one of Embodiments 42-44 further comprising a pharmaceutically acceptable carrier, diluent, excipient or a combination of any of the foregoing.

### Embodiment 46

The kit of any one of Embodiments 42-45, wherein the first or the second RNAi agent comprises at least one modified nucleotide or at least one modified internucleoside linkage.

### Embodiment 47

The kit of any one of Embodiments 42-46, wherein substantially all of the nucleotides in the first and the second RNAi agents are modified nucleotides.

### Embodiment 48

The kit of any one of Embodiments 42-47, wherein the first or the second RNAi agent further comprises a targeting ligand that is conjugated to the first or the second RNAi agent.

### Embodiment 49

The kit of Embodiment 48, wherein the targeting ligand comprises N-acetyl-galactosamine.

### Embodiment 50

The kit of Embodiment 49, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

### Embodiment 51

The kit of Embodiment 50, wherein the targeting ligand is (NAG25), (NAG25)s, (NAG31), (NAG31)s, (NAG37), or (NAG37)s.

### Embodiment 52

The kit of anyone of Embodiments 48-51, wherein the targeting ligand is conjugated to the sense strand of the first or the second RNAi agent.

### Embodiment 53

The kit of Embodiment 52, wherein the targeting ligand is conjugated to the 5′ terminus of the sense stand of the first or the second RNAi agent.

### Embodiment 54

The kit of any one of Embodiments 42-53, wherein the first and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO:16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO:18.

### Embodiment 55

The kit of any one of Embodiments 42-54, wherein the first and the second RNAi agents are each independently conjugated to a targeting ligand comprising N-acetyl-galactosamine, and the first and the second RNAi agents independently comprise a duplex selected from the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

### Embodiment 56

The kit of any one of Embodiments 42-55, wherein the ratio of the first RNAi agent to the second RNAi agent by weight is in the range of about 1:2 to about 5:1.

### Embodiment 57

The kit of Embodiment 56, wherein the ratio of the first RNAi agent to the second RNAi agent by weight is about 2:1.

### Embodiment 58

The kit of any one of Embodiments 42-57, wherein R^{a}, R^{b} and R^{c} are independently hydrogen, fluoro, bromo, chloro, or CN.

### Embodiment 59

The kit of any one of Embodiments 42-58, wherein R⁴ is C₁-C₃ alkyl.

### Embodiment 60

The kit of Embodiment 59, wherein R⁴ is methyl.

### Embodiment 61

The kit of any one of Embodiments 42-60, wherein R⁶ is C₂-C₆ alkyl optionally substituted with one or more of —OH, fluoro, or C₁-C₄ alkyl optionally substituted with R¹⁰.

### Embodiment 62

The kit of Embodiment 61, wherein R⁶ is C₂-C₆ alkyl substituted with one or more fluoro.

### Embodiment 63

The kit of any one of Embodiments 42-62, wherein each R⁷ is independent hydrogen, halogen or methyl.

### Embodiment 64

The kit of Embodiment 63, wherein at least one R⁷ is hydrogen.

### Embodiment 65

The kit of any one of Embodiments 42-44, wherein the compound of Formula (I) is selected from the group consisting of:

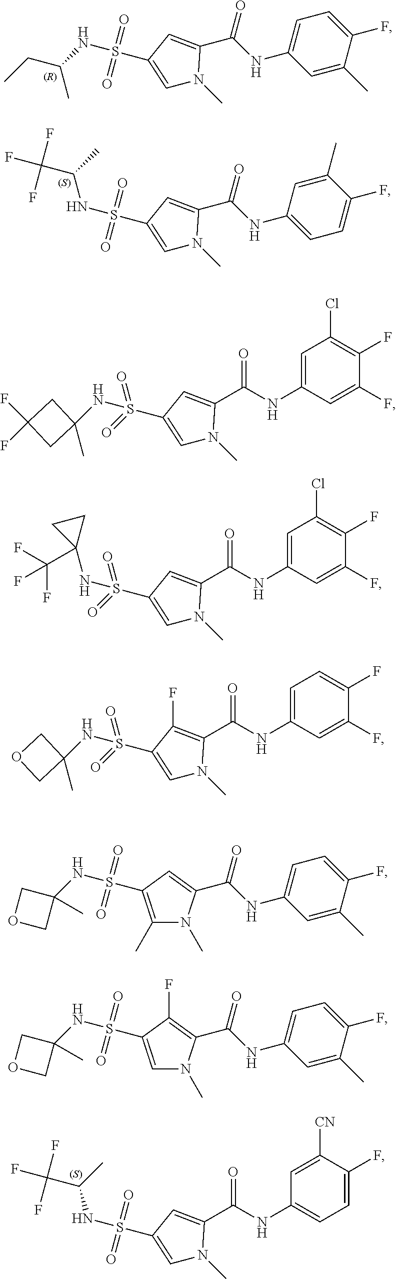

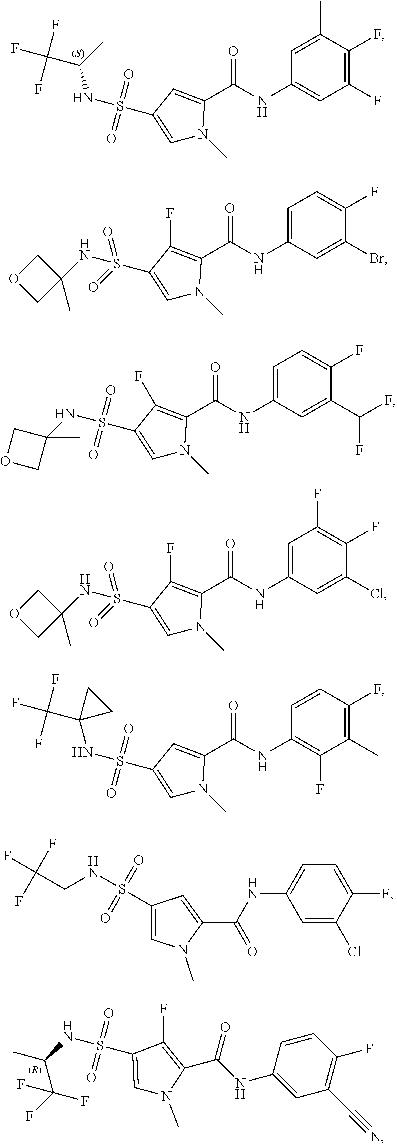

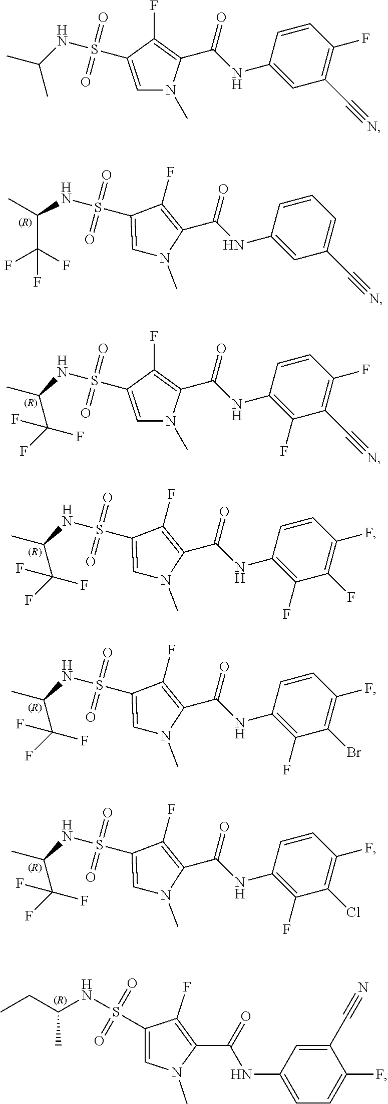

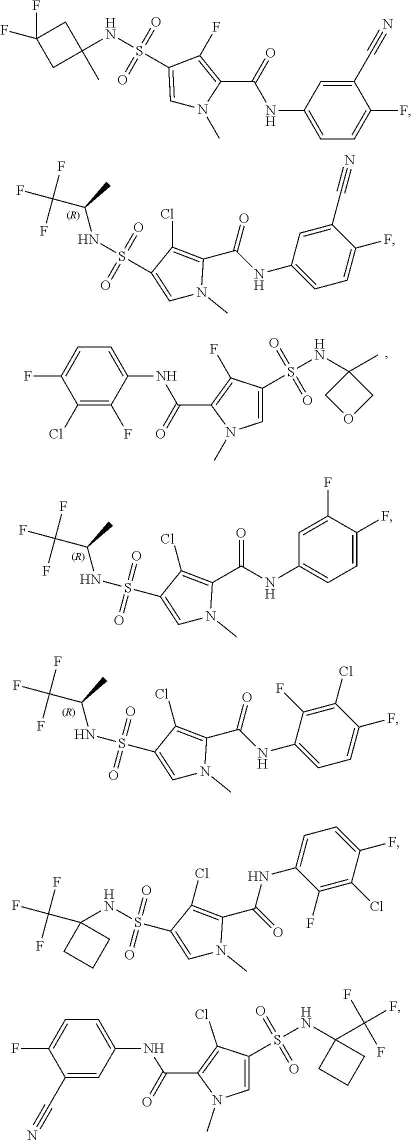

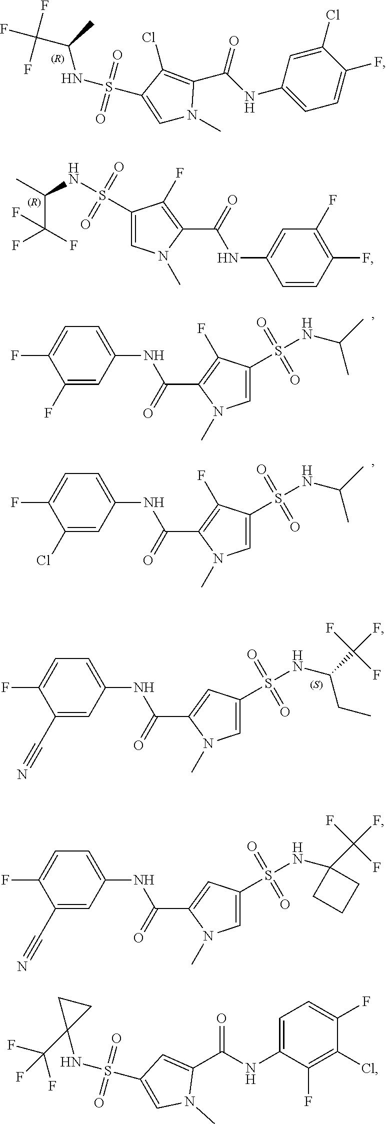

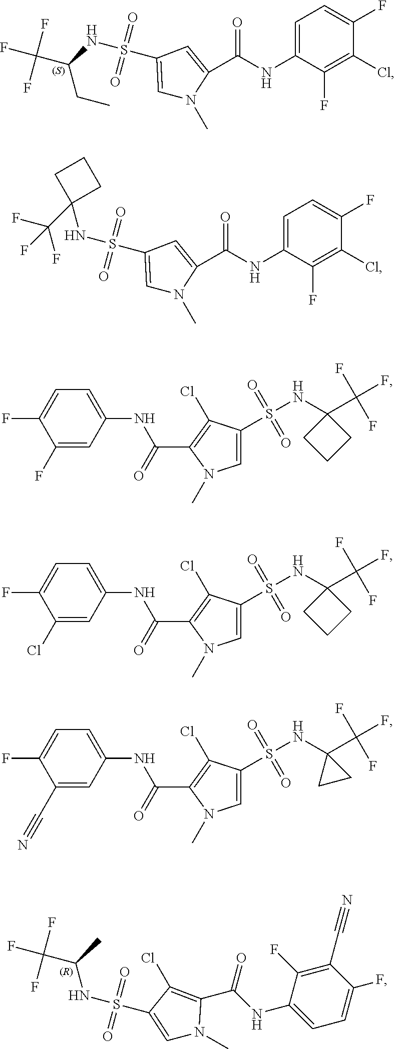

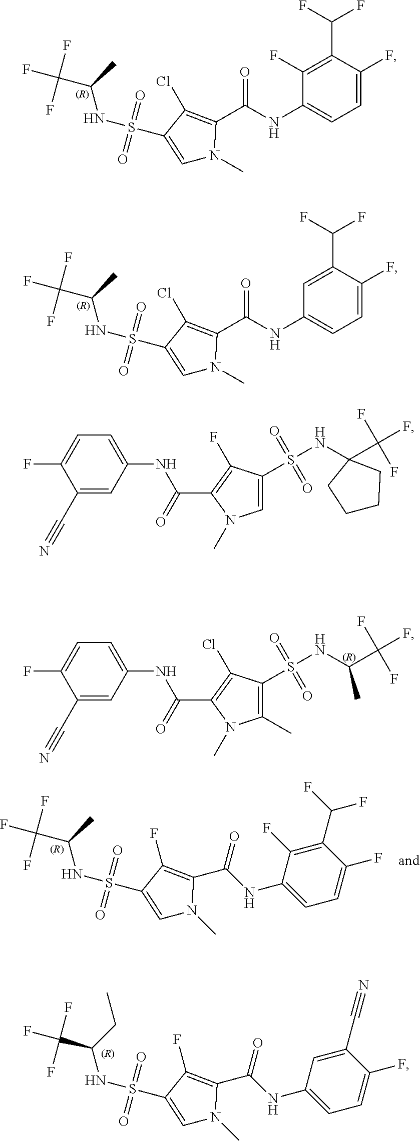

or a pharmaceutically acceptable salt of any of the foregoing.

### Embodiment 66

The kit of any one of Embodiments 42-65, wherein the compound of Formula (I) is selected from the group consisting of

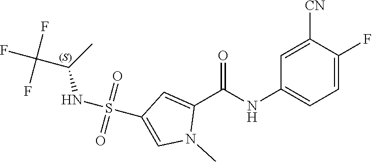

or a pharmaceutically acceptable salt thereof.

### Embodiment 67

The kit of any one of Embodiments 42-65, wherein the compound of Formula (I) is selected from the group consisting of

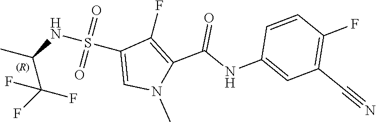

or a pharmaceutically acceptable salt thereof.

### Embodiment 68

The kit of any one of Embodiments 42-44, wherein the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

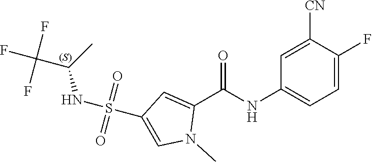

or a pharmaceutically acceptable salt thereof.

### Embodiment 69

The kit of any one of Embodiments 42-44, wherein the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

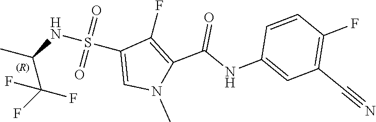

or a pharmaceutically acceptable salt thereof.

### Embodiment 70

The kit of any one of Embodiments 42-69, wherein the RNAi component and the compound of Formula (I) or a pharmaceutically acceptable salt thereof are formulated in two separation compositions.

### Embodiment 71

The kit of Embodiment 70, wherein the RNAi component is formulated for intravenous or subcutaneous injection to a subject.

### Embodiment 72

The kit of Embodiment 70 or 71, wherein the compound of Formula (I) or a pharmaceutically acceptable salt thereof is formulated for oral administration to a subject.

### Embodiment 73

A composition comprising an RNAi component and a compound of Formula (I), wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

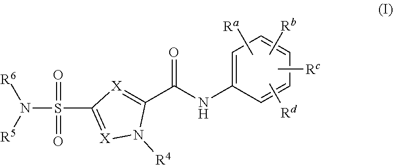

or a pharmaceutically acceptable salt thereof, wherein:
each X independently is CR⁷;
R^{a}, R^{b} and R^{c} are independently selected from the group consisting of hydrogen, fluoro, bromo, chloro, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally being substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, fluoro, chloro, bromo, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 74

The composition of Embodiment 73, wherein the compound of Formula (I) is a compound of Formula (II):

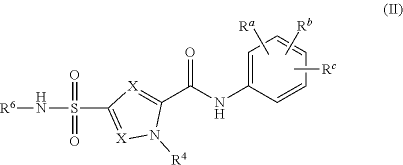

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

### Embodiment 75

The composition of Embodiment 73 or 74, wherein substantially all of the nucleotides in the first and the second RNAi agents are modified nucleotides.

### Embodiment 76

The composition of any one of Embodiments 73-75, wherein the first or the second RNAi agent further comprises a targeting ligand that is conjugated to the first or the second RNAi agent.

### Embodiment 77

The composition of Embodiment 76, wherein the targeting ligand comprises N-acetyl-galactosamine.

### Embodiment 78

The composition of Embodiment 77, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

### Embodiment 79

The composition of Embodiment 78, wherein the targeting ligand is (NAG25), (NAG25)s, (NAG31), (NAG31)s, (NAG37), or (NAG37)s.

### Embodiment 80

The composition of any one of Embodiments 76-79, wherein the targeting ligand is conjugated to the sense strand of the first or the second RNAi agent.

### Embodiment 81

The composition of Embodiment 80, wherein the targeting ligand is conjugated to the 5′ terminus of the sense stand of the first or the second RNAi agent.

### Embodiment 82

The composition of anyone of Embodiments 73-81, wherein the first and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO:16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO:18.

### Embodiment 83

The composition of any one of Embodiments 76-82, wherein the first and the second RNAi agents are each independently conjugated to a targeting ligand comprising N-acetyl-galactosamine, wherein the first and the second RNAi agents independently comprise a duplex selected form the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

### Embodiment 84

The composition of any one of Embodiments 73-83, wherein the ratio of the first RNAi agent to the second RNAi agent by weight is in the range of about 1:2 to about 5:1.

### Embodiment 85

The composition of Embodiment 84, wherein the ratio of the first RNAi agent to the second RNAi agent by weight is about 2:1.

### Embodiment 86

The composition of any one of Embodiments 73-85, wherein R^{a}, R^{b} and R^{c} are independently hydrogen, fluoro, bromo, chloro, or CN.

### Embodiment 87

The composition of any one of Embodiments 73-86, wherein R⁴ is C₁-C₃ alkyl.

### Embodiment 88

The composition of Embodiment 87, wherein R⁴ is methyl.

### Embodiment 89

The composition of any one of Embodiments 73-88, wherein R⁶ is C₂-C₆ alkyl optionally substituted with one or more of —OH, fluoro, or C₁-C₄ alkyl optionally substituted with R¹⁰.

### Embodiment 90

The composition of Embodiment 89, wherein R⁶ is C₂-C₆ alkyl substituted with one or more fluoro.

### Embodiment 91

The composition of any one of Embodiments 73-90, wherein each R⁷ is independent hydrogen, halogen or methyl.

### Embodiment 92

The composition of Embodiment 91, wherein at least one R⁷ is hydrogen.

### Embodiment 93

The composition of Embodiment 73 or 74, wherein the compound of Formula (I) is selected from the group consisting of:

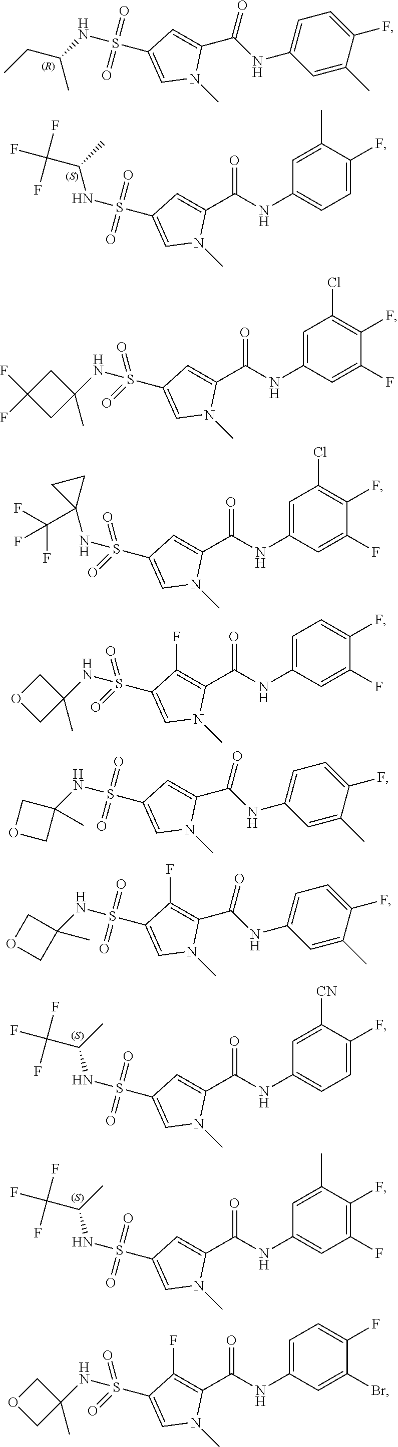

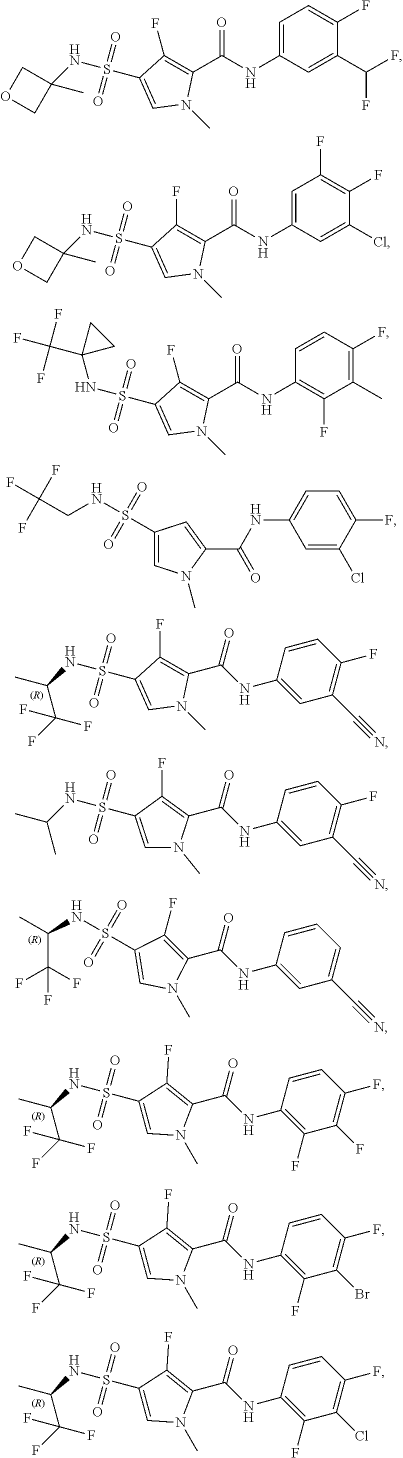

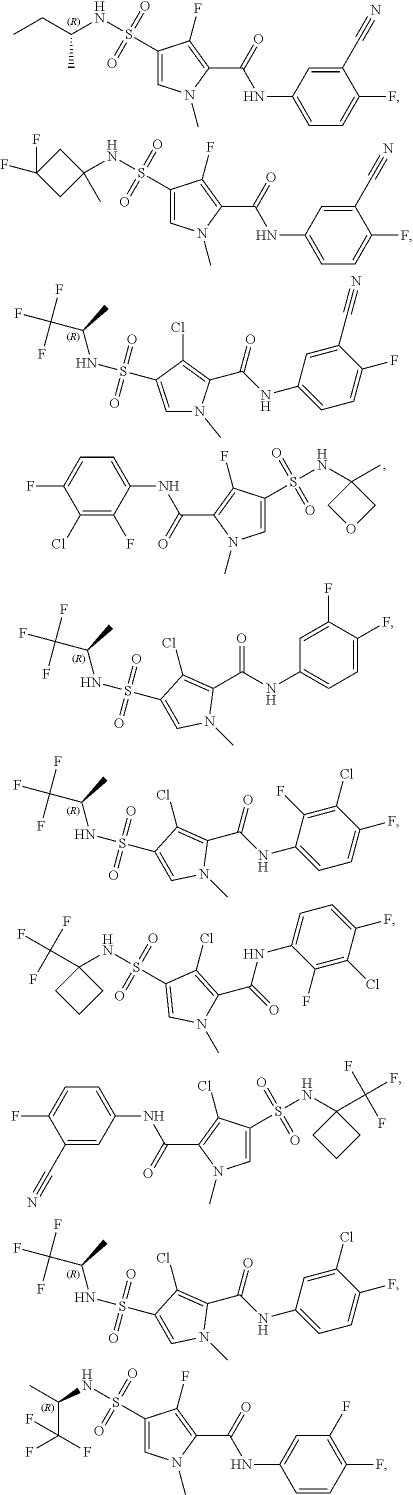

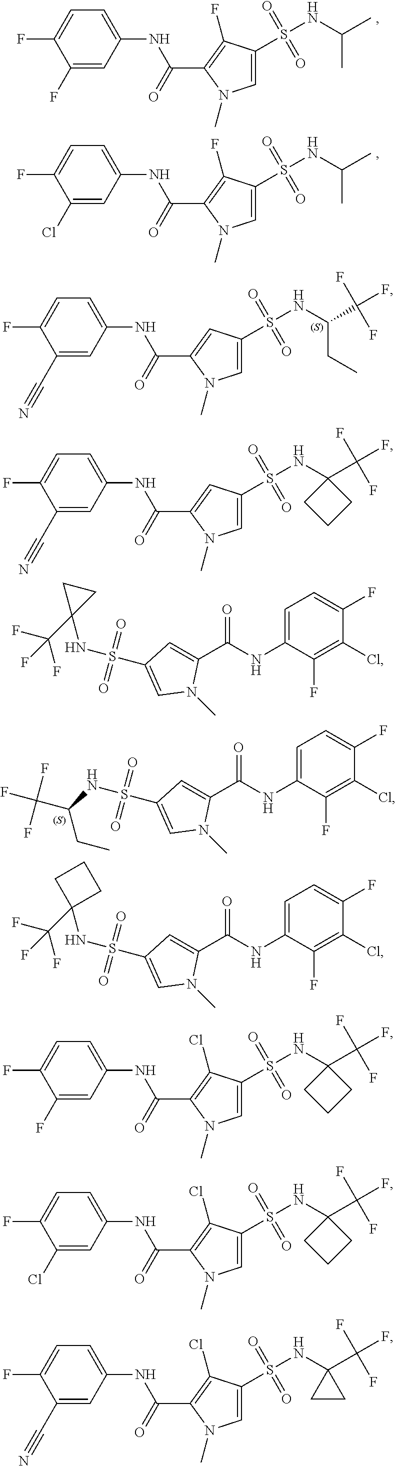

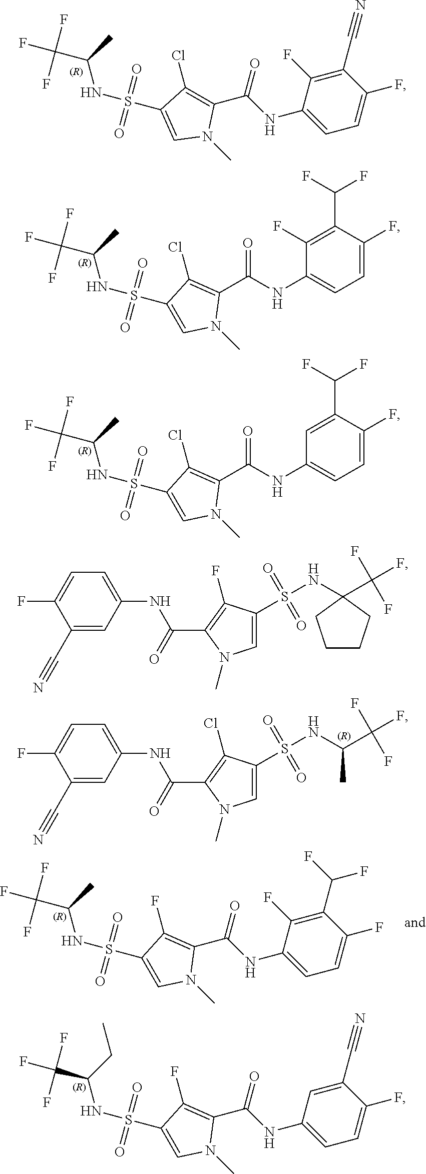

or a pharmaceutically acceptable salt of any of the foregoing.

### Embodiment 94

The composition of any one of Embodiments 73-93, wherein the compound of Formula (I) is selected from the group consisting of

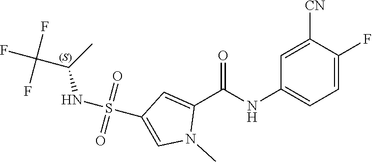

or a pharmaceutically acceptable salt thereof.

### Embodiment 95

The composition of any one of Embodiments 73-93, wherein the compound of Formula (I) is selected from the group consisting of

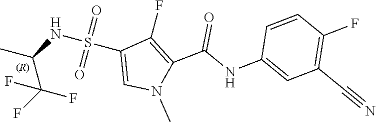

or a pharmaceutically acceptable salt thereof.

### Embodiment 96

The composition of Embodiment 73 or 74, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

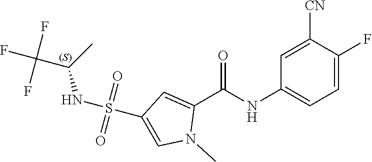

or a pharmaceutically acceptable salt thereof.

### Embodiment 97

The composition of Embodiment 73 or 74, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

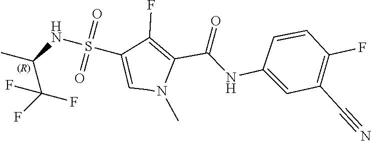

or a pharmaceutically acceptable salt thereof.

### Embodiment 98

A pharmaceutical composition comprising an effective amount of the composition of any one of Embodiments 73-97 and a pharmaceutically acceptable carrier, diluent, excipient, or a combination of any of the foregoing.

### Embodiment 99

An article of manufacture comprising a container enclosing the composition of any one of Embodiments 73-97 or the pharmaceutical composition of Embodiment 98.

### Embodiment 100

An effective amount of an RNAi component and a compound of Formula (I) in the manufacture of a medicament for treating a viral infection in a subject caused by Hepatitis B Virus, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

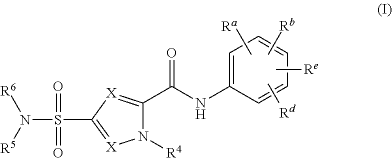

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 101

An effective amount of an RNAi component and a compound of Formula (I) in the manufacture of a medicament for inhibiting the expression of a Hepatitis B Virus gene in a subject in need thereof, wherein:
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

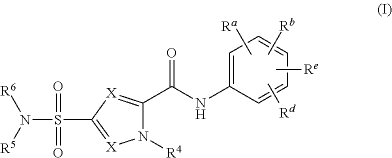

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

### Embodiment 102

A compound of Formula (I) for use in inhibiting the expression of a Hepatitis B Virus gene in a subject in need thereof, wherein the subject is administered an effective amount of the compound of Formula (I) in combination with an RNAi component, wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

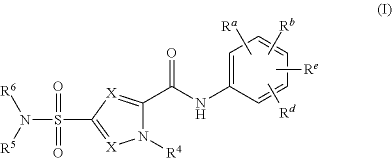

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R′)₂;
R¹⁰ is —CN, —OH, fluoro, —CHF₂, —CH₂F or —CF₃; and
R¹¹ is hydrogen or C₁-C₃ alkyl.

### Embodiment 103

A combination comprising an RNAi component and a compound of Formula (I), wherein
(a) the RNAi component comprises
   (i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7 and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
   (ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is

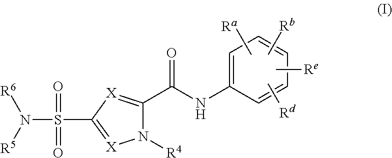

or a pharmaceutically acceptable salt thereof, wherein:
each X independently is CR⁷;
R^{a}, R^{b} and R^{c} are independently selected from the group consisting of hydrogen, fluoro, bromo, chloro, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally being substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, fluoro, chloro, bromo, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally being substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R′)₂;
R¹⁰ is —CN, —OH, fluoro, —CHF₂, —CH₂F or —CF₃; and
R¹¹ is hydrogen or C₁-C₃ alkyl.

### Embodiment 104

The combination of Embodiment 103, wherein the compound of Formula (I) is a compound of Formula (II):

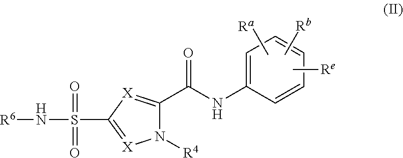

or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰;
R⁷ is hydrogen, halogen or C₁-C₃ alkyl; and
R¹⁰ is —CN, —OH, fluoro, —CHF₂, —CH₂F or —CF₃.

### Embodiment 105

The combination of Embodiment 103 or Embodiment 104, wherein substantially all of the nucleotides in the first and the second RNAi agents are modified nucleotides.

### Embodiment 106

The combination of any one of Embodiments 103-105, wherein the first or the second RNAi agent further comprises a targeting ligand that is conjugated to the first or the second RNAi agent.

### Embodiment 107

The combination of Embodiment 106, wherein the targeting ligand comprises N-acetyl-galactosamine.

### Embodiment 108

The combination of Embodiment 107, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

### Embodiment 109

The combination of Embodiment 108, wherein the targeting ligand is (NAG25), (NAG25)s, (NAG31), (NAG31)s, (NAG37), or (NAG37)s.

### Embodiment 110

The combination of any one of Embodiments 106-109, wherein the targeting ligand is conjugated to the sense strand of the first or the second RNAi agent.

### Embodiment 111

The combination of Embodiment 110, wherein the targeting ligand is conjugated to the 5′ terminus of the sense stand of the first or the second RNAi agent.

### Embodiment 112

The combination of any one of Embodiments 103-111, wherein the first and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO:16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO:18.

### Embodiment 113

The combination of any one of Embodiments 106-112, wherein the first and the second RNAi agents are each independently conjugated to a targeting ligand comprising N-acetyl-galactosamine, wherein the first and the second RNAi agents independently comprise a duplex selected form the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

### Embodiment 114

The combination of any one of Embodiments 103-113, wherein the ratio of the first RNAi agent to the second RNAi agent by weight is in the range of about 1:2 to about 5:1.

### Embodiment 115

The combination of Embodiment 114, wherein the ratio of the first RNAi agent to the second RNAi agent by weight is about 2:1.

### Embodiment 116

The combination of any one of Embodiments 103-115, wherein R^{a}, R^{b} and R^{c} are independently hydrogen, fluoro, bromo, chloro, or CN.

### Embodiment 117

The combination of any one of Embodiments 103-116, wherein R⁴ is C₁-C₃ alkyl.

### Embodiment 118

The combination of Embodiment 117, wherein R⁴ is methyl.

### Embodiment 119

The combination of any one of Embodiment 103-118, wherein R⁶ is C₂-C₆ alkyl optionally substituted with one or more of —OH, fluoro, or C₁-C₄ alkyl optionally substituted with R¹⁰.

### Embodiment 120

The combination of Embodiment 119, wherein R⁶ is C₂-C₆ alkyl substituted with one or more fluoro.

### Embodiment 121

The combination of any one of Embodiments 103-120, wherein each R⁷ is independent hydrogen, halogen or methyl.

### Embodiment 122

The combination of Embodiment 121, wherein at least one R⁷ is hydrogen.

### Embodiment 123

The combination of Embodiment 103 or Embodiment 104, wherein the compound of Formula (I) is selected from the group consisting of:

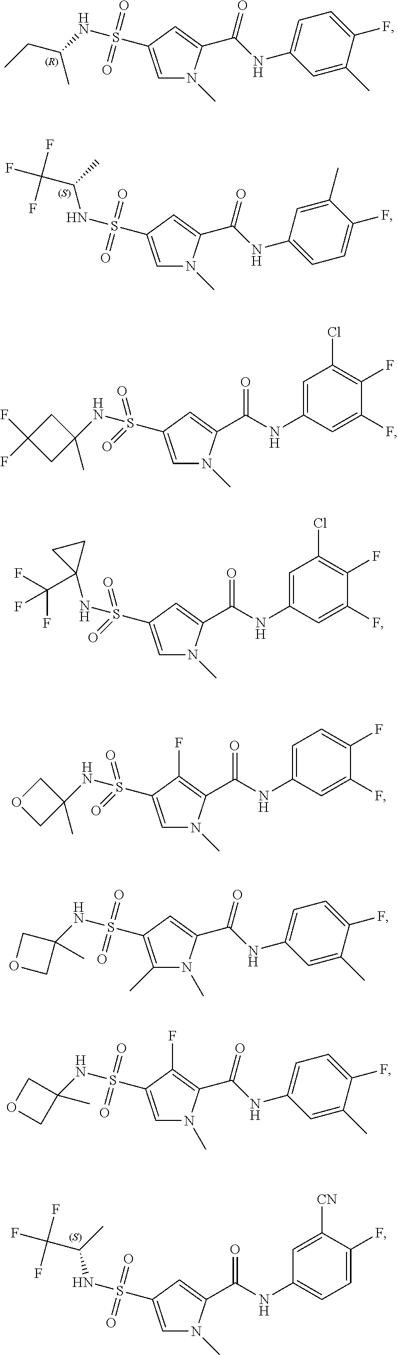

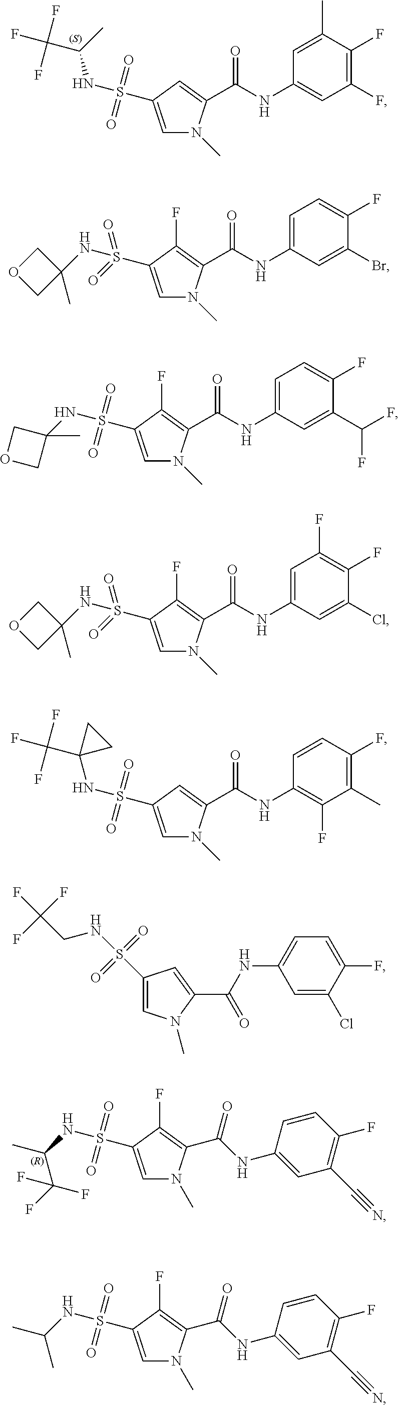

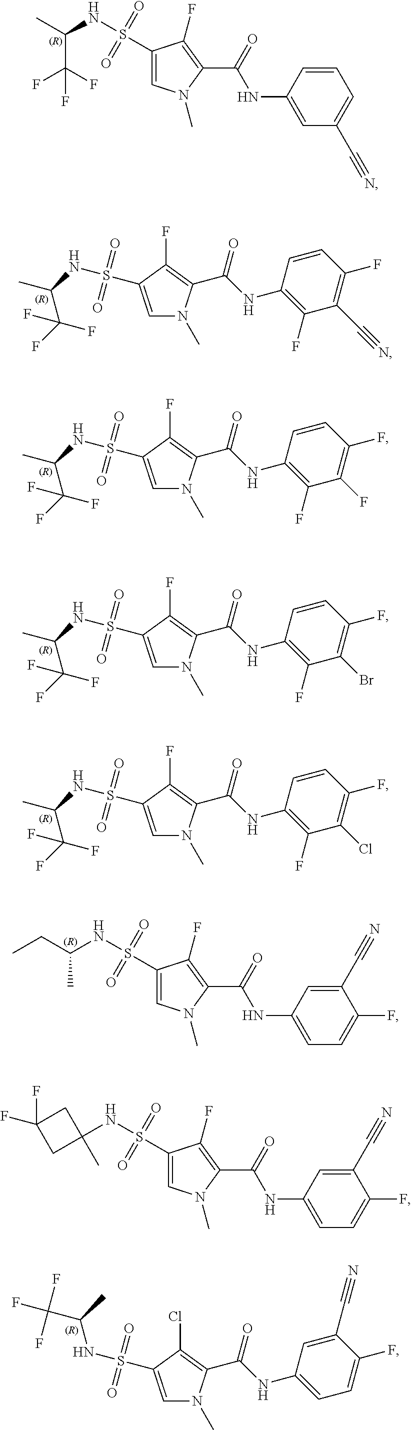

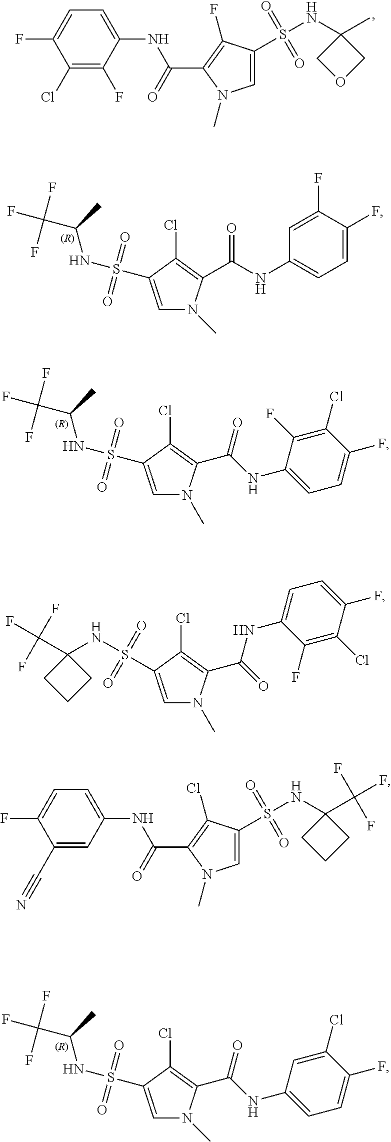

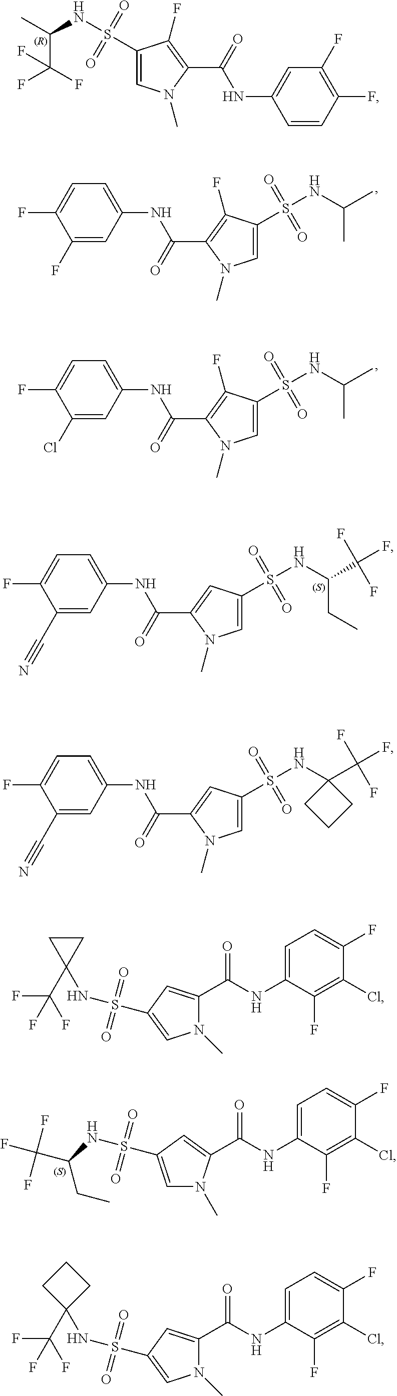

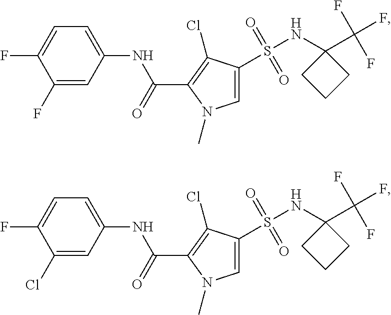 [SMILES: Cn1cc(S(=O)(=O)NC2(C(F)(F)F)CCC2)c(Cl)c1C(=O)Nc1ccc(F)c(F)c1 ; Cn1cc(S(=O)(=O)NC2(C(F)(F)F)CCC2)c(Cl)c1C(=O)Nc1ccc(F)c(Cl)c1]

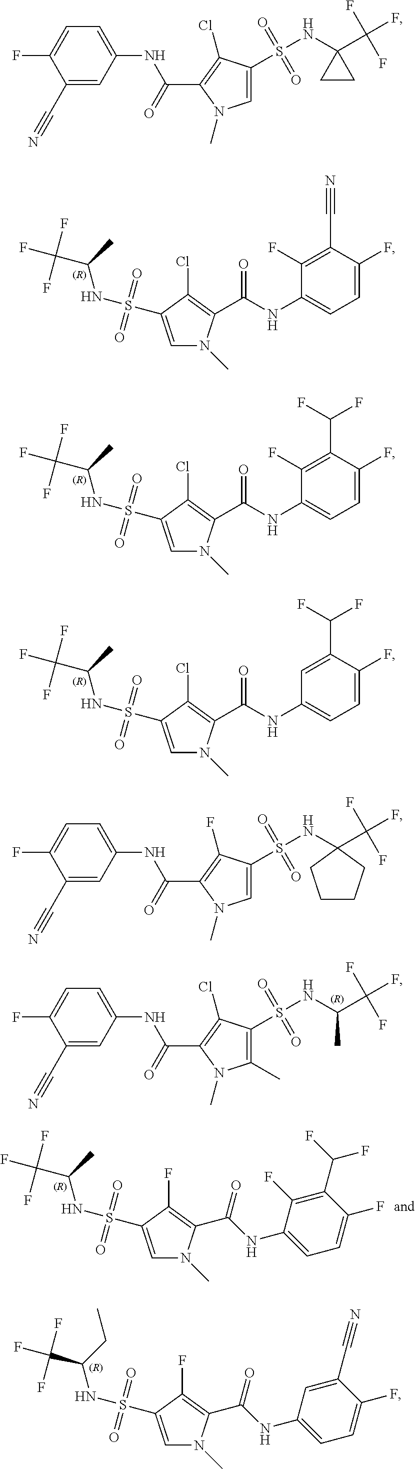

or a pharmaceutically acceptable salt of any of the foregoing.

### Embodiment 124

The combination of any one of Embodiments 103-123, wherein the compound of Formula (I) is selected from the group consisting of

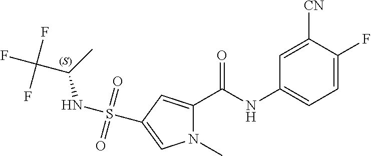

or a pharmaceutically acceptable salt thereof.

### Embodiment 125

The combination of any one of Embodiments 103-123, wherein the compound of Formula (I) is selected from the group consisting of

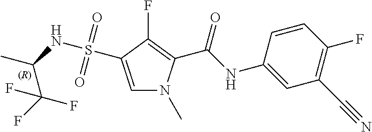

Nor a pharmaceutically acceptable salt thereof.

### Embodiment 126

The combination of Embodiment 103 or Embodiment 104, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

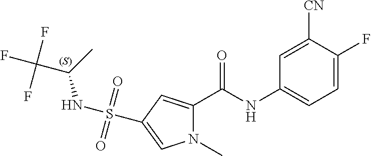

or a pharmaceutically acceptable salt thereof.

### Embodiment 127

The combination of Embodiment 103 or Embodiment 104, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is

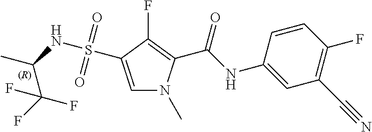

or a pharmaceutically acceptable salt thereof.

### Embodiment 128

A pharmaceutical composition comprising an effective amount of the combination of any one of Embodiments 103-127 and a pharmaceutically acceptable carrier, diluent, excipient, or a combination of any of the foregoing.

### Embodiment 129

An article of manufacture comprising a container enclosing the combination of any one of Embodiments 103-127 or the pharmaceutical composition of Embodiment 128.

### Embodiment 130

A kit comprising the combination of any one of Embodiments 103-127.

### Embodiment 131

The kit of Embodiment 130, wherein the RNAi component is within a first container and the compound of Formula (I) is within a second container.

### Embodiment 132

The kit of Embodiment 130, wherein the RNAi component and the compound of Formula (I) are in the same container.

### EXAMPLES

The following examples are offered to illustrate but not to limit the invention. One of skill in the art will recognize that the following procedures may be modified using methods known to one of ordinary skill in the art.

### Example 1. Clinical Study of the Combination Therapy

A multi-site, Phase I/2a human clinical trial assessing the safety, tolerability, pharmacokinetic, and pharmacodynamic effects of a combination comprising an RNAi component and a capsid assembly modulator in Normal Adult Volunteers was conducted. The study was designed to evaluate, amongst other outcomes, the safety and pharmacological effects of the combination therapy in patients with chronic hepatitis B (CHB). The combination comprised Compound A and an RNAi component comprising of a first RNAi agent comprising SEQ ID NO: 2 and SEQ ID NO: 11 and an effective amount of a second RNAi agent comprising SEQ ID NO: 16 and SEQ ID NO: 8 in a ratio of 2:1. The study subject population included adult males and females, aged 18-55, with or without CHB.

Subjects were given (1) 25-400 mg of the RNAi component as a subcutaneous injection in a single dose or in multiple doses at specific time intervals (depending on the patient cohort) and, in one cohort, subjects were also given (2) 250 mg of compound A as oral tablets (25 mg and 100 mg) daily (from Day 1 to Day 84, inclusive). All patients in all HBV cohorts received NA from day 1 (either TDF or ETV at the approved doses (245 mg for TDF or 0.5 mg for ETV)). Safety assessments included: vital sign assessment (resting heart rate, blood pressure, respiratory rate, temperature); clinical laboratory measurements (biochemistry, hematology, coagulation, urinalysis); resting ECG measurements; assessment of changes in patients' other concurrent medication/therapy; assessment of injection sites; and a 90-day post-EOS pregnancy follow-up call. Efficacy evaluation was made based on a combination of safety/tolerability data (occurrence of adverse events, blood sample analysis); pharmacokinetic data (RNAi component/NUC (nucleos(t)ide inhibitors)/compound A plasma concentrations); and pharmacodynamic data (immunology and virology assessments). The study parameters are summarized in Table 4.

TABLE 4

| | |
|---|---|
| Study title | A Single Dose-Escalating Study to Evaluate the Safety, Tolerability and |
| | Pharmacokinetic Effects of the RNAi component in Normal Adult |
| | Volunteers and Multiple Escalating Doses Evaluating Safety, Tolerability |
| | and Pharmacodynamic Effects in HBV Patients |
| Abbreviations | NHV: Normal Healthy Volunteer |
| | NUC: Nucleos(t)ide inhibitors |
| | CHB: chronic hepatitis B |
| | HBcrAg: HBV core related antigen |
| | HBsAg: HBV S (surface) antigen |
| | HBeAg: HBV E (envelope) antigen |
| Development | Phase I/IIa |
| Phase | |
| Study | Primary Objectives: |
| Objectives | To determine the incidence and frequency of adverse events |
| | possibly or probably related to treatment as a measure of the safety |
| | and tolerability of RNAi component using escalating single doses |
| | in healthy volunteers (NHV) and escalating multiple doses in |
| | patients chronically infected with hepatitis B virus (CHB). |
| | Secondary Objectives: |
| | To evaluate the single-dose pharmacokinetics of RNAi component |
| | in NHVs |
| | To determine the reduction from Day 1 pre-dose baseline to post- |
| | dose nadir of HBsAg in response to RNAi component in patients |
| | chronically infected with HBV (CHB) as a measure of activity. |
| | Exploratory Objectives: |
| | To determine the reduction from Day 1 pre-dose baseline to post- |
| | dose nadir of HBcrAg, HBsAg. HBV RNA (if scientifically |
| | feasible) and HBeAg (e+ patients only), in response RNAi |
| | component (alone or in combination with compound A) in CHB |
| | patients as a measure of activity. |
| | To evaluate the effect of single doses of RNAi component on |
| | cytokines in NHVs. |
| | To evaluate the effect of single escalating doses of RNAi |
| | component on complement factors Bb, CH50, C5a, C4a, and C3a |
| | in NHVs. |
| | To collect plasma samples in NHVs for subsequent metabolite |
| | identification (reported in a separate report outside of this study). |
| | To collect urine samples in NHVs for subsequent determination of |
| | urinary excretion and metabolite identification (reported in a |
| | separate report outside of this study). |
| | To evaluate the effect of multiple doses of RNAi component |
| | (alone or in combination with compound A) on cytokines in CHB |
| | patients. |
| | To evaluate the effect of multiple doses of RNAi component |
| | (alone or in combination with compound A) on HBV patient |
| | immune cell profile including T-cells, NK cells, B cells and |
| | monocytes (if scientifically feasible). |
| | To evaluate the effect of multiple doses of RNAi component |
| | (alone or in combination with compound A) on HBV antigen |
| | specific T-cell response (if scientifically feasible). |
| | To evaluate the effect of interferon response gene single |
| | nucleotide polymorphisms (SNPs) including IL28B on response to |
| | RNAi component (alone or in combination with compound A). |
| | To determine the incidence and frequency of adverse events |
| | possibly or probably related to treatments as a measure of the safety |
| | and the tolerability of RNAi component in combination with |
| | compound A in patients chronically infected with hepatitis B virus |
| | (CHB) (Cohort 12 only). |
| Study Design | Double blind, randomized, placebo-controlled study |
| Sample Size | 30 NHVs; 60-84 CHB patients |
| Study | NHVs: adult males and females; aged 18-55 |
| Population | CHB patients: HBeAg negative or HBeAg positive CHB patients |
| | Cohorts 1-5: NHVs, adult males and females; aged 18-55 |
| | Cohorts 1b, 1c, 2b, 3b, 4b, 5b, 6, 7, 10, 11 and 12: Any CHB patient |
| | regardless of HBeAg or prior therapy status (as long as other Inclusion |
| | and Exclusion criteria are met); |
| | Cohort 8: HBeAg positive treatment naïve patients (no prior chronic |
| | exposure to NUCs or Interferon); |
| | Cohort 9: HBeAg positive NUC experienced patients (on entecavir or |
| | tenofovir for at least 12 months). |
| Study Drugs | RNAi component (dosage of 25-400 mg, depending on Cohort) by |
| | subcataneous injection, either alone or in combination wih compound A |
| | (dosage of 250 mg) by oral tablets; corresponding placebo: normal saline |
| | (0.9%) by subcataneous injection |
| Dosage and | Cohort 1: RNAi component 35 mg on Day 1 |
| Frequency | Cohort 1b: RNAi component 25 mg on Day 1, 29, 57 |
| | Cohort 1c: RNAi component 50 mg on Day 1, 29, 57 |
| | Cohort 2: RNAi component 100 mg on Day 1 |
| | Cohort 2b: RNAi component 100 mg on Day 1, 29, 57 |
| | Cohort 3: RNAi component 200 mg on Day 1 |
| | Cohort 3b: RNAi component 200 mg on Day 1, 29, 57 |
| | Cohort 4: RNAi component 300 mg on Day 1 |
| | Cohort 4b: RNAi component 300 mg on Day 1, 29, 57 |
| | Cohort 5: RNAi component 400 mg on Day 1 |
| | Cohort 5b: RNAi component 400 mg on Day 1, 29, 57 |
| | Cohort 6: RNAi component 100 mg on Day 1, 15, 29 |
| | Cohort 7: RNAi component 100 mg on Day 1, 8, 15 |
| | Cohort 8: RNAi component 300 mg on Day 1, 29, 57 |
| | Cohort 9: RNAi component 300 mg on Day 1, 29, 57 |
| | Cohort 10: RNAi component 200 mg on Day 1, 8, 15 |
| | Cohort 11: RNAi component 300 mg on Day 1, 8, 15 |
| | Cohort 12: RNAi component 200 mg on Day 1, 29, 57 + |
| | compound A 250 mg daily on Day 1 to Day 84 |
| | Placebo: normal saline (0.9%) administered subcutaneously; volume |
| | matched to the corresponding RNAi component dose volume |
| Study Sites | NHVs: Single site in New Zealand |
| | CHB patients: multiple sites in New Zealand, Australia, and Hong Kong |
| Efficacy | Efficacy evaluation is made based on a combination of safety/tolerability |
| Evaluation | data (occurrence of adverse events, blood sample analysis); |
| Criteria | pharmacokinetic data (RNAi component/NUC/compound A plasma |
| | concentrations); and pharmacodynamic data (immunology and virology |
| | assessments). |
| Safety | Safety assessments include: vital signs (resting heart rate, blood pressure, |
| Evaluation | respiratory rate, temperature); clinical laboratory measurements |
| Criteria | (biochemistry, hematology, coagulation, urinalysis); resting ECG |
| | measurements; assessment of changes in patients' other concurrent |
| | medication/therapy; assessment of injection sites; 90-day post-EOS |
| | pregnancy follow-up call. |

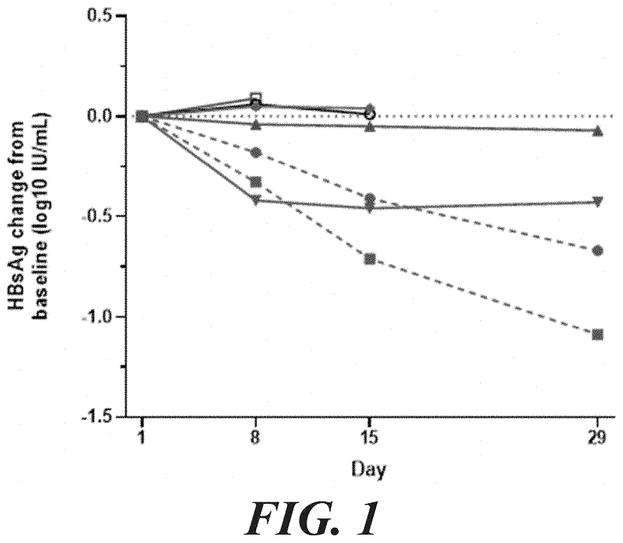

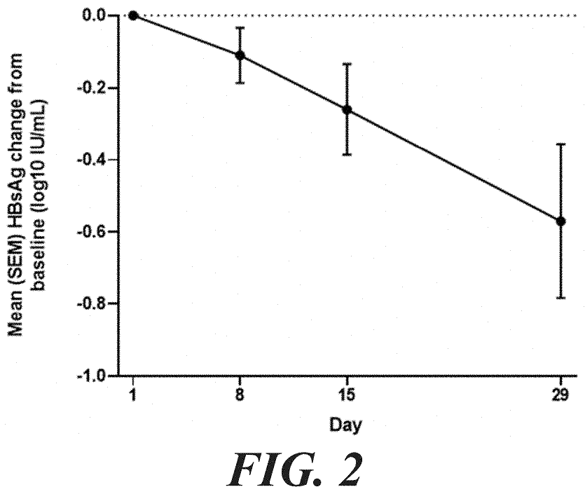

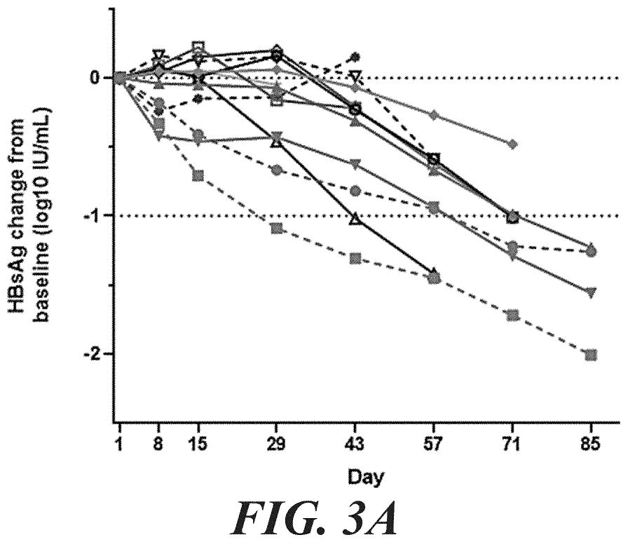

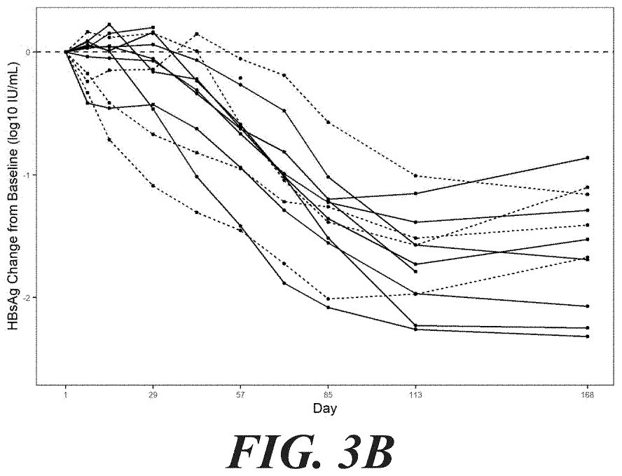

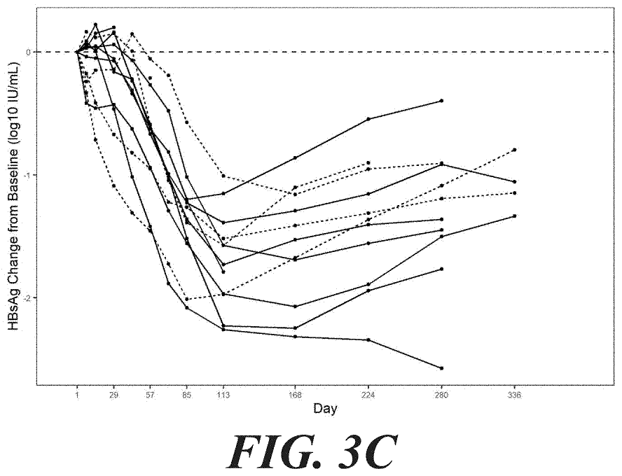

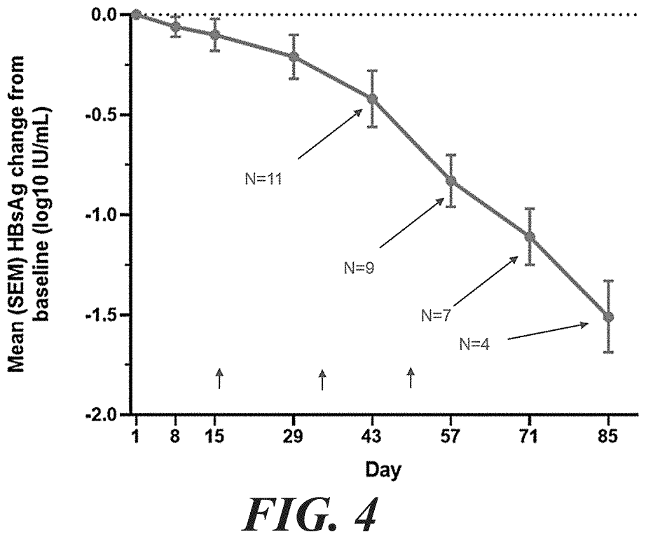

HBsAg changes for individual participants (cohort 12) receiving (1) 200 mg RNAi component, (2) 250 mg compound A daily and (3) NA and the mean HBsAg decline from base line after a single subcutaneous dose of the RNAi component are shown in FIGS. 1 and 2 (up to Day 29). HBsAg changes for individual participants (cohort 12) receiving (1) 200 mg RNAi component, (2) 250 mg compound A daily and (3) NA and the mean HBsAg decline from base line after three subcutaneous doses of the RNAi component on Day 1, 29 and 57 are shown in FIGS. 3A, 3B, 3C (up to Day 85, up to Day 168, and up to Day 336 respectively) and in FIG. 4 (up to Day 85).

Also reported here is a cohort exploring the triple combination therapy of the RNAi component, compound A and an NA (i.e., cohort 12). Efficacy and safety data up to Day 113 (i.e., two months post RNAi component dosing, and one month post compound A dosing). In cohort 12, CHB patients who were HBeAg positive or negative, NA-experienced (regardless of HBV DNA level) or NA-naïve were enrolled and received the following triple combination treatment: (1) three 200 mg RNAi component subcutaneous doses (i.e. on Days 1, 29 and 57); (2) oral compound A 250 mg once daily for 12 weeks (i.e. until Day 85); and (3) all patients either started or continued with NA (ETV or TDF) treatment on Day 1, which continued beyond the end of compound A dosing. Study visits were at screening and on Days 1, 8, 15, 29, 43, 57, 85 and 113, then extended follow up approximately every 2 months for 12 months. Serum viral parameters were assessed, i.e., HBV DNA (lower limit of quantification [LLOQ]: 20 IU/mL), HBV RNA (LLOQ: 1.65 log₁₀ U/mL⁴), HBsAg (LLOQ: 0.05 IU/mL), HBeAg (LLOQ: 0.01 PEIU/mL; value below 0.11 PEIU/mL are reported as not detected), and HBcrAg (LLOQ: 1 kU/mL). Safety assessments included clinical laboratory assessments and adverse events (AEs) assessed from screening through Day 113, as reported here, and through the extended follow-up period.

Baseline characteristics and demographics information for cohort 12 is shown in Table 5. All patients received their planned RNAi component, compound A and NA doses with no treatment discontinuations or dose adjustments.

TABLE 5

| | 12 |
|---|---|
| Cohort | N = 12 |
| Age, years; mean (range) | 46 (34-67) |
| Male, n (%) | 8 (67) |
| Race, n (%) | |
| Asian | 12 (100) |
| BMI, kg/m²; mean (range) | 26.8 (20-36) |
| HBeAg positive/negative, n | 4/8 |
| NA experienced, n (%) | 7 (58) |
| Mean (SEM) HBsAg on Day 1 (IU/mL) | 6612 (116-62, 490) |

BMI: body mass index;
HBeAg: hepatitis B e-antigen;
HBsAg: hepatitis B surface antigen;
NA: nucleosittide analogue;
SEM: standard error of the mean.

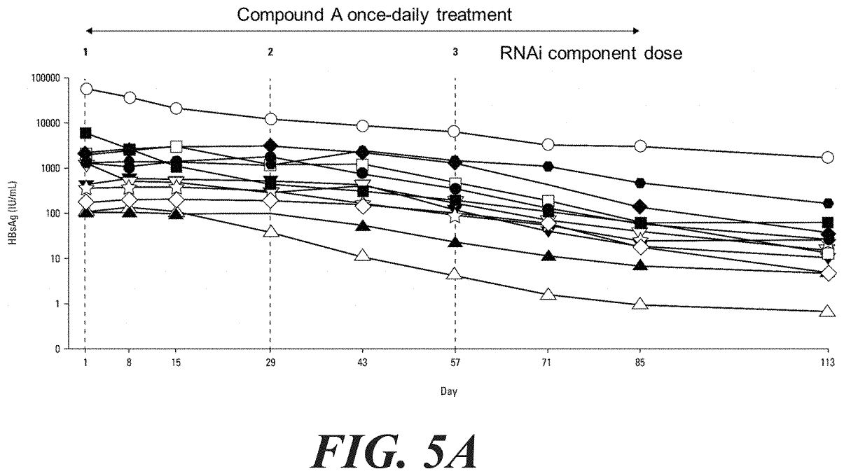

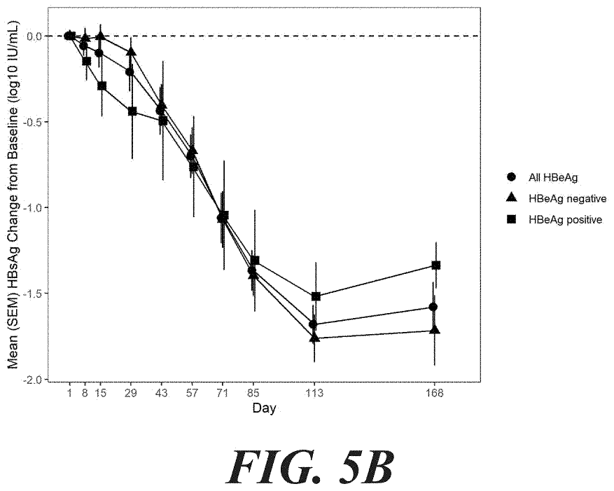

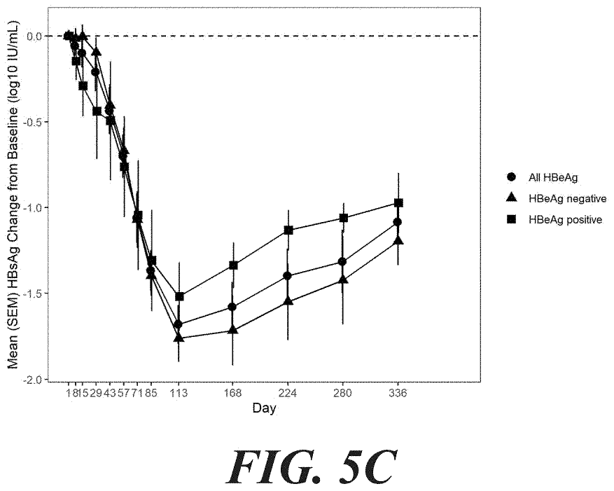

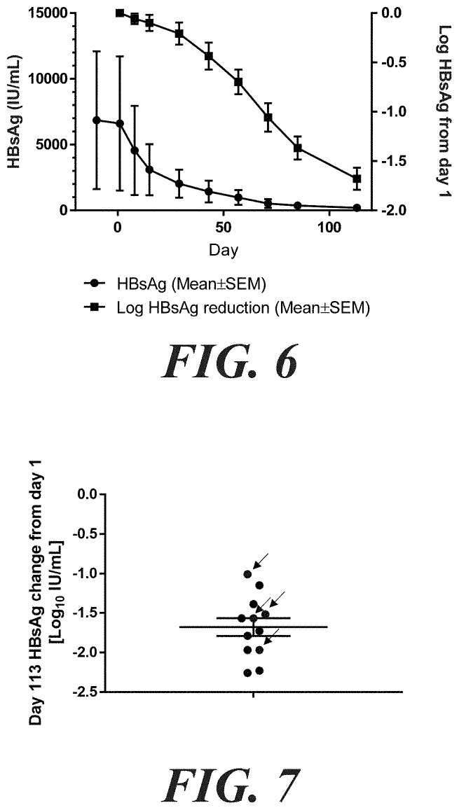

HBsAg changes for individual participants (cohort 12) receiving (1) 200 mg RNAi component, (2) 250 mg compound A daily and (3) NA and the mean HBsAg decline from base line after three subcutaneous doses of the RNAi component on Day 1, 29 and 57 are shown in FIGS. 5A, 5B, 5C (up to Day 113, up to Day 168, and up to Day 336 respectively) and in FIG. 6 (up to Day 113). HBsAg levels declined during treatment in all patients in cohort 12, irrespective of baseline HBsAg level. At Day 113 (typical mean nadir after three RNAi component doses, 56 days after the last dose), mean HBsAg (standard error of the mean [SEM]) log₁₀ IU/mL reduction from Day 1 was 1.7 (0.11), n=12.

HBsAg changes for individual participants (cohort 12) receiving (1) 200 mg RNAi component, (2) 250 mg compound A daily and (3) NA on Day 113 from base line after three subcutaneous doses of the RNAi component on Day 1, 29 and 57 are shown in FIG. 7. All 12 patients in cohort 12 achieved a ≥1.0 log₁₀ IU/mL reduction in HBsAg from Day 1 at the nadir, which was Day 85 for 1 patient and Day 113 for 11 patients. Similar responses were observed for HBeAg positive and negative patients. Individual participants in cohort 12 who are HBeAg negative (N=4, mean nadir: −1.52 log₁₀ IU/mL) and HBeAg positive (N=8, mean nadir: −1.76 log₁₀ IU/mL) showed similar HBsAg reductions on Day 113 from the base line. All patients had HBsAg >100 IU/mL on Day 1 and 10/12 patients achieved HBsAg <100 IU/mL after 12 weeks of therapy with RNAi component, compound A and an NA.

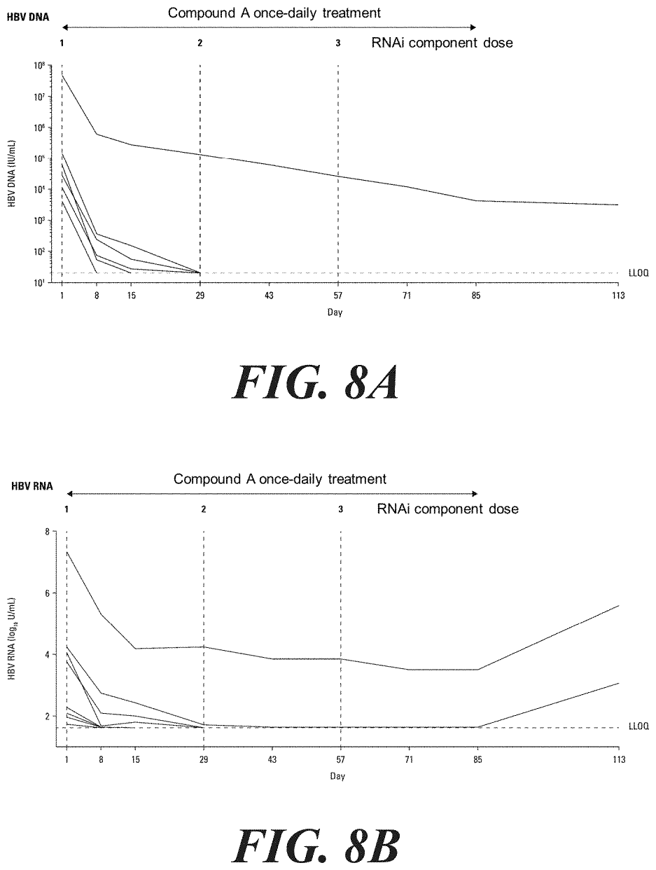

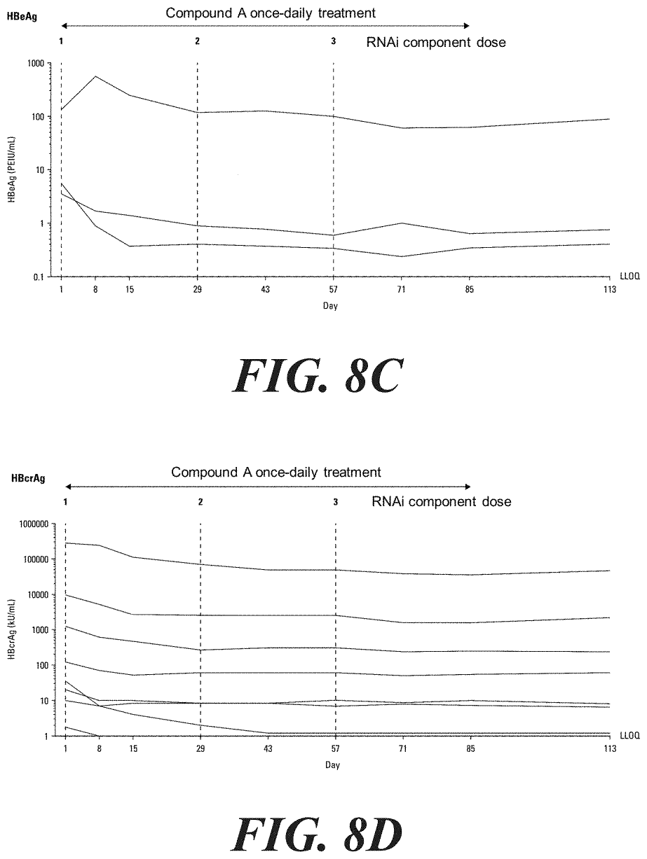

HBV DNA, HBV RNA, HBeAg and HBcrAg changes for individual participants (cohort 12) receiving (1) 200 mg RNAi component, (2) 250 mg compound A daily and (3) NA from base line after three subcutaneous doses of the RNAi component on Day 1, 29 and 57 are shown in FIGS. 8A-8D, respectively. All patients with >1000 IU/mL HBV DNA on day 1 (N=6, 3.7-7.7 log₁₀ IU/mL) had a rapid decline in HBV DNA. One of these six patients had high HBV DNA levels on Day 1 (7.7 log 10 IU/mL), which declined to 3.5 log₁₀ IU/mL on Day 113. One patient had HBV DNA >LLOQ and <1000 IU/mL at baseline (33 IU/mL) and had persistently low HBV DNA levels at or just above the LLOQ (highest value was 54 IU/mL on Day 71)—these data are not included here. And this patient had been on NA treatment for approximately seven years prior to study start. Of the nine patients with quantifiable HBV RNA on Day 1 (1.75-7.5 log 10 U/mL), six had levels <LLOQ by Day 29. Patients positive on Day 1 for HBeAg (N=4) or HBcrAg (N=8) had reductions in there parameters, although these reductions were generally not as pronounced as for the other parameters.

Three doses of 200 mg Q4w RNAi component in combination with daily 250 mg compound A for 12 weeks and an NA daily were generally well tolerated in CHB patients as of the safety data cutoff. No deaths, discontinuations, serious adverse events or sever adverse events were reported. Two AEs of mild respiratory infection, not related to treatment, were reported. There were no clinically significant findings on vital signs, 12-lead electrocardiograms, hematology or clinical chemistry parameters. The only notable treatment-emergent laboratory findings were grade 1, transient isolated alanine aminotransferase elevations (n=5 patients, 57-122 U/L), which resolved with continued dosing and were potentially induced by reduction of viral parameters.

This triple combination was well tolerated, and all CHB patients achieved robust reductions in HBsAg, HBV DNA, and HBV RNA. Reductions in HBeAg and HBcrAg were generally less pronounced during the dosing period. All patients achieved a ≥1.0 log₁₀ IU/mL (90%) reduction (nadir ranged from −1.01 to −2.26 log₁₀ IU/mL) in HBsAg. HBsAg reductions were similar in HBeAg positive and HBeAg negative patients. Studies of longer duration with this triple combination are underway aimed at assessing functional cure rates in patients with CHB.

Also reported here are efficacy and safety data collected in cohorts 1b, 1c and 2b-5b. In cohorts 1b, 1c and 2b-5b, HBeAg positive or negative, NA-experienced or -naïve CHB patients were enrolled and received three subcutaneous RNAi component doses of 25, 50, 100, 200, 300 or 400 mg Q4w on Days 1, 27 and 57. All patients either started (NA-naïve) or continued (NA-experienced) with daily NA (TDF or ETV) treatment on Day 1 and continued beyond the end of RNAi component dosing. Study visits were at screening and on Days 1, 8, 15, 29, 43, 57, 85 and 113, then extended follow up approximately every 2 months for 12 months. Serum viral parameters were assessed, i.e., HBV DNA (lower limit of quantification [LLOQ]: 20 IU/mL), HBV RNA (LLOQ: 1.65 log₁₀ U/mL⁴), HBsAg (LLOQ: 0.05 IU/mL), HBeAg (LLOQ: 0.01 PEIU/mL; value below 0.11 PEIU/mL are reported as not detected), and HBcrAg (LLOQ: 1 kU/mL). Safety assessments included clinical laboratory assessments and adverse events (AEs) assessed from screening through Day 113, as reported here, and through the extended follow-up period.

Baseline characteristics and demographics information for cohorts 1b, 1c, 2b, 3b, 4b and 5b is shown in Table 6. Most patients were NA-experienced ( 40/48, 83%). All patients received their planned RNAi component doses with no treatment discontinuations.

TABLE 6

| Cohort | 1b | 1c | 2b | 3b | 4b | 5b | All patients |
|---|---|---|---|---|---|---|---|
| | 25 mg | 50 mg | 100 mg | 200 mg | 300 mg | 400 mg | |
| | N = 8 | N = 8 | N = 8 | N = 8 | N = 8 | N = 8 | N = 48 |
| | 43 | 48 | 51 | 48 | 52 | 42 | 47 |
| Age, years; mean (range) | (31-52) | (36-58) | (32-66) | (41-57) | (40-63) | (29-61) | (29-66) |
| Male, n (%) | 5 (63) | 6 (75) | 6 (75) | 5 (63) | 8 (100) | 6 (75) | 36 (75) |
| Race, n (%) | | | | | | | |
| Asian | 6 (75) | 5 (63) | 8 (100) | 8 (100) | 5 (63) | 6 (75) | 38 (79) |
| White | 0 | 0 | 0 | 0 | 1 (13) | 0 | 1 (2) |
| Native Hawaiian/other | 1 (13) | 1 (13) | 0 | 0 | 2 (25) | 0 | 4 (8) |
| Pacific Islander | | | | | | | |
| Black/AA | 1 (13) | 0 | 0 | 0 | 0 | 0 | 1 (2) |
| Other | 0 | 2 (25) | 0 | 0 | 0 | 2 (25) | 4 (8) |
| BMI, kg/m²; mean (range) | 27 | 26 | 24 | 25 | 27 | 25 | 26 |
| | (20-44) | (18-40) | (22-29) | (19-32) | (21-36) | (21-36) | (18-44) |
| HBeAg positive/negative, | 2/6 | 3/5 | 1/7 | 1/7 | 3/5 | 1/7 | 11/37 |
| n | | | | | | | |
| NA experienced, n (%) | 4 (50) | 7 (88) | 6 (75) | 8 (100) | 8 (100) | 7 (88) | 40 (83) |
| Mean (SEM) HBsAg on | 6477 | 4595 | 3937 | 3212 | 9381 | 4032 | 5272 |
| Day 1 (IU/mL) | (2876) | (1986) | (2142) | (2453) | (8275) | (1652) | (1558) |

AA: African American;
BMI: body mass index;
HBeAg: hepatitis B e-antigen;
HBsAg: hepatitis B surface antigen;
NA: nucleos(t)ide analogue;
SEM: standard error of the mean.?

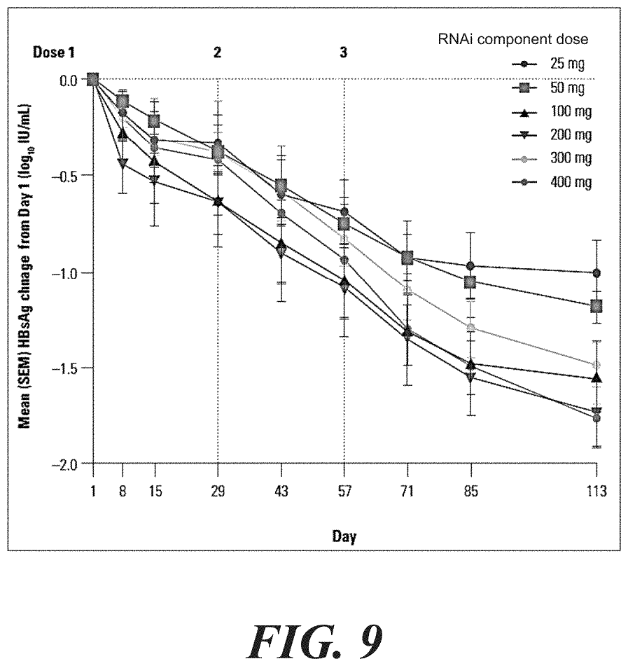

Mean HBsAg changes from Day 1 to Day 113 for CHB patients in cohorts 1b, 1c, 2b, 3b, 4b and 5b are shown in FIG. 9. The patients were given three subcutaneous doses of the RNAi component in the amount of: 25 mg (cohort 1b), 50 mg (cohort 1c), 100 mg (cohort 2b), 200 mg (cohort 3b), 300 mg (cohort 4b) or 400 mg (cohort 5b) on Day 1, 29 and 57. And all patients were given NA daily. RNAi component reduced HBsAg levels at all doses evaluated. At Day 113 (typical mean nadir after three doses, 56 days after last dose), mean HBsAg (SEM) log₁₀ reduction from Day 1 was 1.00 (0.18) with 25 mg, 1.18 (0.08) with 50 mg, 1.54 (0.18) with 100 mg, 1.71 (0.15) with 200 mg, 1.48 (0.11) with 300 mg and 1.75 (0.16) with 400 mg RNAi component.

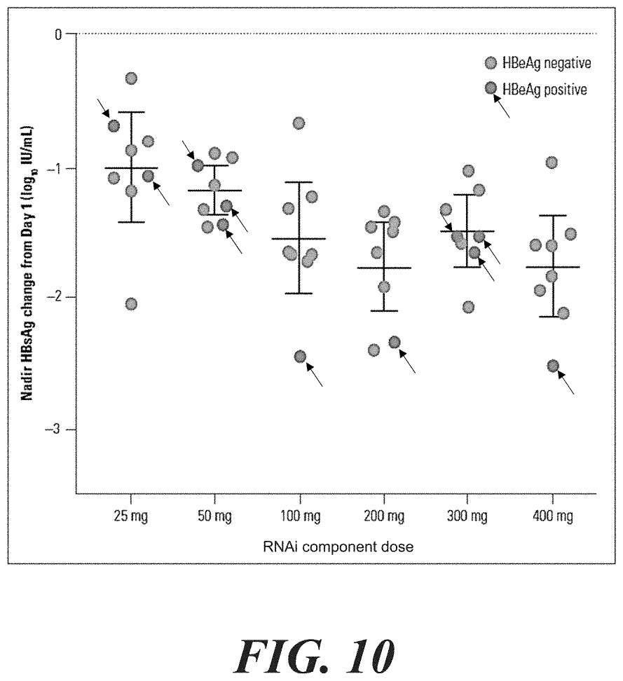

Mean and individual HBsAg changes for on Day 113 from Day 1 for all patients in cohorts 1b, 1c, 2b, 3b, 4b and 5b are shown in FIG. 10. The proportion of patients with Day 113 data who achieved ≥1.0 log₁₀ reduction in HBsAg from Day 1 at nadir was 4/8 (25 mg), 6/8 (50 mg), ⅞ (100 mg), 8/8 (200 mg), 8/8 (300 mg) and 8/8 (400 mg). Similar responses were observed for HBeAg positive and negative patients. HBeAg positive patients (n=11) presented a reduction in HBsAg of −1.52 log₁₀ IU/mL (mean nadir). HBeAg negative patients (n=37) presented a reduction in HBsAg of −1.62 log₁₀ IU/mL (mean nadir). One patient receiving 200 mg had undetectable HBsAg at Day 113, and achieved 1.6 log 10 reduction on Day 15 prior to HBsAg seroclearance. For patients with HBsAg >100 IU/mL (Day 1) and with Day 113 data, 2/7 (25 mg), ⅜ (50 mg), 5/7 (100 mg), ⅚ (200 mg), 6/8 (300 mg) and 5/7 (400 mg) achieved HBsAg <100 IU/mL with RNAi component treatment.

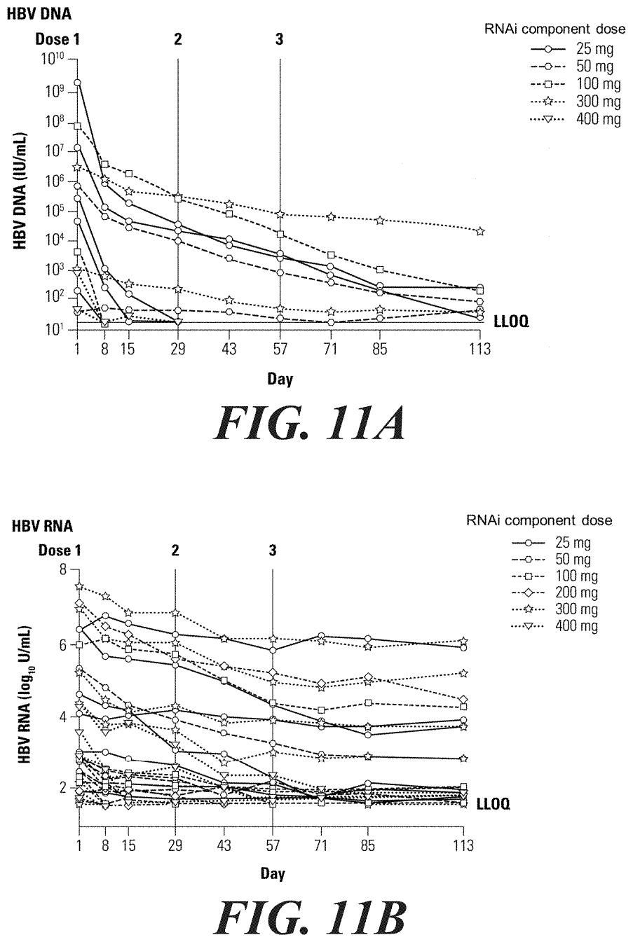

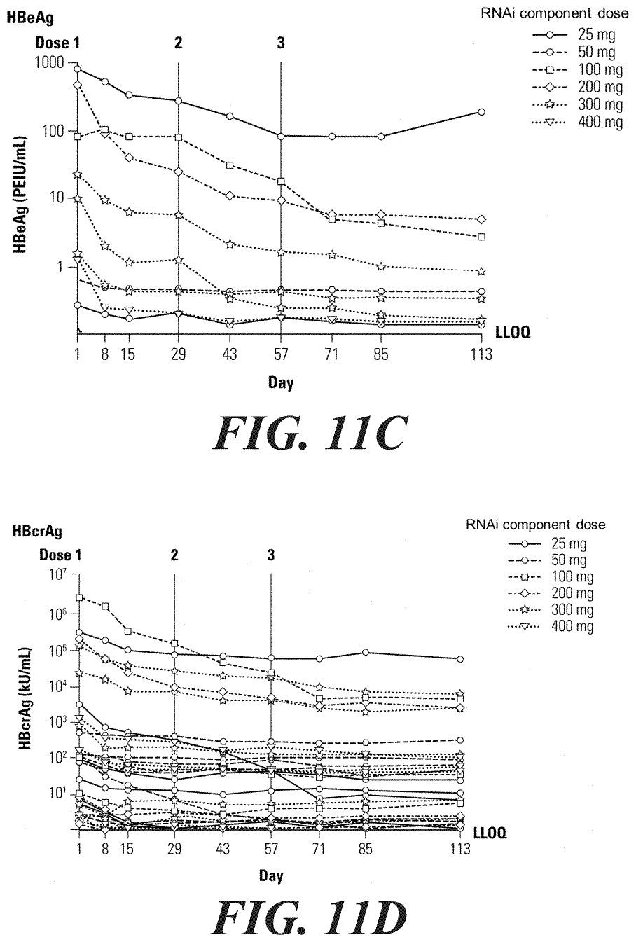

HBV DNA, HBV RNA, HBeAg and HBcrAg changes for individual participants (cohorts 1b, 1c, 2b, 3b, 4b and 5b) receiving 25 mg (cohort 1b), 50 mg (cohort 1c), 100 mg (cohort 2b), 200 mg (cohort 3b), 300 mg (cohort 4b) or 400 mg (cohort 5b) RNAi component on Day 1, 29 and 57 are shown in FIGS. 11A-11D. All patients were given NA daily. For patients with measurable parameters on Day 1, patients had a robust decline in HBV DNA and HBV RNA levels, and reductions in HBeAg and HBcrAg were less pronounced with the RNAi component treatment.

Adverse events (AE) possibly or probably drug related occurring up to and including Day 113 of treatment for participants in cohorts 1b, 1c, 2b, 3b, 4b and 5b are shown in Table 7 below. Safety data for Cohorts 1b to 5b through Day 113 showed that three monthly doses of RNAi component at 25-400 mg with an NA were generally well tolerated in CHB patients. Three non-drug related serious adverse events were reported (anxiety with depression in a single patient and menorrhagia, each requiring hospitalization). The most commonly reported AEs at least possibly drug related consisted of various AEs at the injection site (e.g., discoloration, erythema, bruising, rash), which were all mild and reported in five patients. There were no reports of thrombocytopenia and one report of possibly drug-related Stage 1 acute kidney injury with creatinine increase (from 1.10 mg/dL Day 1 pre-dose to a peak of 1.55 mg/dL on Day 8 and return to 1.06 mg/dL on Day 15), which was treatment emergent but likely due to creatine supplementation and did not lead to treatment interruption or adjustment. A single AE reported of mild, possibly related, abnormal liver function tests (peak alanine aminotransferase [ALT] 136 U/L) was reported, representing the highest treatment-emergent ALT elevation in cohorts 1b to 5b through Day 113 (end of study). There were no cases of simultaneous elevations of ALT >3× upper limit of normal and total bilirubin >2× upper limit of normal.

TABLE 7

| | lb | lc | 2b | 3b | 4b | 5b | All |
|---|---|---|---|---|---|---|---|
| Possibly or probably drug-related | 25 mg | 50 mg | 100 mg | 200 mg | 300 mg | 400 mg | patients |
| AEs in > 2 patients^{a} | N = 8 | N = 8 | N = 8 | N = 8 | N = 8 | N = 8 | N = 48 |
| Injection site discoloration, | 1 mild | 0 | 0 | 0 | 2 mild | 2 mild | 5 |
| injection site erythema, injection | | | | | | | |
| site bruising | | | | | | | |
| Fatigue | 1 mild | 0 | 1 mild | 0 | 0 | 1 mild | 3 |
| Blood creatine phosphokinase | 1 mild | 0 | 0 | 0 | 1 severe | 0 | 2 |
| elevated | | | | | | | |
| Hot flush, flushing | 0 | 1 mild | 0 | 0 | 0 | 1 mild | 2 |
| Blood bilirubin increased, | 0 | 0 | 0 | 1 mild | 1 mild | 0 | 2 |
| hyperbilirubinemia | | | | | | | |
| Pruritus | 1 mild | 0 | 1 mild | 0 | 0 | 0 | 2 |

^{a}MedDRA preferred term aggregated based on similarity?

In CHB patients, RNAi component with an NA had strong activity against HBsAg, HBV DNA and HBV RNA. Reductions in HBeAg and HBcrAg were generally less pronounced. HBsAg reductions were similar in HBeAg positive and HBeAg negative patients. Expanded cohorts with 100-400 mg RNAi component confirmed previous findings that HBsAg declines were similar with these doses; 97% ( 31/32) of these patients achieved a ≥1.0 log₁₀ (90%) reduction in HBsAg. The 25 mg and 50 mg RNAi component doses were active in reducing HBsAg, and appeared less effective than higher doses. HBsAg responses with RNAi component are consistent with its ability to silence HBV RNA from cccDNA and host-integrated viral DNA (which is a major source of HBsAg in certain CHB populations). RNAi component (and NA) treatment was well tolerated at doses up to 400 mg Q4w for three doses. Overall, RNAi component demonstrated anti-HBV characteristics desirable for an RNAi therapy. Studies of longer duration are underway, including triple combinations aimed at functional cure in CHB patients.

### Example 2. Clinical Study of the Combination Therapy

A Phase 2b, multicenter, double-blind, active-controlled, randomized study to investigate the efficacy and safety of different combination regimens, including an RNAi component and/or a capsid assembly modulator, for the treatment of chronic hepatitis B (CHB) virus infection is conducted. The study subject population includes adult males and females, aged 18-65, with CHB. The combination comprises Compound A and an RNAi component comprising an effective amount of a first RNAi agent comprising SEQ ID NO: 2 and SEQ ID NO: 11 and an effective amount of a second RNAi agent comprising SEQ ID NO: 16 and SEQ ID NO: 8 in a ratio of 2:1.

Subjects are given a combination of (1) 40 mg, 100 mg or 200 mg once monthly of the RNAi component by subcutaneous injection, or a corresponding placebo; and (2) 250 mg once daily of compound A as oral tablets (25 mg and 100 mg), or a corresponding placebo; and (3) NA (ETV: nucleoside-naïve patients=0.5 mg daily; lamivudine-refractory patients=1 mg once daily; TDF=300 mg once daily; TAF: 25 mg once daily) as oral tablets (ETV 0.5 mg; TDF 300 mg; TAF 25 mg). The active treatment phase lasts up to 48 weeks in total following a 4-week screening phase. The primary hypothesis of this study is that one or more combination regimens are more efficacious than NA treatment alone, as measured by the primary efficacy endpoint, the proportion of participants with HBsAg seroclearance 24 weeks after completion of all study intervention at Week 48. After 48 weeks, participants who meet the NA treatment completion criteria complete treatment with the RNAi component, compound A and NA, and are monitored closely during a follow-up phase. Participants who do not meet the NA treatment completion criteria continue to receive NA treatment during the 48-week follow up period which could be extended to a maximum duration of 96 weeks. Safety and efficacy evaluation criteria, as well as the study parameters, are summarized in Table 8.

TABLE 8

| | |
|---|---|
| Study title | A Phase 2b, Multicenter, Double-blind, Active-controlled, Randomized |
| | Study to Investigate the Efficacy and Safety of Different Combination |
| | Regimens Including an RNAi component and/or compound A for the |
| | Treatment of Chronic Hepatitis B Virus Infection |
| Abbreviations | NA: Nucleos(t)ide analog |
| | CHB: chronic hepatitis B |
| | HBcrAg: HBV core related antigen |
| | HBsAg: HBV S (surface) antigen |
| | HBeAg: HBV E (envelope) antigen |
| | PK: Pharmacokinetics |
| Development | Phase 2b |
| Phase | |
| Study | Primary Objectives: |
| Objectives | To establish the dose-response relationship for antiviral activity of |
| | 3 doses of RNAi component + NA and to evaluate the efficacy of |
| | combination regimens of RNAi component + NA (with and |
| | without compound A) and of compound A + NA. |
| | Secondary Objectives: |
| | To evaluate the safety and tolerability of the study intervention |
| | throughout the study. |
| | To evaluate the efficacy of the study intervention during follow- |
| | up. |
| | To evaluate efficacy as measured by blood markers (such as |
| | HBsAg, HBeAg*, HBV DNA, and alanine aminotransferase |
| | [ALT]) during study intervention and follow-up. |
| | To evaluate the frequency of virologic breakthrough. |
| | To evaluate the efficacy of NA re-treatment in participants who |
| | meet the criteria for NA re-treatment. |
| | To evaluate the PK of RNAi component, compound A, and NA, as |
| | applicable. |
| | Exploratory Objectives: |
| | To explore changes in the severity of liver disease at the end of |
| | follow-up versus baseline. |
| | To explore the relationship between PK parameters (RNAi |
| | component and/or compound A and/or NA) and selected PD |
| | parameters of efficacy and/or safety, as applicable. |
| | To explore efficacy as measured by HBV RNA and hepatitis B |
| | core-related antigen (HBcrAg) during study intervention and |
| | follow-up. |
| | To explore the impact of the baseline HBeAg and treatment status |
| | factors on efficacy as measured by primary and secondary |
| | endpoints. |
| | To explore the association between viral and host baseline factors |
| | with efficacy and safety. |
| | To explore changes in the HBV genome sequence during study |
| | intervention and follow-up. |
| | To explore the effect of any baseline variation in the HBV genome |
| | on efficacy. |
| | To explore HBV-specific T-cell response during study |
| | intervention and follow-up. |
| | To explore the impact of study intervention on how participants |
| | feel and function using patient-reported outcomes (PROs) during |
| | study intervention and follow-up. |
| | * in HBeAg positive participants only |
| Study Design | Randomized, double-blind, double-dummy, active-controlled, dose- |
| | finding, parallel, multicenter, interventional study |
| Sample Size | Target sample size of 450 participants |
| Study | Adult males and females, aged 18-65, with CHB |
| Population | Arm 1: 90 participants |
| | Arm 2: 90 participants |
| | Arm 3: 90 participants |
| | Arm 4: 90 participants |
| | Arm 5: 45 participants |
| | Arm 6: 45 participants |
| Study Drugs | RNAi component (dose of 40 mg, 100 mg or 200 mg) by subcutaneous |
| | injection; compound A (dose of 250 mg) by oral tablet; Entecavir (ETV) |
| | (dose of 0.5-1 mg) by oral tablet; Tenofovir disoproxil fumarate (TDF) |
| | (dose of 300 mg) by oral tablet; Tenofovir alafenamide (TAF) (dose of 25 |
| | mg) by oral tablet; Tenofovir disoproxil fumarate (TDF) (dose of 300 mg) |
| | by oral tablet; corresponding placebos: 0.9% saline solution by |
| | subcutaneous injection, oral tablets |
| Dosage and | Arm 1: RNAi component (100 mg) + compound A (250 mg) + NA (ETV, |
| Frequency | TDF, or TAF) |
| | Arm 2: RNAi component (200 mg) + placebo + NA (ETV, TDF, or TAF) |
| | Arm 3: RNAi component (100 mg) + placebo + NA (ETV, TDF, or TAF) |
| | Arm 4: RNAi component (40 mg) + placebo + NA (ETV, TDF, or TAF) |
| | Arm 5: Placebo + compound A (250 mg) + NA (ETV, TDF, or TAF) |
| | Arm 6 (control): Placebo + placebo + NA (ETV, TDF, or TAF) |
| | RNAi component = 40 mg, 100 mg or 200 mg once monthly |
| | compound A = 250 mg once daily |
| | ETV: nucleoside-naïve patients = 0.5 mg daily; lamivudine-refractory |
| | patients = 1 mg once daily (but preferably treated with TDF or TAF |
| | instead) |
| | TDF = 300 mg once daily |
| | TAF = 25 mg once daily |
| | Placebos: normal saline (0.9%) by subcutaneous injection (for RNAi |
| | component); oral tablets (for compound A); frequency to match |
| | corresponding drug dosage. |
| | All treatments to last up to a total of 48 weeks; addition of screening and |
| | follow-up phases means the duration of individual participation will be |
| | 100-150 weeks. |
| | NA Treatment Completion Criteria: |
| | The participant has ALT < 3x ULN, AND |
| | The participant has HBV DNA < LLOQ, AND |
| | The participant is HBeAg-negative, AND |
| | The participant has HBsAg < 10 IU/mL. |
| Study Sites | Multicenter |
| Primary | Proportion of participants with HBsAg seroclearance 24 weeks |
| Efficacy | after completion of all study intervention at Week 48. |
| Endpoint | |
| Secondary | Proportion of participants with (serious) adverse events ([S]AEs) |
| Efficacy | and abnormalities in clinical laboratory tests (including |
| Endpoint | hematology, blood biochemistry, blood coagulation, and |
| | urinalysis), 12-lead electrocardiograms (ECGs), and vital signs. |
| | Proportion of participants with HBsAg seroclearance 24 weeks |
| | after completion of all study intervention at Week 48. |
| | Proportion of participants with HBsAg seroclearance 48 weeks |
| | after completion of all study intervention at Week 48. |
| | Proportion of participants with HBV DNA < lower limit of |
| | quantification (LLOQ) 24 and 48 weeks after completion of all |
| | study intervention at Week 48. |
| | Proportion of participants meeting the NA treatment completion |
| | criteria during follow-up. |
| | Proportion of participants with HBsAg seroclearance 24 and 48 |
| | weeks after completion of NA treatment at any time during |
| | follow-up. |
| | Frequency of relapse. |
| | Proportion of participants meeting the NA treatment completion |
| | criteria during follow-up. |
| | Proportion of participants not requiring NA re-treatment during |
| | follow-up. |
| | Proportion of participants who reach HBV DNA undetectability |
| | after re-start of NA treatment during follow-up. |
| | Proportion of participants with (sustained) reduction, suppression, |
| | and/or seroclearance considering single and multiple markers |
| | (such as HBsAg, HBeAg*, HBV DNA and ALT). |
| | Proportion of participants with HBsAg and HBeAg* |
| | seroconversion. |
| | Change from baseline over time in HBsAg, HBeAg*, and HBV |
| | DNA. |
| | Time to achieve HBsAg and HBeAg* seroclearance. |
| | Proportion of participants with HBsAg levels and/or changes from |
| | baseline below/above different cut-offs (eg, HBsAg < 100 IU/mL |
| | or > 1 log10 IU/mL reduction in HBsAg from baseline). |
| | Proportion of HBeAg-positive participants with HBeAg* levels |
| | and/or changes from baseline below/above different cut-offs. |
| | Proportion of participants with HBV DNA levels and/or changes |
| | from baseline below/above different cut-offs (eg, < lower limit of |
| | quantification [LLOQ] of the assay). |
| | Proportion of participants with ALT decrease and normalization. |
| | Proportion of participants with virologic breakthrough. |
| | Proportion of participants meeting the NA treatment completion |
| | criteria. |
| | PK parameters of RNAi component, compound A, and NA, as |
| | applicable. |
| | * in HBeAg positive participants only |
| Exploratory | Changes in fibrosis (according to Fibroscan liver stiffness |
| Efficacy | measurements). |
| Endpoint | Relationship between various PK parameters (RNAi component |
| | and/or compound A and/or NA) and selected efficacy and/or |
| | safety endpoints, as applicable. |
| | Changes from baseline in HBV RNA and HBcrAg levels. |
| | Time to reach undetectability of HBV RNA and HBcrAg. |
| | Correlation of viral and host baseline characteristics (such as age, |
| | gender, body mass index [BMI]) with selected efficacy and safety |
| | variables. |
| | Emergence of intervention-associated mutations. |
| | Correlation of HBV genome sequence with selected efficacy |
| | parameters. |
| | Changes from baseline in HBV-specific T-cell responses. |
| | Changes over time in score on the Hepatitis B Quality of Life |
| | (HBQOL) scale and subscales |
| | Changes over time in total score on the HBV-specific self-stigma |
| | PRO scale. |
| | Shifts in score on the Patient Global Impression of Change (PGIC) |
| | scale. |
| Efficacy | Qualitative and quantitative HBsAg and HBeAg, and quantitative |
| Evaluation | HBcrAg as well as anti-HBs and anti-HBe antibodies are determined |
| Criteria | using standard serologic assays in a central laboratory. |
| | HBV DNA and HBV RNA are quantified at a central laboratory using in |
| | vitro nucleic acid amplification tests for the quantification of HBV DNA |
| | and HBV RNA. |
| | Assessments are performed to determine changes in fibrosis. |
| | Samples may be used for additional exploratory assessments analyzing the |
| | serologic and virologic characteristics of HBV infection and efficacy or |
| | safety of the study intervention. |
| Safety | Safety and tolerability are assessed throughout the study from the time |
| Evaluation | that the informed consent form (ICF) is signed until the completion of the |
| Criteria | last study-related activity, which may include contact for follow-up of |
| | safety. The evaluations of safety and tolerability include monitoring of |
| | (serious) adverse effects, physical examinations, vital signs |
| | measurements, triplicate 12-lead ECGs, and clinical laboratory tests |
| | (including hematology, blood biochemistry, blood coagulation, and |
| | urinalysis). Any clinically relevant changes occurring during the study |
| | must be recorded in the AE section of the case report form (CRF). |
| | Specific toxicity management plans in line with the known |
| | pharmacological profile of the study intervention (and the drug classes) |
| | evaluated in this study are implemented. |

1: A method of treating a viral infection in a subject, comprising administering to the subject an effective amount of a combination comprising an RNAi component and a compound of Formula (I), wherein:
(a) the RNAi component comprises
(i) a first RNAi agent comprising: an antisense strand comprising a nucleotide sequence of any one of the following: SEQ ID NO:1, SEQ ID NO:2, SEQ ID NO:3, SEQ ID NO:4, SEQ ID NO:5, SEQ ID NO:6, and SEQ ID NO:7, and a sense strand comprising a nucleotide sequence of any one of the following: SEQ ID NO:10, SEQ ID NO:11, SEQ ID NO:12, SEQ ID NO:13, SEQ ID NO:14, and SEQ ID NO:15; and
(ii) a second RNAi agent comprising: an antisense strand comprising a nucleotide sequence of any one of the following: SEQ ID NO:8 and SEQ ID NO:9, and a sense strand comprising a nucleotide sequence of any one of the following: SEQ ID NO:16, SEQ ID NO:17, SEQ ID NO:18, and SEQ ID NO:19; and
(b) the compound of Formula (I) is 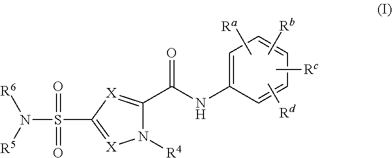
or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R^{d} is hydrogen or fluoro;
R⁴ is hydrogen, C₁-C₃ alkyl or C₃-C₄ cycloalkyl;
R⁵ is hydrogen;
R⁶ is selected from the group consisting of C₂-C₆ alkyl, C₁-C₄ alkyl-R⁸ optionally substituted with one or more fluoro, C₁-C₄ alkyl-R⁹ optionally substituted with one or more fluoro, and a 3-7 membered mono or polycyclic saturated ring optionally containing one or more heteroatoms each independently selected from the group consisting of O, S and N, such 3-7 membered saturated ring or C₂-C₆ alkyl optionally substituted with one or more substituents each independently selected from the group consisting of hydrogen, —OH, fluoro, oxo, R⁹, R¹⁰ and C₁-C₄ alkyl optionally substituted with R¹⁰;
R⁷ is hydrogen, —CN, halogen, —CHF₂, —CF₂-methyl, —CH₂F, —CF₃, C₁-C₃ alkyl optionally substituted with methoxy, C₂-C₃ alkenyl or C₃-C₄ cycloalkyl;
R⁸ is 3-7 membered saturated ring optionally containing one or more heteroatoms each independently selected from the group consisting of O, S and N, such 3-7 membered saturated ring optionally substituted with one or more C₁-C₄ alkyl optionally substituted with R¹⁰;
R⁹ is C₁-C₄ alkyloxy, —SO₂-methyl, —C(═O)—OR¹¹ or —C(═O)—N(R¹¹)₂;
R¹⁰ is —CN, —OH, fluoro, —CHF₂, —CH₂F or —CF₃; and
R¹¹ is hydrogen or C₁-C₃ alkyl.

2: The method of claim 1, wherein the first or the second RNAi agent comprises at least one modified nucleotide or at least one modified internucleoside linkage.

3: The method of claim 1, wherein substantially all of the nucleotides in the first and the second RNAi agents are modified nucleotides.

4: The method of claim 1, wherein the first or the second RNAi agent further comprises a targeting ligand that is conjugated to the first or the second RNAi agent.

5: The method of claim 4, wherein the targeting ligand comprises N-acetyl-galactosamine.

6: The method claim 5, wherein the targeting ligand is selected from the group consisting of (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s.

7: The method of claim 6, wherein the targeting ligand is (NAG25), (NAG25)s, (NAG31), (NAG31)s, (NAG37), or (NAG37)s.

8: The method of claim 4, wherein the targeting ligand is conjugated to the sense strand of the first or the second RNAi agent.

9: The method of claim 8, wherein the targeting ligand is conjugated to the 5′ terminus of the sense stand of the first or the second RNAi agent.

10: The method of claim 1, wherein the first and the second RNAi agents independently comprise a duplex selected from the group consisting of:
an antisense strand comprising SEQ ID NO: 1 and a sense strand comprising SEQ ID NO: 10;
an antisense strand comprising SEQ ID NO: 2 and a sense strand comprising SEQ ID NO: 11;
an antisense strand comprising SEQ ID NO: 3 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO:16;
an antisense strand comprising SEQ ID NO: 8 and a sense strand comprising SEQ ID NO: 17;
an antisense strand comprising SEQ ID NO: 2 and a sense strand comprising SEQ ID NO: 12; and
an antisense strand comprising SEQ ID NO: 8 and a sense strand comprising SEQ ID NO:18.

11: The method of claim 1, wherein the first and the second RNAi agents are each independently conjugated to a targeting ligand comprising N-acetyl-galactosamine, and the first and the second RNAi agents independently comprise a duplex selected from the group consisting of:
an antisense strand comprising SEQ ID NO: 2 and a sense strand comprising SEQ ID NO: 11;
an antisense strand comprising SEQ ID NO: 4 and a sense strand comprising SEQ ID NO: 12;
an antisense strand comprising SEQ ID NO: 8 and a sense strand comprising SEQ ID NO: 16;
an antisense strand comprising SEQ ID NO: 2 and a sense strand comprising SEQ ID NO: 13; and
an antisense strand comprising SEQ ID NO: 8 and a sense strand comprising SEQ ID NO: 18.

12: The method of claim 1, wherein the ratio of the first RNAi agent to the second RNAi agent by weight is in the range of about 1:2 to about 5:1.

13: The method of claim 12, wherein the ratio of the first RNAi agent to the second RNAi agent by weight is about 2:1.

14: The method of claim 1, wherein the compound of Formula (I) is a compound of Formula (II): 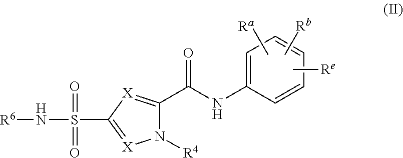
or a pharmaceutically acceptable salt thereof, wherein:
each X is independently CR⁷;
R^{a}, R^{b} and R^{c} are independently selected from the group consisting of hydrogen, halogen, —CHF₂, —CF₂-methyl, —CH₂F, —CF₃, —OCF₃, —CN, C₁-C₃ alkyl and C₃-C₄ cycloalkyl;
R⁴ is hydrogen or C₁-C₃ alkyl;
R⁶ is selected from the group consisting of C₂-C₆ alkyl and a 3-7 membered mono or polycyclic saturated ring optionally containing one or more heteroatoms each independently selected from the group consisting of O, S and N, wherein the C₂-C₆ alkyl or the 3-7 membered saturated ring is optionally substituted with one or more substituents each independently selected from the group consisting of hydrogen, —OH, fluoro and C₁-C₄ alkyl optionally substituted with R¹⁰,
R⁷ is hydrogen, halogen or C₁-C₃ alkyl; and
R¹⁰ is —CN, —OH, fluoro, —CHF₂, —CH₂F or —CF₃.

15: The method of claim 14, wherein R^{a}, R^{b} and R^{c} are independently hydrogen, fluoro, bromo, chloro, or CN.

16: The method of claim 14, wherein R⁴ is C₁-C₃ alkyl.

17: The method claim 16, wherein R⁴ is methyl.

18: The method of claim 14, wherein R⁶ is C₂-C₆ alkyl optionally substituted with one or more of —OH, fluoro, or C₁-C₄ alkyl optionally substituted with R⁰.

19: The method of claim 18, wherein R⁶ is C₂-C₆ alkyl substituted with one or more fluoro.

20: The method of claim 14, wherein each R⁷ is independent hydrogen, halogen or methyl.

21: The method of claim 20, wherein at least one R⁷ is hydrogen.

22: The method of claim 1, wherein the compound of Formula (I) is selected from the group consisting of: 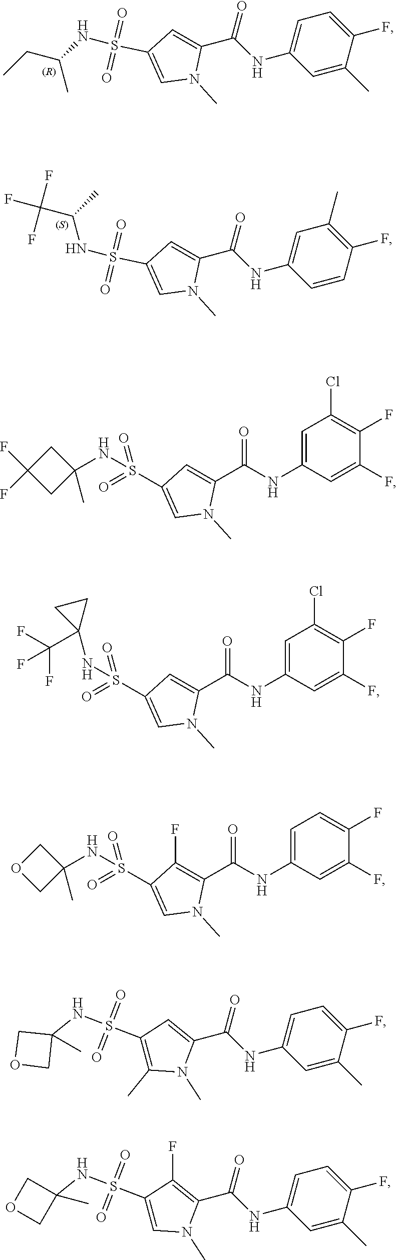 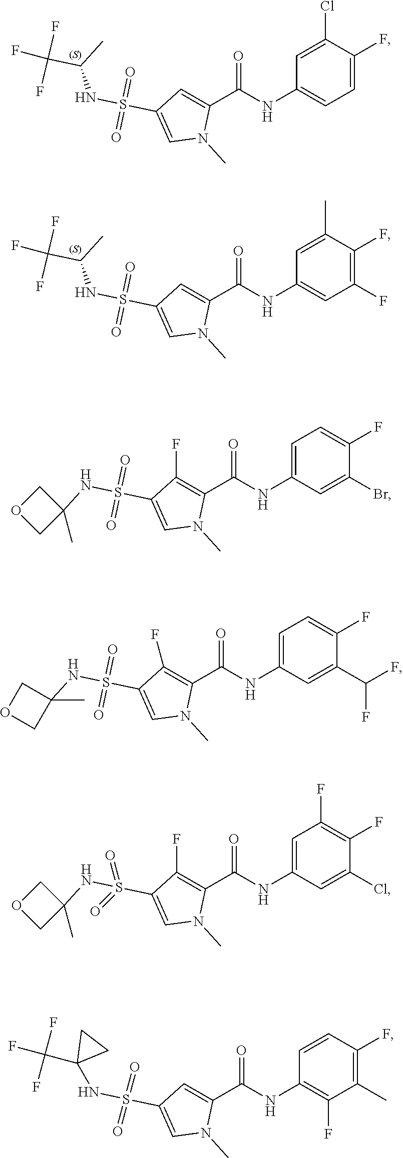 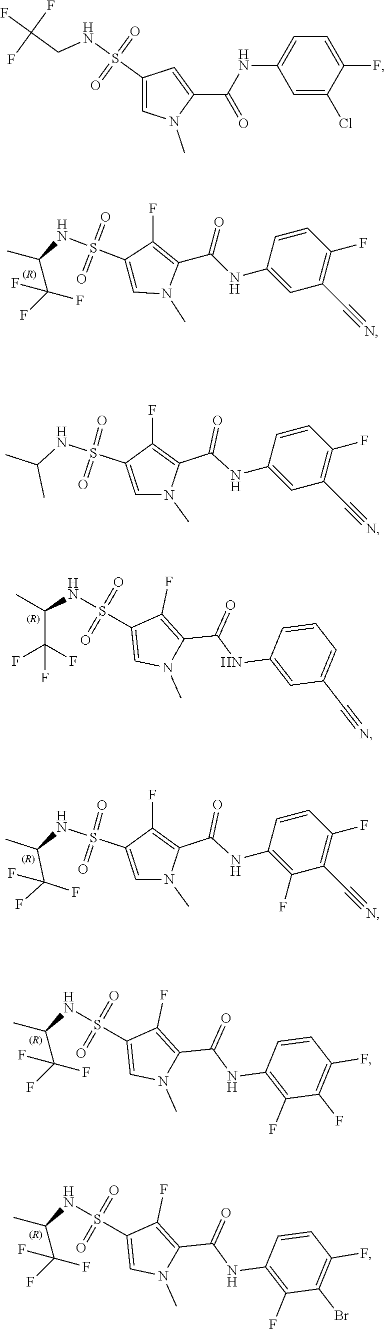 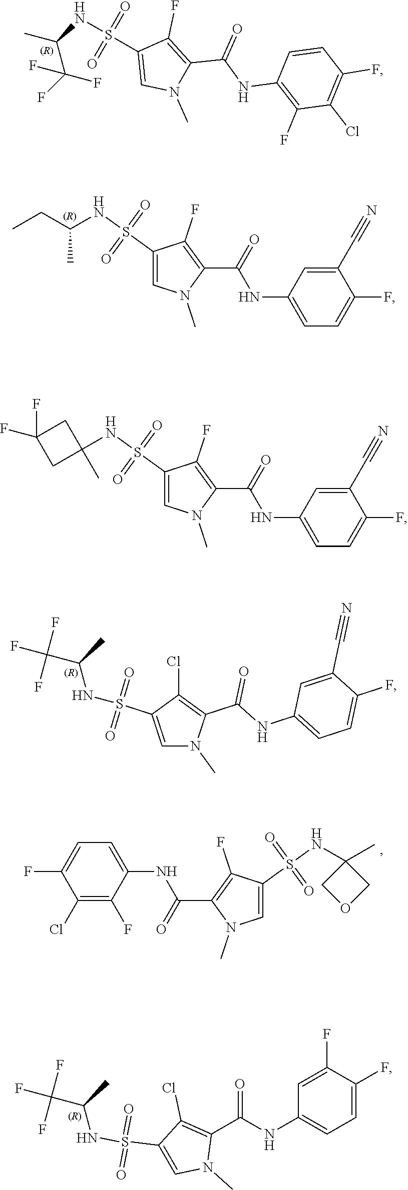 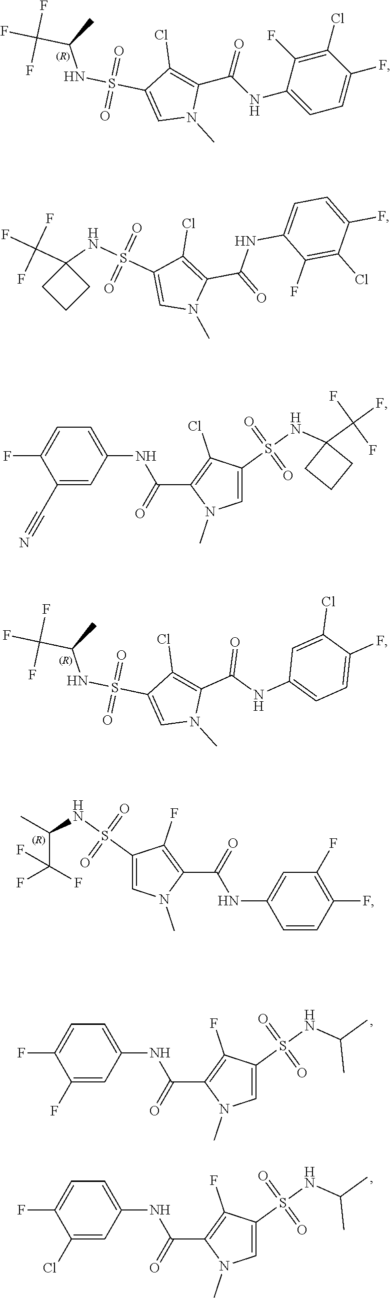 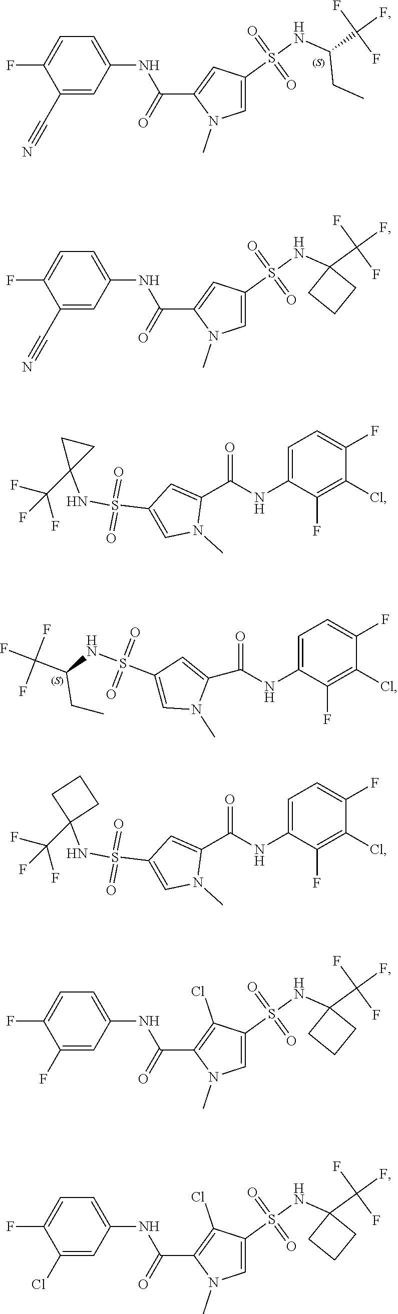 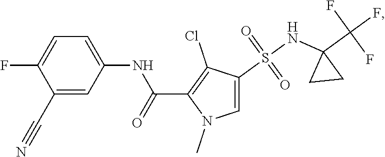 [SMILES: Cn1cc(S(=O)(=O)NC2(C(F)(F)F)CC2)c(Cl)c1C(=O)Nc1ccc(F)c(C#N)c1] 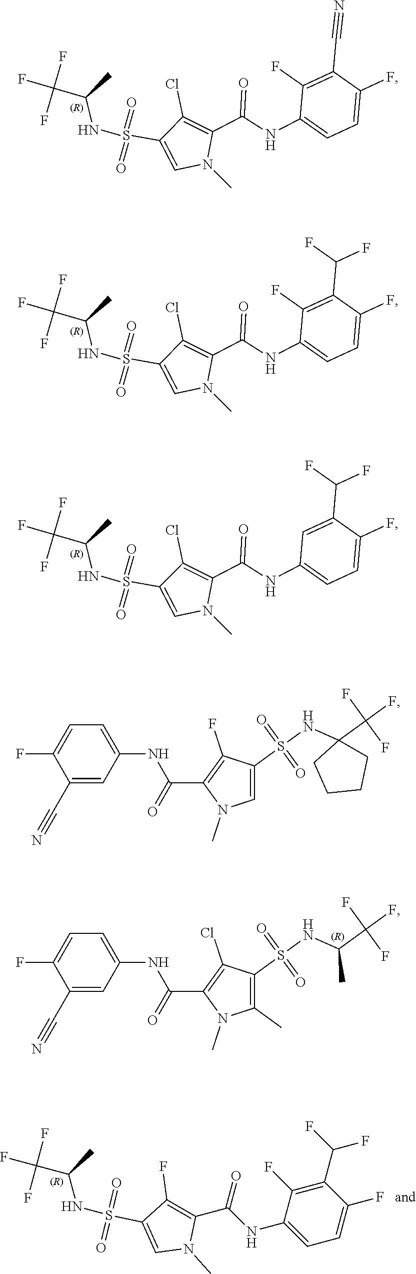 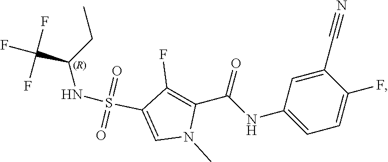
or a pharmaceutically acceptable salt of any of the foregoing.

23: The method of claim 1, wherein the compound of Formula (I) is 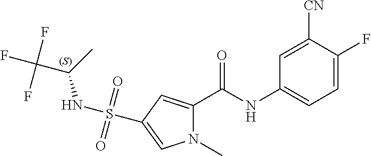
or a pharmaceutically acceptable salt thereof.

24: The method of claim 1, wherein the compound of Formula (I) is 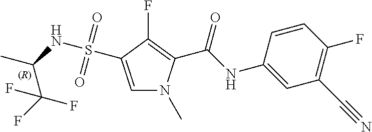
or a pharmaceutically acceptable salt thereof.

25: The method of claim 1, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is 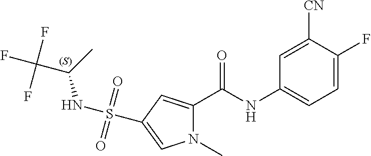
a pharmaceutically acceptable salt thereof.

26: The method of claim 1, wherein the first and the second RNAi agents are each independently conjugated to (NAG37)s, the first RNAi agent comprises an antisense strand comprising SEQ ID NO: 2 and a sense strand comprising SEQ ID NO: 11, the second RNAi agent comprises an antisense strand comprising SEQ ID NO: 8 and a sense strand comprising SEQ ID NO: 16 and the compound of Formula (I) is 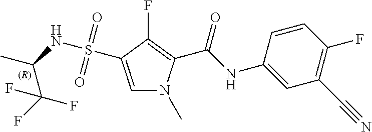
or a pharmaceutically acceptable salt thereof.

27: The method of claim 1, wherein the RNAi component is administered to the subject once monthly in a dose of about 40-200 mg.

28: The method of claim 1, wherein the RNAi component is administered to the subject once monthly in a dose of about 50-200 mg.

29: The method of claim 1, wherein the compound of Formula (I) or a pharmaceutically acceptable salt thereof is administered to the subject in a daily dose of about 100-500 mg.

30: The method of claim 1, wherein the RNAi component is administered to the subject via intravenous or subcutaneous injection.

31: The method of claim 1, wherein the compound of Formula (I) or a pharmaceutically acceptable salt thereof is administered to the subject orally.

32: The method of claim 1, wherein the RNAi component is administered simultaneously or sequentially with the compound of Formula (I) or a pharmaceutically acceptable salt thereof.

33: The method of claim 1, wherein the RNAi component is administered separately from the compound of Formula (I) or a pharmaceutically acceptable salt thereof.

34: The method of claim 1, wherein the subject has been receiving the compound of Formula (I) or a pharmaceutically acceptable salt thereof for at least about 1 month.

35: The method of claim 1, wherein the combination further comprises a nucleoside analog.

36: The method of claim 35, wherein the nucleoside analog is entecavir, tenofovir disoproxil fumarate or tenofovir alafenamide.

37: The method of claim 36, wherein the nucleoside analog is entecavir administered to the subject in a daily dose of about 0.1-5 mg.

38: The method of claim 36, wherein the nucleoside analog is tenofovir administered to the subject in a daily dose of about 5-50 mg of tenofovir alafenamide or about 200-500 mg of tenofovir disoproxil fumarate.

39: The method of claim 1, wherein the viral infection is caused by Hepatitis B Virus.

40: The method of claim 1, wherein the viral infection is a chronic Hepatitis B infection.

41: The method of claim 1, wherein the subject is co-infected with Hepatitis B Virus and another virus.
